(19) United States
(12) Patent Application Publication
(10) Pub. No.: US 2025/0387480 A1
(43) Pub. Date: Dec. 25, 2025

(54) METHODS AND COMPOSITIONS FOR TREATING INFLAMMATORY AND AUTOIMMUNE CONDITIONS WITH ECM-AFFINITY PEPTIDES LINKED TO ANTI-INFLAMMATORY AGENTS

(71) Applicant: The University of Chicago, Chicago, IL (US)

(72) Inventors: Jeffrey A. HUBBELL, Chicago, IL (US); Kiyomitsu KATSUMATA, Chicago, IL (US); Ako ISHIHARA, Chicago, IL (US); Jun ISHIHARA, Chicago, IL (US); Aslan MANSUROV, Chicago, IL (US)

(73) Assignee: The University of Chicago, Chicago, IL (US)

(21) Appl. No.: 19/263,165

(22) Filed: Jul. 8, 2025

Related U.S. Application Data
(62) Division of application No. 17/310,802, filed on Aug. 25, 2021.
(60) Provisional application No. 62/809,988, filed on Feb. 25, 2019.

(51) Int. Cl. A61K 39/44; A61K 38/17; A61K 38/18; A61K 38/19; A61K 38/20; A61K 38/21; A61K 47/64; A61P 37/06

(52) U.S. Cl. CPC A61K 39/44; A61K 38/1709; A61K 38/1774; A61K 38/1841; A61K 38/191; A61K 38/20; A61K 38/2006; A61K 38/2026; A61K 38/2033; A61K 38/2066; A61K 38/2073; A61K 38/215; A61K 47/643; A61P 37/06

(57) ABSTRACT

The disclosure relates to the engineering of collagen-binding modification of anti-inflammatory agents using collagen-binding peptide (CBP) and vWF A3 to achieve targeted therapy for inflammatory diseases. Accordingly, embodiments of the disclosure relate to a composition comprising an anti-inflammatory agent operatively linked to an extracellular matrix (ECM)-affinity peptide. Also disclosed are cytokines and anti-inflammatory agents, such as CD200, linked to a serum protein and/or an ECM-affinity peptide. Further aspects of the disclosure relate to a method for treating an autoimmune or inflammatory condition in a subject comprising administering a composition of the disclosure to the subject.

### CROSS-REFERENCE TO RELATED APPLICATIONS

This application is a divisional of U.S. patent application Ser. No. 17/310,802, filed Aug. 25, 2021, which is a national phase application under 35 U.S.C. § 371 of International Application No. PCT/US2020/019668, filed Feb. 25, 2020, which claims the benefit of priority of U.S. Provisional Patent Application No. 62/809,988 filed Feb. 25, 2019, which is hereby incorporated by reference in its entirety.

### SEQUENCE LISTING

The instant application contains a Sequence Listing which has been submitted in XML format and is hereby incorporated by reference in its entirety. Said XML copy, created on Jul. 3, 2025, is named ARCDP0679USD1-Sequence-Listing.xml and is 94,047 bytes in size.

### BACKGROUND

### I. Field of the Invention

The invention generally relates to the field of medicine. More particularly, it concerns compositions and methods involving nucleotide constructs and proteins-including engineered anti-inflammatory agents for targeting inflamed tissues.

### II. Background

Therapies against cytokines and their receptors have dramatically altered outcomes for inflammatory and autoimmune diseases, especially in anti-TNF therapy for rheumatoid arthritis (RA) and inflammatory bowel disease (IBD) (1-5). However, currently approved medications do not completely cure most patients and possibly have significant side effects by suppressing systemic immunity (6-10). To enhance the therapeutic outcome and reduce systemic side effects, efficient drug delivery to the inflamed area is promising therapeutic strategies for such diseases. Inflammatory tissue releases a range of mediators that will induce the enhanced permeability and retention (EPR) effect (11-12). The EPR effect results from loose endothelial junctions allowing extravasation of macromolecules and nonfunctional lymphatics, resulting in prolonged retention of macromolecules within the solid tumors and inflamed tissues (11-15). Unlike tumor tissue, inflammatory tissue has functional lymphatic system to excrete agents from there (14-16). Currently, there is no effective way to target inflamed tissues of inflammatory and autoimmune diseases due to rapid clearance from the inflamed tissues. Therefore, there is a need in the art for therapies that directly target the inflamed tissues.

### SUMMARY OF INVENTION

The disclosure relates to the engineering of collagen-binding modification of anti-inflammatory agents using collagen-binding peptide (CBP) and vWF A3 to achieve targeted therapy for inflammatory diseases. Accordingly, embodiments of the disclosure relate to a composition comprising an anti-inflammatory agent operatively linked to an extracellular matrix (ECM)-affinity peptide. Aspects of the disclosure also relate to an anti-inflammatory agent operatively linked to a serum protein and compositions containing an anti-inflammatory agent operatively linked to a serum protein. Further aspects of the disclosure relate to a method for treating an autoimmune or inflammatory condition in a subject comprising administering a composition of the disclosure to the subject.

Further aspects of the disclosure relate to a method for reducing inflammation in a subject comprising administering a composition comprising an anti-inflammatory agent operatively linked to an extracellular matrix (ECM)-affinity peptide to the subject. In some embodiments, the inflammation is due to and autoimmune or inflammatory condition and wherein the autoimmune or inflammatory condition comprises inflammatory bowel disease, idiopathic pulmonary fibrosis, multiple sclerosis, type 1 diabetes, or arthritis.

In some embodiments, the anti-inflammatory agent operatively linked to an ECM-affinity peptide comprises a collagen binding domain conjugated to anti-TNFα. In some embodiments, the anti-inflammatory agent operatively linked to an ECM-affinity peptide comprises vWF-A3 operatively linked to IL-4. In some embodiments, the anti-inflammatory agent operatively linked to an ECM-affinity peptide comprises a collagen binding domain conjugated to anti-TGF-β. In some embodiments, the composition is administered systemically. In some embodiments, the composition is administered locally. In some embodiments, the administered dose of the anti-inflammatory agent operatively linked to the ECM-affinity peptide is at least 20% less than the minimum effective dose of the anti-inflammatory agent administered locally without the peptide.

In some embodiments, the anti-inflammatory agent comprises an anti-inflammatory antibody. In some embodiments, the anti-inflammatory antibody comprises an antibody that is specific for TNF-α, IL-1, IL-5, IL-6, IL-6R, IL-12, IL-17A, IL-18, IFN-γ, GM-CSF, CD3, CD20, VLA-4, VLA-5, VCAM-1, TGF-β1, α₄-integrin, α₄β₇-integrin, connective tissue growth factor, platelet-derived growth factor, plasminogen activator inhibitor-1, or insulin-like growth factor-binding protein. In some embodiments, the antibody is an anti-TNF-α, anti-IL-1, anti-IL-5, anti-IL-6, anti-IL-6R, anti-IL-12, anti-IL-17A, anti-IL-18, anti-IFN-γ, anti-GM-CSF, anti-CD3, anti-CD20, anti-VLA-4, anti-VLA-5, anti-VCAM-1, anti-TGF-β1, anti-α₄-integrin, anti-α₄β₇-integrin, anti-connective tissue growth factor, anti-platelet-derived growth factor, anti-plasminogen activator inhibitor-1, or anti-insulin-like growth factor-binding protein antibody. In some embodiments, the anti-inflammatory antibody is a blocking antibody. In some embodiments, the anti-inflammatory antibody is a neutralizing antibody. In some embodiments, the anti-inflammatory antibody is an antagonistic antibody. One or more of these antibodies may be specifically excluded from an embodiment.

In some embodiments, the anti-inflammatory agent comprises an antigen-binding fragment of anti-TNF-α, anti-IL-1, anti-IL-5, anti-IL-6, anti-IL-6R, anti-IL-12, anti-IL-17A, anti-IL-18, anti-IFN-γ, anti-GM-CSF, anti-CD3, anti-CD20, anti-VLA-4, anti-VLA-5, anti-VCAM-1, anti-TGF-β1, anti-α₄-integrin, anti-α₄β₇-integrin, anti-connective tissue growth factor, anti-platelet-derived growth factor, anti-plasminogen activator inhibitor-1, or anti-insulin-like growth factor-binding protein antibody. The antigen binding fragment may comprise a variable light chain region comprising CDR1, CDR2, and CDR3 from an anti-TNF-α, anti-IL-1, anti-IL-5, anti-IL-6, anti-IL-6R, anti-IL-12, anti-IL-17A, anti-IL-18, anti-IFN-γ, anti-GM-CSF, anti-CD3, anti-CD20, anti-VLA-4, anti-VLA-5, anti-VCAM-1, anti-TGF-β1, anti-α₄-integrin, anti-α₄β₇-integrin, anti-connective tissue growth factor, anti-platelet-derived growth factor, anti-plasminogen activator inhibitor-1, or anti-insulin-like growth factor-binding protein antibody and/or a variable heavy chain region comprising CDR1, CDR2, and CDR3 from an anti-TNF-α, anti-IL-1, anti-IL-5, anti-IL-6, anti-IL-6R, anti-IL-12, anti-IL-17A, anti-IL-18, anti-IFN-γ, anti-GM-CSF, anti-CD3, anti-CD20, anti-VLA-4, anti-VLA-5, anti-VCAM-1, anti-TGF-β1, anti-α₄-integrin, anti-α₄β₇-integrin, anti-connective tissue growth factor, anti-platelet-derived growth factor, anti-plasminogen activator inhibitor-1, or anti-insulin-like growth factor-binding protein antibody. In some embodiments the antibody comprises adalimumab, certolizumab, infliximab, golimumab, tocilizumab, rituximab, ustekinumab, natalizumab, vedolizumab, secukinumab, or ixekizumab. In some embodiments, the anti-inflammatory agent comprises an antigen binding fragment derived from adalimumab, certolizumab, infliximab, golimumab, tocilizumab, rituximab, ustekinumab, natalizumab, vedolizumab, secukinumab, or ixekizumab. The antigen binding fragment may comprise a variable light chain region comprising CDR1, CDR2, and CDR3 from adalimumab, certolizumab, infliximab, golimumab, tocilizumab, rituximab, ustekinumab, natalizumab, vedolizumab, secukinumab, or ixekizumab and/or a variable heavy chain region comprising CDR1, CDR2, and CDR3 from adalimumab, certolizumab, infliximab, golimumab, tocilizumab, rituximab, ustekinumab, natalizumab, vedolizumab, secukinumab, or ixekizumab. Examples of antigen binding fragments derived from whole antibodies include minibodies, scFv, chimeric antigen receptors, and diabodies. Also contemplated are bivalent or multispecific constructs derived from one or more of an anti-TNF-α, anti-IL-1, anti-IL-5, anti-IL-6, anti-IL-6R, anti-IL-12, anti-IL-17A, anti-IL-18, anti-IFN-γ, anti-GM-CSF, anti-CD3, anti-CD20, anti-VLA-4, anti-VLA-5, anti-VCAM-1, anti-TGF-β1, anti-α₄-integrin, anti-α₄β₇-integrin, anti-connective tissue growth factor, anti-platelet-derived growth factor, anti-plasminogen activator inhibitor-1, or anti-insulin-like growth factor-binding protein antibody. In some embodiments, the antibody is humanized. In some embodiments, the antibody is a chimeric antibody. One or more of these antibodies or antigen binding fragments may be specifically excluded from an embodiment.

In some embodiments, the antibody comprises an anti-TNF-α antibody. In some embodiments, the antibody comprises an anti-IL-1 antibody. In some embodiments, the antibody comprises an anti-IL-5 antibody. In some embodiments, the antibody comprises an anti-IL-6 antibody. In some embodiments, the antibody comprises an anti-IL-6R antibody. In some embodiments, the antibody comprises an anti-IL-12 antibody. In some embodiments, the antibody comprises an anti-IL-17A antibody. In some embodiments, the antibody comprises an anti-IL-18 antibody. In some embodiments, the antibody comprises an anti-IFN-γ antibody. In some embodiments, the antibody comprises an anti-GM-CSF antibody. In some embodiments, the antibody comprises an anti-CD3 antibody. In some embodiments, the antibody comprises an anti-CD20 antibody. In some embodiments, the antibody comprises an anti-VLA-4 antibody. In some embodiments, the antibody comprises an anti-VLA-5 antibody. In some embodiments, the antibody comprises an anti-VCAM-1 antibody. In some embodiments, the antibody comprises an anti-TGF-β1 antibody. In some embodiments, the antibody comprises an anti-α₄-integrin antibody. In some embodiments, the antibody comprises an anti-α₄β₇-integrin antibody. In some embodiments, the antibody comprises an anti-connective tissue growth factor antibody. In some embodiments, the antibody comprises an anti-platelet-derived growth factor antibody. In some embodiments, the antibody comprises an anti-plasminogen activator inhibitor-1 antibody. In some embodiments, the antibody comprises an anti-insulin-like growth factor-binding protein antibody.

In some embodiments, the anti-inflammatory agent comprises an anti-inflammatory cytokine polypeptide. In some embodiments, the cytokine polypeptide comprises a polypeptide from IL-4, IL-1ra, IL-5, IL-10, IL-11, IL-23, IL-35, IL-36ra, IL-37, interferon-β, TGF-β1, TNF receptor I, and TNF receptor II. In some embodiments, the cytokine polypeptide derived from a human cytokine polypeptide. In some embodiments, the cytokine polypeptide derived from a non-human cytokine polypeptide. In some embodiments, the cytokine polypeptide derived from a mouse, dog, horse, pig, or goat cytokine polypeptide. In some embodiments, the cytokine polypeptide comprises an effector region from one or more of IL-4, IL-1ra, IL-5, IL-10, IL-11, IL-23, IL-35, IL-36ra, IL-37, interferon-β, TGF-β1, TNF receptor I, and TNF receptor II. In some embodiments, the cytokine polypeptide comprises a polypeptide from IL-4. In some embodiments, the cytokine polypeptide comprises a polypeptide from IL-1ra. In some embodiments, the cytokine polypeptide comprises a polypeptide from IL-5. In some embodiments, the cytokine polypeptide comprises a polypeptide from IL-10. In some embodiments, the cytokine polypeptide comprises a polypeptide from IL-11. In some embodiments, the cytokine polypeptide comprises a polypeptide from IL-23, IL-35. In some embodiments, the cytokine polypeptide comprises a polypeptide from IL-36ra. In some embodiments, the cytokine polypeptide comprises a polypeptide from IL-37. In some embodiments, the cytokine polypeptide comprises a polypeptide from interferon-β. In some embodiments, the cytokine polypeptide comprises a polypeptide from TGF-1. In some embodiments, the cytokine polypeptide comprises a polypeptide from TNF receptor I. In some embodiments, the cytokine polypeptide comprises a polypeptide from TNF receptor II. One or more of these anti-inflammatory polypeptides may be specifically excluded from an embodiment. In some embodiments, the cytokine polypeptide is a human cytokine polypeptide or derived from a human cytokine polypeptide.

In some embodiments, the cytokine polypeptide comprises a polypeptide of SEQ ID NO: 18-44 or a fragment thereof or a polypeptide with at least 70, 71, 72, 73, 74, 75, 76, 77, 78, 79, 80. 81, 82, 83, 84, 85, 86, 87, 88, 89, 90, 91, 92, 93, 94, 95, 96, 97, 98, 99, or 100% sequence identity (or any derivable range therein) to a polypeptide having an amino acid sequence of one of SEQ ID NO:18-44 or a fragment thereof. In some embodiments, the anti-inflammatory agent comprises a polypeptide of SEQ ID NO:58 or 59, or fragments thereof or a polypeptide with at least 70, 71, 72, 73, 74, 75, 76, 77, 78, 79, 80. 81, 82, 83, 84, 85, 86, 87, 88, 89, 90, 91, 92, 93, 94, 95, 96, 97, 98, 99, or 100% sequence identity (or any derivable range therein) to a polypeptide having an amino acid sequence of one of SEQ ID NO:58 or 59, or a fragment thereof.

In some embodiments, the anti-inflammatory agent comprises a polypeptide from CD200. In some embodiments, the CD200 polypeptide comprises the extracellular domain of CD200. CD200 (UniProt identifier O54901) is a type-I transmembrane protein that can exert immunosuppressive functions through interaction with its receptor, CD200R1. When cleaved from the surface of the cell, the soluble extracellular domain of CD200 can still bind to and activate CD200R. Embodiments of the disclosure relate to polypeptides comprising at least or at most the extracellular portion of CD200 and a serum protein, such as serum albumin. The polypeptides are useful in the method embodiments of the disclosure. Further embodiments relate to a polypeptide comprising at least or at most the extracellular portion of CD200, serum albumin, and an ECM-affinity polypeptide.

In some embodiments, the ECM-affinity peptide comprises a collagen binding domain. In some embodiments, the polypeptide comprises a collagen binding domain from decorin or von Willebrand factor (VWF). In some embodiments, the ECM-affinity peptide comprises a peptide from placenta growth factor-2 (PIGF-2) or CXCL-12y. In some embodiments, the ECM-affinity peptide comprises a peptide that is at least 85% identical to one of SEQ ID NOS: 1-17, 47, or 52 or a peptide that is at least 85% identical to a fragment of one of SEQ ID NOS: 1-17, 47, or 52. In some embodiments, the ECM-affinity peptide comprises a peptide that has at least 70, 71, 72, 73, 74, 75, 76, 77, 78, 79, 80. 81, 82, 83, 84, 85, 86, 87, 88, 89, 90, 91, 92, 93, 94, 95, 96, 97, 98, 99, or 100% sequence identity (or any derivable range therein) with a peptide having an amino acid sequence of one of SEQ ID NOS: 1-17, 47, or 52 or a peptide that has at least 70, 71, 72, 73, 74, 75, 76, 77, 78, 79, 80. 81, 82, 83, 84, 85, 86, 87, 88, 89, 90, 91, 92, 93, 94, 95, 96, 97, 98, 99, or 100% sequence identity (or any derivable range therein) with a fragment of a peptide having an amino acid sequence of one of SEQ ID NOS: 1-17, 47, or 52.

In some embodiments, the anti-inflammatory agent operatively linked to an extracellular matrix (ECM)-affinity peptide further comprises a serum protein operatively linked to the peptide or agent. In some embodiments, the serum protein is operatively linked to the peptide. In some embodiments, the serum protein is operatively linked to the peptide through a peptide bond. In some embodiments, the serum protein comprises albumin. In some embodiments, the anti-inflammatory agent is amino-proximal to the serum protein. In some embodiments, the anti-inflammatory agent is carboxy-proximal to the serum protein. In some embodiments, the ECM-affinity peptide is amino-proximal to the anti-inflammatory agent. In some embodiments, the ECM-affinity peptide is carboxy-proximal to the anti-inflammatory agent. In some embodiments, the serum protein is amino-proximal to the ECM-affinity peptide. In some embodiments, the serum protein is carboxy-proximal to the ECM-affinity peptide.

A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise. In some embodiments, the composition comprises a collagen binding domain amino-amino proximal to a serum albumin protein, and an IL-10 polypeptide carboxy proximal to the serum albumin protein.

In some embodiments, the peptide is covalently linked to the anti-inflammatory agent and/or other molecules, such as a serum protein. In some embodiments, the peptide is crosslinked to the anti-inflammatory agent through a bifunctional linker. Linkers, such as amino acid or peptidomimetic sequences may be inserted between the peptide and/or antibody sequence. In an embodiment, a fynomer domain is joined to a Heavy (H) chain or Light (L) chain immediately after the last amino acid at the amino (NH₂)-terminus or the carboxy (C)-terminus of the Heavy (H) chain or the Light (L) chain. Linkers may have one or more properties that include a flexible conformation, an inability to form an ordered secondary structure or a hydrophobic or charged character which could promote or interact with either domain. Examples of amino acids typically found in flexible protein regions may include Gly, Asn and Ser. For example, a suitable peptide linker may be GGGSGGGS (SEQ ID NO:48) or (GGGS) n (SEQ ID NO:49), wherein n=1, 2, 3, 4, 5, 6, 7, 8, 9, or 10 (or any range derivable therein). Other near neutral amino acids, such as Thr and Ala, may also be used in the linker sequence. The length of the linker sequence may vary without significantly affecting the function or activity of the fusion protein (see, e.g., U.S. Pat. No. 6,087,329). In a particular aspect, a peptide and an antibody heavy or light chain are joined by a peptide sequence having from about 1 to 25 amino acid residues. Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as CN (where N=1-100 carbon atoms, e.g., N=2, 3, 4, 5, 6, 7, 8, 9, or 10). In some embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some embodiments, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus). Further, the linker may be maleimidocaproyl (mc). In some embodiments, the peptide is linked to the anti-inflammatory agent through a peptide bond. The peptide may be linked to the amino or carboxy terminus of the anti-inflammatory agent. In some embodiments, the peptide is linked to the heavy chain of an anti-inflammatory antibody. In some embodiments, the peptide is linked to the light chain of an anti-inflammatory antibody. In some embodiments, the ratio of peptide to the anti-inflammatory agent is about 1:1 to 5:1. In some embodiments, the ratio of peptide to the anti-inflammatory agent is about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1 (or any derivable range therein). One or more of these linkers may be specifically excluded from an embodiment.

In some embodiments, the composition further comprises a second anti-inflammatory agent operatively linked to an extracellular matrix (ECM)-affinity peptide. In some embodiments, the composition further comprises a third, fourth, fifth, or sixth anti-inflammatory agent operatively linked to an extracellular matrix (ECM)-affinity peptide.

In some embodiments, the autoimmune or inflammatory condition comprises inflammatory bowel disease, idiopathic pulmonary fibrosis, multiple sclerosis, type 1 diabetes, Crohn's disease, psoriasis, acute inflammation, chronic inflammation, neuroinflammation, arthritis, rheumatoid arthritis, fibrosis, infection, allergy, inflammatory therapy-related adverse events, and -related inflammatory illness. One or more of these conditions may be specifically excluded from an embodiment.

In some embodiments, the composition is administered systemically. In some embodiments, the composition is administered by intravenous injection. In some embodiments, the composition is administered locally. In some embodiments, the composition is administered to or adjacent to a site of inflammation.

In some embodiments, the administered dose of the composition comprising the anti-inflammatory agent operatively linked to the peptide is less than the minimum effective dose of the anti-inflammatory agent administered without the peptide. In some embodiments, the administered dose of the composition comprising the anti-inflammatory agent operatively linked to the peptide is less than the minimum effective dose of the anti-inflammatory agent administered without the peptide by the same route of administration. In some embodiments, the administered dose of the anti-inflammatory agent operatively linked to the peptide is at least 10% less than the minimum effective dose of the anti-inflammatory agent administered without the peptide. In some embodiments, the administered dose of the anti-inflammatory agent operatively linked to the peptide is at least 10, 15, 20, 25, 30, 35, 40, 45, 50, 55, 60, 65, or 70% less (or any range derivable therein) than the minimum effective dose of the anti-inflammatory agent administered without the peptide.

In some embodiments, the subject has been previously treated with an anti-inflammatory agent, anti-inflammatory therapy, or autoimmune therapy. In some embodiments, the subject has been determined to be non-responsive to the previous treatment. In some embodiments, the subject has not been treated previously for the inflammatory or autoimmune disease. In some embodiments, the method further comprises administration of an additional inflammatory or autoimmune therapy. In some embodiments, the method further comprises administration of a second anti-inflammatory agent operatively linked to an extracellular matrix (ECM)-affinity peptide.

The term “cytokine polypeptide” as used herein refers to a polypeptide, which is cytokine or a receptor binding domain thereof and retains at a portion of cytokine activity.

The terms “protein”, “polypeptide” and “peptide” are used interchangeably herein when referring to a gene product comprising a polymer of amino acids.

The terms “subject,” “mammal,” and “patient” are used interchangeably. In some embodiments, the subject is a mammal. In some embodiments, the subject is a human. In some embodiments, the subject is a mouse, rat, rabbit, dog, donkey, or a laboratory test animal such as fruit fly, zebrafish, etc.

It is contemplated that the methods and compositions include exclusion of any of the embodiments described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B. CBP-conjugation provided collagen affinity to αTNF. (A) WT-αTNF and CBP-αTNF analyzed by MALDI-TOF MS. Abscissa is mass to charge ratio (m/z) and ordinate is intensity of doubly charged ions. (B) WT-αTNF and CBP-αTNF binding affinities to collagen types I, II, and III are analyzed by ELISA (n=3, mean+SD).

FIG. 2A-D. CBP-αTNF accumulated in the inflamed paw. Arthritis (CAIA) was induced selectively in right hind paw by passive immunization of anti-collagen antibodies, followed by subcutaneous injection of LPS at right hind footpad and PBS at left hind footpad. On the following day of LPS injection, Cy7 labeled CBP-αTNF and Cy7 labeled WT-αTNF were intravenously injected into naïve and CAIA mice. Representative images of accumulation in arthritic or non-arthritic paws of mice injected with CBP-αTNF (A) and WT-αTNF (B). (C) Changes in radiant efficiency ratio of arthritic paw (right hind) to non-arthritic paw (left hind) in naïve and CAIA mice (n=3-4, mean±SD). (D) Representative histology images of joints in CBP-αTNF-injected CAIA mouse (left, H&E staining; right, immunohistochemistry staining against anti-rat IgG).

FIG. 3A-B. CBP-αTNF suppressed arthritis development more effectively than WT-αTNF. Arthritis was induced by passive immunization of anti-collagen antibodies, followed by intraperitoneal injection of LPS. On the day of LPS injection, control IgG, WT-αTNF, or CBP-αTNF was injected intravenously into the arthritis mice. (A) Arthritis scores represent the mean±SE from six mice. * P<0.05, compared with control (Dunnett's multiple comparison test). #P<0.05, compared with the scores on day 8 of each treatment group (Tukey's multiple comparison test). (B) Representative H&E image of joints on day 8 in each treatment group. The severity of synovial hyperplasia and bone resorption was scored 0 to 4 as described in Materials and Method. Statistical analysis was performed using the Dunnett's multiple comparison test for the difference between control and the αTNF treatment groups.

FIG. 4A-B. Subcutaneous injection of CBP-αTNF also accumulated in the arthritic paw and suppressed arthritis development. (A) Arthritis was induced selectively in right hind paw by passive immunization of anti-collagen antibodies, followed by subcutaneous injection of LPS at right hind footpad and PBS at left hind footpad. On the following day of LPS injection, Cy7 labeled CBP-αTNF was subcutaneously injected at the back of the mouse. Representative images of accumulation in arthritic or non-arthritic paws of mice injected with CBP-αTNF (indicated by arrows). (B) Arthritis was induced in all paws by passive immunization of anti-collagen antibodies, followed by intraperitoneal injection of LPS. On the day of LPS injection, control IgG, WT-αTNF, or CBP-αTNF was subcutaneously injected. Arthritis scores represent the mean±SE from five mice. #P<0.05, compared with the scores on day 8 of each group (Tukey's multiple comparison test).

FIG. 5A-B. Effect of local injection on arthritis development. Arthritis was induced by passive immunization of anti-collagen antibodies, followed by intraperitoneal injection of LPS. On the day of LPS injection, (A) Cy7 labeled PIGF-2123-144-αTNF and Cy7 labeled WT-αTNF or (B) control IgG, WT-αTNF, and PIGF-2123-144-αTNF were injected subcutaneously into left hind paw of the arthritis mice. (A) Representative images of retention in the injected site of mice with WT-αTNF and PIGF-2₁₂₃₋₁₄₄-αTNF. (B) Arthritis scores represent the mean±SE from eight mice. #P<0.05, compared with the scores on day 6 of each group (Tukey's multiple comparison test).

FIG. 6A-E. A3-IL4 accumulated in spinal cord and reduced EAE score. Affinity of A3-IL4 against collagen type III (A) and affinity between IL-4 and A3-IL4 protein against IL-4Ra (B) were measured by ELISA. Graph with [concentrations] vs [signals] is shown (n=4). EAE was induced by subcutaneous injection of MOG35-55/CFA emulsion, followed by intraperitoneal injections of PTX on the day of immunization and the following day. On day 14 post-immunization, DyLight 800-labeled A3-IL4, A3 protein (C) or Cy7 labeled CBP-αTNF (D) was intravenously injected into naive or EAE mice. Spinal cord was harvested 4 hours after the injection and fluorescent intensity was measured. (E) From day 14 after the EAE immunization, normal form IL-4 or A3-IL4 was injected intravenously every other day. Disease scores of EAE represent the mean±SE (n=3-5).

FIG. 7A-C. Localization of A3 protein and CBP-conjugate in inflamed tissues of other inflammatory disease models. (A) DyLight 800-labeled A3 protein, or Cy7 labeled CBP-αTNF was intravenously injected into IL-10^{-/-}× TLR-4^{-/-}(DKO) mice that spontaneously developed or non-developed IBD, or normal (C57BL/6) mice. Colon was harvested 4 hours after the injection, and was provided for fluorescent imaging (upper) and histological analyses (lower). Colon of IBD-developed mouse injected Cy7 labeled CBP-αTNF was stained with H&E and periodic acid-Schiff (PAS). In addition, the injected antibody was detected by immunohistochemistry (IHC) against anti-rat IgG. (B) Cy7 labeled CBP-αTGF or Cy7 labeled αTGF was intravenously injected into naïve mice or bleomycin-induced pulmonary fibrosis model 7 days after the bleomycin instillation. Lung was harvested 4 hours after the fluorescence injection, then fluorescent intensity was measured. (C) DyLight 800-labeled A3 protein was intravenously injected into type I diabetes (T1D) spontaneously developed mouse, cyclophosphamide-induced TID mouse, and non-diabetic mouse. Pancreas was harvested 15 minutes after the fluorescence injection, then fluorescent intensity was measured.

FIG. 8A-D. Albumin fusion to IL-10 provided FcRn binding and resulted in LN accumulation. (A) SDS-PAGE analysis for wt IL-10 and SA-IL-10. (B) Binding analysis of SA-IL-10 to FcRn. (C) Splenocytes (i) or single cells from the popliteal LN (ii) were incubated with SA, SA-IL-10 or CBD-SA-IL-10 for 30 min on ice. Shown in (i) for each cell type (x axis) is, from left to right, a bar representing the % binding (y-axis) for SA, SA-IL-10, and CBD-SA-IL-10. Shown in (ii) for each cell type (x axis) is, from left to right, a bar representing the % binding (y-axis) for SA and SA-IL-10, respectively. Binding of each protein to immune cells was detected by co-staining with an anti-SA antibody and antibodies for specific markers of each immune cell population. (D) Immunofluorescence images of the popliteal LN after intravenous injection of DyLight594-labeled wt IL-10 or SA-IL-10. T cells and high endothelial venules (HEVs) were respectively stained with anti-CD3 or anti-PNAd antibodies.

FIG. 9A-B. Albumin fusion to IL-10 provided prolonged blood circulation, and CBD fusion improved biodistribution to the inflamed joint. (A) wt IL-10, SA-IL-10, or CBD-SA-IL-10 (each equivalent to 35 μg of IL-10) were administered to BALB/c mice via tail vein injection. Serum was collected at the indicated time points. The serum concentration of IL-10 was measured by ELISA (mean±SEM; n=5). Plasma half-lives of IL-10 were calculated using two-phase exponential decay: MFI (t)=Ae^{-αt} +Be−^{βt}. t_{1/2, α}, fast clearance half-life; t_{1/2, β}, slow clearance half-life. Area under curve (AUC) was analyzed by Graphpad Prism. (B) Arthritis (CAIA) was induced selectively in the right hind paw by passive immunization of anti-collagen antibodies, followed by subcutaneous injection of LPS at right hind footpad (defined as Day 3). On the day following LPS injection, DyLight800-labeled wt IL-10, SA-IL-10 or CBD-SA-IL-10 were intravenously injected in the CAIA mice. Four hours after injection, indicated organs were harvested and analyzed using an IVIS imaging system. (mean±SEM; n=4). Statistical analyses were done using analysis of variance (ANOVA) with Tukey's test. * P<0.05; ** P<0.01. Shown in (B) for each organ (x axis) is, from left to right, a bar representing the % distribution (y-axis) for wt IL-10, SA-IL-10, and CBD-SA-IL-10, respectively.

FIG. 10A-D. Albumin-fused IL-10 suppressed arthritis development more effectively than wt IL-10. (A) Arthritis (CAIA) was induced by passive immunization with anti-collagen antibodies, followed by intraperitoneal injection of LPS. On the day of LPS injection, PBS, wt IL-10, SA-IL-10 or CBD-SA-IL-10 (equivalent to 43.5 ug of IL-10) was injected intravenously into the arthritic mice. Arthritis scores represent the mean±SEM from 7 mice. (B) Comparison of therapeutic effects of wt IL-10 and CBD-SA-IL-10 with αTNF-α antibody in CAIA mice immunized with high dose (1.5 mg/mouse) of anti-collagen antibodies. Arthritis scores represent the mean±SEM from 6-7 mice. (C) Representative H&E histological image of joints on day 13 in each treatment group. Scale bar, 500 μm. The severity of synovial hyperplasia and bone resorption was scored 0 to 4 as described in Materials and Methods. (D) Effect of administration routes on therapeutic effects of SA-IL-10 and CBD-SA-IL-10. Arthritis scores represent the mean+SEM from 6-7 mice. Statistical analyses were done using analysis of variance (ANOVA) with Tukey's test. * P<0.05; ** P<0.01; *** P<0.001; **** P<0.0001.

FIG. 11A-D. Albumin-fused IL-10 showed improved therapeutic effect on established arthritis. DBA/1J male mice were subcutaneously injected with bovine collagen/CFA emulsion in the tail base. After three weeks, bovine collagen/IFA emulsion was further injected as a boost. When arthritis scores become 2-4 (defined as Day 0), mice were intravenously injected with PBS, SA-IL-10, or CBD-SA-IL-10 (each equivalent to 43.5 μg of IL-10), or with 200 μg of anti-TNF-a antibody. In (A), the same treatments were additionally injected to the mice on Day 3. (A and B) Arthritis scores represent the mean±SEM from 9 mice. (C and D) Representative H&E histological image of joints on day 16. Scale bars, 500 μm. The severity of synovial hyperplasia and bone resorption was scored 0 to 4 as described in Materials and Methods. Statistical analyses were done using analysis of variance (ANOVA) with Tukey's test for (A and B) and a two-tailed Student's t-test for (C and D). * P<0.05; ** P<0.01; *** P<0.001.

FIG. 12A-F. Albumin-fused IL-10 accumulated within and suppressed Th17 activation in LNs. Arthritis (CAIA) was induced by passive immunization of anti-collagen antibodies, followed by intraperitoneal injection of LPS (defined as Day 3). On the day LPS injection, wt IL-10, SA-IL-10 or CBD-SA-IL-10 were intravenously injected into the arthritic mice. IL-10 levels and Th17-relating cytokines in LNs were measured using ELISA. (A) Comparison of IL-10 levels 4 hr after injection of each protein. (B) Pharmacokinetics of wt IL-10 or SA-IL-10 in LNs after intravenous injection. (mean±SEM; n=4) (C) AUC of wt IL-10 and SA-IL-10 in various LNs. Th17-relating cytokine levels in joint-draining (popliteal) LN (D) and a non-draining (cervical) LN (E). (F) GM-CSF levels in the popliteal LN. (mean±SEM; n=7) Statistical analyses were done using analysis of variance (ANOVA) with Tukey's test. * P<0.05; ** P<0.01; *** P<0.001; **** P<0.0001; ns; not significant.

FIG. 13A-C. Albumin-fused IL-10 suppressed inflammatory responses within the paws. Arthritis (CAIA) was induced by passive immunization of anti-collagen antibodies, followed by intraperitoneal injection of LPS. On the day of LPS injection (defined as Day 3), PBS, wt IL-10, SA-IL-10 or CBD-SA-IL-10 were intravenously injected into the arthritic mice. (A) Single cells were extracted from the hind paws on day 11, followed by flow cytometric analysis. Graphs depict the frequency of CD45⁺ cells, B cells (B220⁺ cells within CD45⁺ lymphocytes), dendritic cells (CD11c⁺ cells within CD45⁺ lymphocytes), monocytes (CD11b⁺ cells within CD45⁺ lymphocytes), granulocytic MDSC/neutrophils (Ly6G⁺Ly6C⁺CD11b⁺CD45⁺), monocytic MDSC (Ly6G-Ly6C⁺CD11b⁺CD45⁺), macrophages (F4/80⁺CD11b⁺CD45⁺), M2 macrophages (CD206⁺F4/80⁺CD11b⁺CD45⁺), and M1 macrophages (MHC II⁺F4/80⁺CD11b⁺CD45⁺). (mean±SEM; n=7) (B) Cytokine levels in hind paws on day 11. (n=5-7) (C) Representative H&E images of joints on day 14 in each treatment group. Scale bars, 500 μm. The severity of synovial hyperplasia and bone resorption was scored 0 to 4 as described in Materials and Methods. (mean±SEM; n=6-7). Statistical analyses were done using analysis of variance (ANOVA) with Tukey's test except for % CD11c⁺in (A). For analysis of % CD11c⁺in (A), Kruskal-Wallis test followed by Dunn's multiple comparison test was employed. * P<0.05; ** P<0.01; *** P<0.001; **** P<0.0001.

FIG. 14A-B. CBD-conjugation provided collagen affinity to IL-10. (A) SDS-PAGE analysis for CBD-SA-IL-10. (B) Binding analysis of CBD-SA-IL-10 to type I or type III collagens and FcRn.

FIG. 15A-B. Effect of albumin-fused IL-10 on immune cell populations in the spleen (A) and LNs (B). Arthritis (CAIA) was induced by passive immunization of anti-collagen antibodies, followed by intraperitoneal injection of LPS (defined as Day 3). On Day 3 and Day 6, PBS, wt IL-10, or SA-IL-10 were intravenously injected to mice. Single cells were extracted from the spleen and the popliteal LN on the day following the last injection, followed by flow cytometric analysis. Graphs depict the frequency of CD3⁺T cells within live cells, CD45⁺ lymphocytes within live cells, CD11b⁺ cells within live cells, CD11c⁺ cells within CD11b⁺ cells, CD86⁺ cells within CD11c⁺ cells, granulocytic MDSC/neutrophils (Ly6G⁺Ly6C⁺within CD11b⁺ cells), monocytic MDSC (Ly6G-Ly6C⁺within CD11b⁺ cells), macrophages (F4/80⁺within CD11b⁺ cells), CD86⁺ cells within macrophages, and M2 macrophages (CD206⁺F4/80⁺within CD11b⁺ cells. (mean±SEM; n=6-7) Statistical analyses were done using analysis of variance (ANOVA) with Tukey's test except for the following graphs. For analysis of % CD11c⁺within CD11b⁺ cells in (A) and % Ly6G⁺, Ly6C⁺within CD11b⁺ cells in (B), Kruskal-Wallis test followed by Dunn's multiple comparison test was employed.* P<0.05; ** P<0.01; *** P<0.001; **** P<0.0001.

FIG. 16A-B. Effect of albumin-fused IL-10 on T cell populations in paws and blood. Arthritis (CAIA) was induced by passive immunization of anti-collagen antibodies, followed by intraperitoneal injection of LPS (defined as Day 3). On the day of LPS injection, PBS, wt IL-10, SA-IL-10 or CBD-SA-IL-10 were intravenously injected to mice. (A) Single cells were extracted from the hind paws on day 11, followed by flow cytometric analysis. Graphs depict the frequency of NK1.1⁺CD3-NK cells within CD45⁺ lymphocytes, CD3⁺T cells within CD45⁺ lymphocytes, CD3⁺CD4⁺ T cells within CD45⁺ lymphocytes, Treg (Foxp3⁺CD25⁺) of CD3⁺CD4⁺T cells, CD3⁺CD8⁺ T cells within CD45⁺ lymphocytes, effector memory T cells (CD62L-CD44⁺) of CD3⁺CD8⁺T cells, central memory T cells (CD62L⁺CD44⁺) of CD3⁺CD8⁺T cells, PD-1⁺ cells of CD3⁺CD8⁺T cells. (B) Lymphocytes were extracted from blood on day 11, followed by flow cytometric analysis. Graphs depict the frequency of CD3⁺T cells within CD45⁺ lymphocytes, CD3⁺CD4⁺ T cells within CD45⁺ lymphocytes, Treg (Foxp3⁺CD25⁺) of CD3⁺CD4⁺T cells, CD3⁺CD8⁺ T cells within CD45⁺ lymphocytes. (mean±SEM; n=5-7) Statistical analyses were done using analysis of variance (ANOVA) with Tukey's test except for the following graphs: for analysis of % NK1.1⁺within CD45⁺ cells, % Foxp3⁺within CD4⁺ cells, % CD44⁺/CD62L; within CD8⁺ cells, % CD44⁺/CD62L⁺within CD8⁺ cells and % PD-1⁺within CD8⁺ cells in (A), Kruskal-Wallis test followed by Dunn's multiple comparison test was employed. * P<0.05; ** P<0.01; *** P<0.001.

FIG. 17A-B. Safety assessments of albumin-fused IL-10. wt IL-10, SA-IL-10, or CBD-SA-IL-10 were intravenously injected to healthy BALB/c mice. (A) Two days after injection, white blood cell counts, red blood cell counts, platelet counts, the concentration of hemoglobin in blood and the weight of spleen were assessed. (B) The concentrations of alanine transaminase (ALT), amylase, blood urea nitrogen (BUN), serum calcium, creatine kinase (CK), CO₂, total bilirubin (TBli) and total proteins in serum were assessed using biochemistry analyzer. (mean±SEM; n=5) Statistical analyses were done using analysis of variance (ANOVA) with Tukey's test. * P<0.05; ** P<0.01.

FIG. 18A-D. IL-4 retains activity after fusion of SA. (a) Wt IL-4 and SA-IL-4 were analyzed by SDS-PAGE under reducing and non-reducing conditions with Coomassie blue staining. (b) SA-IL-4 binding to freshly isolated immune cells from LN and spleen, measured by flow cytometry. (c) Wt IL-4 and SA-IL-4 activity assay. Phosphorylation of STAT6 in the T cells was analyzed by flow cytometry after culturing T cells in vitro with indicated concentrations of wt IL-4 or SA-IL-4. (d) IL-17 concentration secreted under Th17 differentiation conditions in the presence of wt IL-4 or SA-IL-4, measured by ELISA. Data are mean±SEM. Two experimental replicates. Statistical analyses were performed using one-way ANOVA with Tukey's test. ** P<0.01.

FIG. 19A-H. SA fusion to IL-4 increased the amount of IL-4 in the secondary lymphoid organs after intravenous injection. (a) Binding affinity of SA-IL-4 to FcRn measured by SPR. (b) Plasma concentration of intravenously injected wt IL-4 or SA-IL-4. Wt IL-4 10 μg (n=4) or equimolar SA-IL-4 (n=3) was injected intravenously to naïve mice. Blood was collected after 1 min to 24 hr, and the plasma concentration of IL-4 was measured by ELISA. (c-d) Amount of IL-4 in the (c) brachial and lumbar LNs and (d) spleen over time. 40 μg of wt IL-4 or equimolar SA-IL-4 was injected intravenously to naïve mice. LNs and spleen were harvested, and IL-4 amount was tested by ELISA (n=5). (e) SA (P573K)-IL-4 amount in the LNs and spleen was measured 1 hr after injection. Wt IL-4 and SA-IL-4 data from (c-d) are re-presented. (f-g) Immunofluorescence images of the lumbar LN, 1 hr after intravenous injection of DyLight594-labeled IL-4 or SA-IL-4. T cells and high endothelial venules (HEVs) were respectively stained by anti-CD3 or anti-PNAd antibodies. Scale bars represent (g) 200 μm and (h) 100 μm. Data are mean±SEM. Two experimental replicates. Statistical analyses were performed using one-way ANOVA with Tukey's test. ** P<0.01.

FIG. 20A-D. SA-IL-4 prevents EAE disease progression and development in the acute phase. (a) Disease progression with (b) disease incidence and (c) body weight change in C57BL/6 myelin oligodendrocyte glycoprotein (MOG) 35-55 experimental autoimmune encephalomyelitis (EAE) mice injected every other day for 10 days from day 8 after immunization with phosphate-buffered saline (PBS) intraperitoneally (i.p.), wt IL-4 10 μg i.p., or SA-IL-4 10 μg molar equivalent i.p or subcutaneously (s.c.), or dosed with FTY720 1 mg/kg by oral administration. n=7 per group. (d) Representative histology of spinal cord. Myelin expression was detected by immunohistochemistry with anti-myelin basic protein antibody (brown). Arrows indicate demyelination. Graph represents % of mice showing demyelination in each treatment group by blinded pathology analysis. Two experimental replicates. Data are mean±SEM. Statistical analyses were performed using one-way ANOVA with Tukey's test. ** P<0.01.

FIG. 21A-H. SA-IL-4 treatment inhibits leukocyte infiltration to the spinal cord and induces immune suppressive cells in the draining LN. Mice were injected with wt IL-4, SA-IL-4, or PBS i.p. or SA-IL-4 s.c. every other day for 10 days from day 8 after immunization, or FTY720 1 mg/kg body weight was administered orally every day from day 8 after immunization. 17 days after immunization, cells from the draining LN and spinal cord were isolated and analyzed by flow cytometry. (a-b) The frequencies of (a) CD45⁺leukocytes and (b) RoR Dt+Th17 cells within live cells in the spinal cord. (c-g) In the lumbar draining LN (dLN), frequencies of (c) Ly6G⁺Ly6C⁺G-MDSCs within CD11b⁺CD45⁺ cells, (d) Ly6G-Ly6C⁺M-MDSCs within CD11b⁺CD45⁺ cells, (e) RoROt+Th17 cells within CD4⁺CD3⁺T cells, (f) CD86⁺M1 macrophages within F4/80⁺CD11b⁺macrophages, (g) CD206⁺M2 macrophages within F4/80⁺CD11b⁺macrophages, and (h) B220⁺B cells within CD11b⁺CD45⁺ cells were analyzed. Data are mean±SEM. The experiment was performed once. Statistical analyses were performed using one-way ANOVA with Tukey's test. * P<0.05, ** P<0.01.

FIG. 22A-P. SA-IL-4 treatment activates the PD-1/PD-L1 axis and decreases integrin and cytokine expression in T cells. MOG35-55-induced EAE mice were injected with PBS, wt IL-4 or SA-IL-4 s.c. on days 8, 10 and 12 after immunization. The spinal cord and spleen were isolated on day 13 and (a-i) immune cells were analyzed. Frequencies of (a) Tetramer⁺ (recognizing MOG35-55) cells within CD4⁺ T cells in the spinal cord. In the spleen, (b) aLB2 integrin⁺ cells within Tetramer⁺CD4⁺T cells, (c) α4B1 integrin⁺ cells within Tetramer⁺CD4⁺T cells, (d) aLB2 integrin⁺ cells within CD8⁺ T cells, and (e) α4B1 integrin⁺ cells within CD8⁺T cells, are shown. (f) Mean fluorescence intensity (MFI) of PD-1 of central memory (CM) CD44⁺CD62L⁺CD4⁺T cells, (g) MFI of PD-1 of CM CD44⁺CD62L⁺CD8⁺T cells, (h) MFI of PD-L1 of Ly6C⁺Ly6G-CD11b⁺M-MDSC, (i) frequency of PD-L1⁺of Ly6C⁺Ly6G-CD11b⁺M-MDSC, (j) MFI of PD-L1 of Ly6C⁺Ly6G⁺CD11b⁺G-MDSC, (k) frequency of PD-L1⁺of Ly6C⁺Ly6G⁺CD11b⁺G-MDSC, (1) frequency of IL-23R⁺ cells within Tetramer⁺CD4⁺T cells. (m-n) Splenocytes were cultured in vitro in the presence of MOG protein for 3 days. (m) IL-17A, (n) IFNγ, (0) GM-CSF concentrations in the culture media were analyzed by ELISA. (p) Splenocytes were cultured in vitro in the presence of MOG35-55 peptide for 6 h. Cytokine expression within CD4⁺T cells was characterized by flow cytometry. Data are mean±SEM. The experiment was performed once. Statistical analyses were performed using one-way ANOVA with Tukey's test. * P<0.05, ** P<0.01.

FIG. 23A-K. SA-IL-4 treatment in the chronic phase of EAE decreases the clinical score and prevents immune cell infiltration to the spinal cord. EAE was induced in C57BL/6 mice using MOG35-55. PBS, wt IL-4 or SA-IL-4 was injected i.p. every other day for 10 days from day 21 after immunization. (a) Disease progression and (b) body weight change are shown (n=6). (c-d) PBS, wt IL-4 or SA-IL-4 was injected s.c. every other day for 12 days from day 21 after immunization. (c) Disease progression and (d) body weight change are shown (n=8 for PBS and SA-IL-4; n=7 for other treatment groups). (e-h) On day 34, the spinal cord and spleen were collected and immune cells were analyzed by flow cytometry. Graphs represent frequencies of (e) CD45⁺ cells within live cells in spinal cord, (f) CD4⁺CD3⁺CD45⁺T cells within live cells in spinal cord, (g) Tetramer⁺ (recognizing MOG₃₅₋₅₅) RoRγt⁺CD4⁺Th17 cells within live cells in spinal cord, (h) IL-23R⁺ cells within Tetramer⁺CD4⁺ cells in spleen. (i-j) Splenocytes were cultured in vitro in the presence of MOG protein for 3 days. (i) IL-17A, (j) GM-CSF concentrations in the culture media were analyzed by ELISA. (k) Splenocytes were cultured in vitro in the presence of MOG₃₅₋₅₅ peptide for 6 h. Cytokine expression within CD4⁺T cells was characterized by flow cytometry. The experiment was performed once. Data are mean±SEM. Statistical analyses were performed using one-way ANOVA with Tukey's test. * P<0.05, ** P<0.01.

FIG. 24. Injected SA-IL-4 accumulates in the LN more than wt IL-4, analyzed by IVIS. Biodistribution analysis of DyLight 800-labeled wt IL-4 and SA-IL-4 in the LN. 10 μg of wt IL-4 or SA-IL-4 with an equal amount of fluorescence was injected i.v . . . 4 hr after injection, the lumbar LN was harvested and imaged by an IVIS in vivo imaging system (n=6). Data are mean±SEM. The experiment was performed once. Statistical analyses were performed using Student's t-test.

FIG. 25A-B. SA (P573K) mutation to SA-IL-4 decreases blood concentration and abolished FcRn binding. Mice were injected with 40 μg of wt IL-4, SA-IL-4, or SA (P573K)-IL-4 i.v . . . . After 1 hr, blood was collected and IL-4 concentration in the plasma was determined by ELISA. Data are mean±SEM. (b) Binding affinity of SA (P573K)-IL-4 and FcRn measured by SPR. The binding affinity could not be determined. 1^{st} and 2^{nd} mean the 62.5 nM concentration was tested twice to validate the variability. Two experimental replicates.

FIG. 26. Long-term treatment of SA-IL-4 suppresses EAE disease development and progression. Disease progression in C57BL/6 MOG₃₅₋₅₅ EAE mice injected i.p. every other day for 16 days from day 8 after immunization with PBS or SA-IL-4 (10 μg on an IL-4 basis) (n=8). The number of mice that developed EAE per total mice in each treatment group is indicated. Mice were monitored until day 24. Data are mean±SEM. Two experimental replicates. Statistical analyses were performed using Student's t-test. ** P<0.01.

FIG. 27, FcRn binding is crucial for SA-IL-4 to suppresses EAE disease development and progression. Disease progression in C57BL/6 MOG₃₅₋₅₅ EAE mice injected i.p. every other day from day 8 after immunization with PBS or SA-IL-4 (10 μg on an IL-4 basis) (n=6). Data are mean±SEM. Two experimental replicates. Statistical analyses were performed using Student's t-test. ** P<0.01.

FIG. 28A-B. SA-IL-4 did not affect the number of macrophages and dendritic cells in the spinal cord and draining LN. Mice were injected with wt IL-4, SA-IL-4, or PBS i.p. or SA-IL-4 s.c. every other day for 10 days from day 8 after immunization. FTY720 1 mg/kg body weight was administered orally every day from day 8 after immunization. 17 days after immunization, cells from the draining LN (dLN) and spinal cord were isolated and analyzed by flow cytometry. The frequencies of (a) F4/80⁺macrophage within CD11b⁺ cells, and (b) CD11b⁺CD11c⁺DCs within CD45⁺ cells were analyzed. Data are mean±SEM. The experiment was performed once. Statistical analyses were performed using one-way ANOVA with Tukey's test.

FIG. 29A-O. Blood and organ analysis of SA-IL-4 revealed SA-IL-4 is safe. Toxicity analysis of SA-IL-4. 10 μg of wt IL-4 or equimolar SA-IL-4 was injected i.v. to naïve mice. After 2 days, (a-i) serum was tested using a biochemistry analyzer and (j-m) blood was tested using a hematology analyzer. Lung water content was determined by weighing the lungs before and after lyophilization. Data are mean±SEM. The experiment was performed once. Statistical analyses were performed using one-way ANOVA with Tukey's test.

FIG. 30A-D. Gating strategy for flow cytometry. (A) Re-stimulation (cytokine expression). (B) Integrin expression. (C) MDSCs. (D) CD45⁺ and T cells.

### DETAILED DESCRIPTION

Enhancing therapeutic efficacy of drugs for inflammatory and autoimmune diseases is of huge demand. One possible approach is that targeting anti-inflammatory drugs to inflamed area. Collagens are not accessible in most tissues due to the low permeability of the vasculature, yet are exposed to the bloodstream in the inflamed area due to the hyperpermeability of the vasculature. This disclosure describes ECM-binding anti-inflammatory agents conjugated to ECM-affinity peptides. One such peptide is a collagen-binding peptide (CBP). CBP-conjugation provided collagen affinity to anti-TNFα antibody (αTNF). CBP-αTNF accumulated in inflamed areas of the collagen antibody-induced arthritis model (Example 1). Arthritis development was significantly suppressed by CBP-αTNF compared with the unmodified antibody. Moreover, collagen-binding domain derived from von Willebrand factor (vWF) A3 domain fusion to interleukin (IL)-4 (A3-IL4) enabled it to be detectable in the spinal cord of the experimental autoimmune encephalomyelitis (EAE), a model for multiple sclerosis, following intravenous administration (Example 1). A3-IL4 reduced the clinical symptoms of EAE whereas normal IL-4 did not. Collagen-binding proteins were detectable in the inflamed tissues of the spontaneous inflammatory bowel disease, bleomycin-induced pulmonary fibrosis, and type I diabetes models. Taken together, collagen-affinity enables the anti-inflammatory drugs to target inflamed areas, demonstrating a novel clinically translational approach to treat inflammatory and autoimmune diseases.

### III. Anti-inflammatory Agents

### A. Antibodies

Aspects of the disclosure relate to anti-inflammatory antibodies or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4:302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the-COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F (ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the V_{L} domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the C_{L} domain. This structure and nomenclature is repeated for the VH chain, which includes three CDRs identified as H1, H2 and H3. The majority of amino acid residues in the variable domains, or Fv fragments (V_{H} and V_{L}), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann.

Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### 1. Functional Antibody Fragments and Antigen-Binding Fragments

a. Antigen-Binding Fragments

Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F (ab′) 2 fragment means a bivalent antigen-binding fragment of a monoclonal antibody comprising two Fab′ fragments linked by a disulfide bridge at the hinge region. An F (ab′) 2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv) 2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens. b. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

2. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### B. Cytokines

Cytokines are a group of proteins that cells release upon excitation (only very few cytokines are expressed on cell membranes). Cytokines produced by cells can affect target cells nearby or through blood circulation at very low concentration. They have broad functions on promoting growth, differentiation, and activation of target cells. Many cytokines can target immune cells and play a role in immune response. Based on structural and functional differences, cytokines may be broadly divided into chemokines, interleukins, growth factors, transforming growth factors, colony stimulating factors, tumor necrosis factors, and interferons.

The following cytokines may be used as anti-inflammatory agents in methods and compositions of the disclosure. While exemplary sequences are provided below, equivalent or homologous proteins known in the art may also be used.

Human IL-1ra:
(SEQ ID NO: 18)
RPSGRKSSKMQAFRIWDVNQKTFYLRNNQLVAGYLQGPNVNLEEKIDVVPIEPHALF
LGIHGGKMCLSCVKSGDETRLQLEAVNITDLSENRKQDKRFAFIRSDSGPTTSFESAA
CPGWFLCTAMEADQPVSLTNMPDEGVMVTKFYFQEDE.
Mouse IL-1ra:
(SEQ ID NO: 19)
RPSGKRPCKMQAFRIWDTNQKTFYLRNNQLIAGYLQGPNIKLEEKIDMVPIDLHSVFL
GIHGGKLCLSCAKSGDDIKLQLEEVNITDLSKNKEEDKRFTFIRSEKGPTTSFESAACP
GWFLCTTLEADRPVSLTNTPEEPLIVTKFYFQEDQ
Human IL-4:
(SEQ ID NO: 20)
HKCDITLQEIIKTLNSLTEQKTLCTELTVTDIFAASKNTTEKETFCRAATVLRQFYSHH
EKDTRCLGATAQQFHRHKQLIRFLKRLDRNLWGLAGLNSCPVKEANQSTLENFLER
LKTIMREKYSKCSS.
Mouse IL-4:
(SEQ ID NO: 21)
HIHGCDKNHLREIIGILNEVTGEGTPCTEMDVPNVLTATKNTTESELVCRASKVLRIF
YLKHGKTPCLKKNSSVLMELQRLFRAFRCLDSSISCTMNESKSTSLKDFLESLKSIMQ
MDYS
Human IL-5:
(SEQ ID NO: 22)
IPTEIPTSALVKETLALLSTHRTLLIANETLRIPVPVHKNHQLCTEEIFQGIGTLESQTVQ
GGTVERLFKNLSLIKKYIDGQKKKCGEERRRVNQFLDYLQEFLGVNINTEWIIES.
Mouse IL-5:
(SEQ ID NO: 23)
EIPMSTVVKETLTQLSAHRALLTSNETMRLPVPTHKNHQLCIGEIFQGLDILKNQTVR
GGTVEMLFQNLSLIKKYIDRQKEKCGEERRRTRQFLDYLQEFLGVMSTEWAMEG.
Human IL-10:
(SEQ ID NO: 24)
SPGQGTQSENSCTHFPGNLPNMLRDLRDAFSRVKTFFQMKDQLDNLLLKESLLEDFK
GYLGCQALSEMIQFYLEEVMPQAENQDPDIKAHVNSLGENLKTLRLRLRRCHRFLPC
ENKSKAVEQVKNAFNKLQEKGIYKAMSEFDIFINYIEAYMTMKIRN.
Mouse IL-10:
(SEQ ID NO: 25)
LGCQALSEMIQFYLVEVNIPQAEKHGPEIKEHLNSLGQKLKTLRMRLRRCHRFLPCEN
KSKAVEQVKSDFNKLEDQGVYKAMNEFDIFINCIEAYMMIKMKS.
Human IL-11:
(SEQ ID NO: 26)
PGPPPGPPRVSPDPRAELDSTVLLTRSLLADTRQLAAQLRDKFPADGDHNLDSLPTLA
MSAGALGALQLPGVLTRLRADLLSYLRHVQWLRRAGGSSLKTLEPELGTLQARLDR
LLRRLQLLMSRLALPQPPPDPPAPPLAPPSSAWGGIRAAHAILGGLHLTLDWAVRGL
LLLKTRL.
Mouse IL-11:
(SEQ ID NO: 27)
MPGPPAGSPRVSSDPRADLDSAVLLTRSLLADTRQLAAQMRDKFPADGDHSLDSLPT
LAMSAGTLGSLQLPGVLTRLRVDLMSYLRHVQWLRRAGGPSLKTLEPELGALQARL
ERLLRRLQLLMSRLALPQAAPDQPVIPLGPPASAWGSIRAAHAILGGLHLTLDWAVR
GLLLLKTRL.
Human IL-23; p19 Subunit:
(SEQ ID NO: 28)
RAVPGGSSPAWTQCQQLSQKLCTLAWSAHPLVGHMDLREEGDEETTNDVPHIQCGD
GCDPQGLRDNSQFCLQRIHQGLIFYEKLLGSDIFTGEPSLLPDSPVGQLHASLLGLSQL
LQPEGHHWETQQIPSLSPSQPWQRLLLRFKILRSLQAFVAVAARVFAHGAATLSP.
p40 Subunit:
(SEQ ID NO: 29)
IWELKKDVYVVELDWYPDAPGEMVVLTCDTPEEDGITWTLDQSSEVLGSGKTLTIQ
VKEFGDAGQYTCHKGGEVLSHSLLLLHKKEDGIWSTDILKDQKEPKNKTFLRCEAK
NYSGRFTCWWLTTISTDLTFSVKSSRGSSDPQGVTCGAATLSAERVRGDNKEYEYSV
ECQEDSACPAAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVICRKNASIS
VRAQDRYYSSSWSEWASVPCS.
Human IL-35; p35 Subunit:
(SEQ ID NO: 30)
RNLPVATPDPGMFPCLHHSQNLLRAVSNMLQKARQTLEFYPCTSEEIDHEDITKDKT
STVEACLPLELTKNESCLNSRETSFITNGSCLASRKTSFMMALCLSSIYEDLKMYQVE
FKTMNAKLLMDPKRQIFLDQNMLAVIDELMQALNFNSETVPQKSSLEEPDFYKTKIK
LCILLHAFRIRAVTIDRVMSYLNAS;
Ebi3 subunit:
(SEQ ID NO: 31)
RKGPPAALTLPRVQCRASRYPIAVDCSWTLPPAPNSTSPVSFIATYRLGMAARGHSW
PCLQQTPTSTSCTITDVQLFSMAPYVLNVTAVHPWGSSSSFVPFITEHIIKPDPPEGVRL
SPLAERQLQVQWEPPGSWPFPEIFSLKYWIRYKRQGAARFHRVGPTEATSFILRAVRP
RARYYVQVAAQDLTDYGELSDWSLPATATMSLGK.
Mouse IL-35; p35 Subunit:
(SEQ ID NO: 32)
RVIPVSGPARCLSQSRNLLKTTDDMVKTAREKLKHYSCTAEDIDHEDITRDQTSTLKT
CLPLELHKNESCLATRETSSTTRGSCLPPQKTSLMMTLCLGSIYEDLKMYQTEFQAIN
AALQNHNHQQIILDKGMLVAIDELMQSLNHNGETLRQKPPVGEADPYRVKMKLCIL
LHAFSTRVVTINRVMGYLSSA;
Ebi3 subunit:
(SEQ ID NO: 33)
ALVALSQPRVQCHASRYPVAVDCSWTPLQAPNSTRSTSFIATYRLGVATQQQSQPCL
QRSPQASRCTIPDVHLFSTVPYMLNVTAVHPGGASSSLLAFVAERIIKPDPPEGVRLRT
AGQRLQVLWHPPASWPFPDIFSLKYRLRYRRRGASHFRQVGPTEATTFTLRNSKPHA
KYCIQVSAQDLTDYGKPSDWSLPGQVESAPHKP.
Human IL-36ra:
(SEQ ID NO: 34)
VLSGALCFRMKDSALKVLYLHNNQLLAGGLHAGKVIKGEEISVVPNRWLDASLSPVI
LGVQGGSQCLSCGVGQEPTLTLEPVNIMELYLGAKESKSFTFYRRDMGLTSSFESAA
YPGWFLCTVPEADQPVRLTQLPENGGWNAPITDFYFQQCD.
Mouse IL-36ra:
(SEQ ID NO: 35)
VLSGALCFRMKDSALKVLYLHNNQLLAGGLHAEKVIKGEEISVVPNRALDASLSPVI
LGVQGGSQCLSCGTEKGPILKLEPVNIMELYLGAKESKSFTFYRRDMGLTSSFESAAY
PGWFLCTSPEADQPVRLTQIPEDPAWDAPITDFYFQQCD.
Human IL-37:
(SEQ ID NO: 36)
VHTSPKVKNLNPKKFSIHDQDHKVLVLDSGNLIAVPDKNYIRPEIFFALASSLSSASAE
KGSPILLGVSKGEFCLYCDKDKGQSHPSLQLKKEKLMKLAAQKESARRPFIFYRAQV
GSWNMLESAAHPGWFICTSCNCNEPVGVTDKFENRKHIEFSFQPVCKAEMSPSEVSD.
Mouse IL-37:
(SEQ ID NO: 37)
VPSSAKVNARAPEKFSIRDQDQKVLVLSDETLIAVPNKPYTVPETFFVLASHTSSSHE
GSPILLAVSKGELCLCCDKDEEQSKPSLQLKKNELMKLATQKEKVRLPFVFYRAQVG
SCCTLESAAHPGWFVCTSRNSGAPVEVTDTSGEGKLMEFSFQQVSETEMSPSEVSI.
Human interferon-β:
(SEQ ID NO: 38)
SYNLLGFLQRSSNFQCQKLLWQLNGRLEYCLKDRMNFDIPEEIKQLQQFQKEDAALT
IYEMLQNIFAIFRQDSSSTGWNETIVENLLANVYHQINHLKTVLEEKLEKEDFTRGKL
MSSLHLKRYYGRILHYLKAKEYSHCAWTIVRVEILRNFYFINRLTGYLRN.
Mouse interferon-β:
(SEQ ID NO: 39)
INYKQLQLQERTNIRKCQELLEQLNGKINLTYRADFKIPMEMTEKMQKSYTAFAIQE
MLQNVFLVFRNNFSSTGWNETIVVRLLDELHQQTVFLKTVLEEKQEERLTWEMSST
ALHLKSYYWRVQRYLKLMKYNSYAWMVVRAEIFRNFLIIRRLTRNFQN.
Human TGF-β1:
(SEQ ID NO: 40)
ALDTNYCFSSTEKNCCVRQLYIDFRKDLGWKWIHEPKGYHANFCLGPCPYIWSLDT
QYSKVLALYNQHNPGASAAPCCVPQALEPLPIVYYVGRKPKVEQLSNMIVRSCKCS.
Mouse TGF-β1:
(SEQ ID NO: 41)
ALDTNYCFSSTEKNCCVRQLYIDFRKDLGWKWIHEPKGYHANFCLGPCPYIWSLDT
QYSKVLALYNQHNPGASASPCCVPQALEPLPIVYYVGRKPKVEQLSNMIVRSCKCS.
Human TNF receptor I:
(SEQ ID NO: 42)
DSVCPQGKYIHPQNNSICCTKCHKGTYLYNDCPGPGQDTDCRECESGSFTASENHLR
HCLSCSKCRKEMGQVEISSCTVDRDTVCGCRKNQYRHYWSENLFQCFNCSLCLNGT
VHLSCQEKQNTVCTCHAGFFLRENECVSCSNCKKSLECTKLCLPQIEN.
Human TNF receptor II:
(SEQ ID NO: 43)
LPAQVAFTPYAPEPGSTCRLREYYDQTAQMCCSKCSPGQHAKVFCTKTSDTVCDSC
EDSTYTQLWNWVPECLSCGSRCSSDQVETQACTREQNRICTCRPGWYCALSKQEGC
RLCAPLRKCRPGFGVARPGTETSDVVCKPCAPGTFSNTTSSTDICRPHQICNVVAIPG
NASMDAVCTSTSPTRSMAPGAVHLPQPVSTRSQHTQPTPEPSTAPSTSFLLPMGPSPP
AEGSTGDFALPVGLIVGVTALGLLIIGVVNCVIIVITQVKKKPLCLQREAKVPHLPADK
ARGTQGPEQQHLLITAPSSSSSSLESSASALDRRAPTRNQPQAPGVEASGAGEARAST
GSSDSSPGGHGTQVNVTCIVNVCSSSDHSSQCSSQASSTMGDTDSSPSESPKDEQVPF
SKEECAFRSQLETPETLLGSTEEKPLPLGVPDAGMKPS.
Mouse TNF receptor II:
(SEQ ID NO: 44)
VPAQVVLTPYKPEPGYECQISQEYYDRKAQMCCAKCPPGQYVKHFCNKTSDTVCAD
CEASMYTQVWNQFRTCLSCSSSCTTDQVEIRACTKQQNRVCACEAGRYCALKTHSG
SCRQCMRLSKCGPGFGVASSRAPNGNVLCKACAPGTFSDTTSSTDVCRPHRICSILAI
PGNASTDAVCAPESPTLSAIPRTLYVSQPEPTRSQPLDQEPGPSQTPSILTSLGSTPIIEQ
STKGG.

### C. CD200

Embodiments of the disclosure relate to polypeptides and compositions comprising the anti-inflammatory agent, CD200. Exemplary CD200 polypeptide amino acid sequences are shown below:

Mouse CD200 extracellular domain is represented by the following sequence:
(SEQ ID NO: 58)
QVEVVTQDERKALHTTASLRCSLKTSQEPLIVTWQKKKAVSPENMVTYSKTHGVVIQP
AYKDRINVTELGLWNSSITFWNTTLEDEGCYMCLFNTFGSQKVSGTACLTLYVQPIVHL
HYNYFEDHLNITCSATARPAPAISWKGTGTGIENSTESHFHSNGTTSVTSILRVKDPKTQ
VGKEVICQVLYLGNVIDYKQSLDKG.
Mouse CD200-MSA fusion protein (linker is underlined):
(SEQ ID NO: 66)
QVEVVTQDERKALHTTASLRCSLKTSQEPLIVTWQKKKAVSPENMVTYSKTHGVVIQP
AYKDRINVTELGLWNSSITFWNTTLEDEGCYMCLFNTFGSQKVSGTACLTLYVQPIVHL
HYNYFEDHLNITCSATARPAPAISWKGTGTGIENSTESHFHSNGTTSVTSILRVKDPKTQ
VGKEVICQVLYLGNVIDYKQSLDKGGGGSGGGSEAHKSEIAHRYNDLGEQHFKGLVLIA
FSQYLQKCSYDEHAKLVQEVTDFAKTCVADESAANCDKSLHTLFGDKLCAIPNLRENY
GELADCCTKQEPERNECFLQHKDDNPSLPPFERPEAEAMCTSFKENPTTFMGHYLHEVA
RRHPYFYAPELLYYAEQYNEILTQCCAEADKESCLTPKLDGVKEKALVSSVRQRMKCSS
MQKFGERAFKAWAVARLSQTFPNADFAEITKLATDLTKVNKECCHGDLLECADDRAEL
AKYMCENQATISSKLQTCCDKPLLKKAHCLSEVEHDTMPADLPAIAADFVEDQEVCKN
YAEAKDVFLGTFLYEYSRRHPDYSVSLLLRLAKKYEATLEKCCAEANPPACYGTVLAEF
QPLVEEPKNLVKTNCDLYEKLGEYGFQNAILVRYTQKAPQVSTPTLVEAARNLGRVGT
KCCTLPEDQRLPCVEDYLSAILNRVCLLHEKTPVSEHVTKCCSGSLVERRPCFSALTVDE
TYVPKEFKAETFTFHSDICTLPEKEKQIKKQTALAELVKHKPKATAEQLKTVMDDFAQF
LDTCCKAADKDTCFSTEGPNLVTRCKDALA
Human CD200 extracellular domain (UniProt identifier P41217) is represented by the
following sequence:
(SEQ ID NO: 59)
QVQVVTQDEREQLYTPASLKCSLQNAQEALIVTWQKKKAVSPENMVTFSENHGVVIQP
AYKDKINITQLGLQNSTITFWNITLEDEGCYMCLFNTFGFGKISGTACLTVYVQPIVSLHY
KFSEDHLNITCSATARPAPMVFWKVPRSGIENSTVTLSHPNGTTSVTSILHIKDPKNQVG
KEVICQVLHLGTVTDFKQTVNKG.
An exemplary Human CD200 (lower case) -human serum albumin (upper case) fusion
protein (linker is uppercase and underlined) is represented by the following:
(SEQ ID NO: 61)
qvqvvtqdereqlytpaslkcslqnaqealivtwqkkkavspenmvtfsenhgvviqpaykdkinitqlglqnstitfwnitledegcy
mclfntfgfgkisgtacltvyvqpivslhykfsedhlnitcsatarpapmvfwkvprsgienstvtlshpngttsvtsilhikdpknqvgk
evicqvlhlgtvtdfkqtvnkgGGGSGGGSDAHKSEVAHRFKDLGEENFKALVLIAFAQYLQQCPF
EDHVKLVNEVTEFAKTCVADESAENCDKSLHTLFGDKLCTVATLRETYGEMADCCAK
QEPERNECFLQHKDDNPNLPRLVRPEVDVMCTAFHDNEETFLKKYLYEIARRHPYFYAP
ELLFFAKRYKAAFTECCQAADKAACLLPKLDELRDEGKASSAKQRLKCASLQKFGERA
FKAWAVARLSQRFPKAEFAEVSKLVTDLTKVHTECCHGDLLECADDRADLAKYICENQ
DSISSKLKECCEKPLLEKSHCIAEVENDEMPADLPSLAADFVESKDVCKNYAEAKDVFL
GMFLYEYARRHPDYSVVLLLRLAKTYETTLEKCCAAADPHECYAKVFDEFKPLVEEPQ
NLIKQNCELFEQLGEYKFQNALLVRYTKKVPQVSTPTLVEVSRNLGKVGSKCCKHPEAK
RMPCAEDYLSVVLNQLCVLHEKTPVSDRVTKCCTESLVNRRPCFSALEVDETYVPKEFN
AETFTFHADICTLSEKERQIKKQTALVELVKHKPKATKEQLKAVMDDFAAFVEKCCKA
DDKETCFAEEGKKLVAASQAALGL
Mouse CD200 (lower case)-CBD fusion protein (upper case) (linker is uppercase and
underlined) is represented by the following:
(SEQ ID NO: 62)
qvevvtqderkalhttaslrcslktsqeplivtwqkkkavspenmvtyskthgvviqpaykdrinvtelglwnssitfwnttledegcy
mclfntfgsqkvsgtacltlyvqpivhlhynyfedhlnitcsatarpapaiswkgtgtgiensteshfhsngttsvtsilrvkdpktqvgke
vicqvlylgnvidykqsldkgGGGSGGGSCSQPLDVILLLDGSSSFPASYFDEMKSFAKAFISKANI
GPRLTQVSVLQYGSITTIDVPWNVVPEKAHLLSLVDVMQREGGPSQIGDALGFAVRYLT
SEMHGARPGASKAVVILVTDVSVDSVDAAADAARSNRVTVFPIGIGDRYDAAQLRILAG
PAGDSNVVKLQRIEDLPTMVTLGNSFLHKLCSGFVRI.
Human CD200 (lower case)-CBD fusion protein (upper case) (linker is uppercase and
underlined) is represented by the following:
(SEQ ID NO: 63)
qvqvvtqdereqlytpaslkcslqnaqealivtwqkkkavspenmvtfsenhgvviqpaykdkinitqlglqnstitfwnitledegcy
mclfntfgfgkisgtacltvyvqpivslhykfsedhlnitcsatarpapmvfwkvprsgienstvtlshpngttsvtsilhikdpknqvgk
evicqvlhlgtvtdfkqtvnkgGGGSGGGSCSQPLDVILLLDGSSSFPASYFDEMKSFAKAFISKANI
GPRLTQVSVLQYGSITTIDVPWNVVPEKAHLLSLVDVMQREGGPSQIGDALGFAVRYLT
SEMHGARPGASKAVVILVTDVSVDSVDAAADAARSNRVTVFPIGIGDRYDAAQLRILAG
PAGDSNVVKLQRIEDLPTMVTLGNSFLHKLCSGFVRI.
Mouse CD200 (uppercase)-mouse serum albumin (lowercase)-CBD (italic underline uppercase)
fusion protein is represented by the following (linkers are uppercase and underlined):
(SEQ ID NO: 64)
QVEVVTQDERKALHTTASLRCSLKTSQEPLIVTWQKKKAVSPENMVTYSKTHGVVIQP
AYKDRINVTELGLWNSSITFWNTTLEDEGCYMCLFNTFGSQKVSGTACLTLYVQPIVHL
HYNYFEDHLNITCSATARPAPAISWKGTGTGIENSTESHFHSNGTTSVTSILRVKDPKTQ
VGKEVICQVLYLGNVIDYKQSLDKGGGGSGGGSeahkseiahryndlgeqhfkglvliafsqylqkcsydeh
aklvqevtdfaktcvadesaancdkslhtlfgdklcaipnlrenygeladcctkqepernecflqhkddnpslppferpeaeamctsfke
npttfmghylhevarrhpyfyapellyyaeqyneiltqccaeadkescltpkldgvkekalvssvrqrmkcssmqkfgerafkawav
arlsqtfpnadfaeitklatdltkvnkecchgdllecaddraelakymcenqatissklqtccdkpllkkahclsevehdtmpadlpaiaa
dfvedqevcknyaeakdvflgtflyeysrrhpdysvslllrlakkyeatlekccaeanppacygtvlaefqplveepknlvktncdlyek
lgeygfqnailvrytqkapqvstptlveaarnlgrvgtkcctlpedqrlpcvedylsailnrvcllhektpvsehvtkccsgslverrpcfsa
ltvdetyvpkefkaetftfhsdictlpekekqikkqtalaelvkhkpkataeqlktvmddfaqfldtcckaadkdtcfstegpnlvtrckda
laGGGSGGGSCSQPLDVILLLDGSSSFPASYFDEMKSFAKAFISKANIGPRLTQVSVLQYGSITTI
DVPWNVVPEKAHLLSLVDVMQREGGPSQIGDALGFAVRYLTSEMHGARPGASKAVVILVTDV
SVDSVDAAADAARSNRVTVFPIGIGDRYDAAQLRILAGPAGDSNVVKLQRIEDLPTMVTLGNSF
LHKLCSGFVRI.
Human CD200 (uppercase)-human serum albumin (lowercase)-CBD (italic underline uppercase)
fusion protein is represented by the following (linkers are uppercase and underlined):
(SEQ ID NO: 65)
QVQVVTQDEREQLYTPASLKCSLQNAQEALIVTWQKKKAVSPENMVTFSENHGVVIQP
AYKDKINITQLGLQNSTITFWNITLEDEGCYMCLFNTFGFGKISGTACLTVYVQPIVSLHY
KFSEDHLNITCSATARPAPMVFWKVPRSGIENSTVTLSHPNGTTSVTSILHIKDPKNQVG
KEVICQVLHLGTVTDFKQTVNKGGGGSGGGSdahksevahrfkdlgeenfkalvliafaqylqqcpfedhvkl
vnevtefaktcvadesaencdkslhtlfgdklctvatlretygemadccakqepernecflqhkddnpnlprlvrpevdvmctafhdne
etflkkylyeiarrhpyfyapellffakrykaafteccqaadkaacllpkldelrdegkassakqrlkcaslqkfgerafkawavarlsqrfp
kaefaevsklvtdltkvhtecchgdllecaddradlakyicenqdsissklkeccekpllekshciaevendempadlpslaadfveskd
vcknyaeakdvflgmflyeyarrhpdysvvlllrlaktyettlekccaaadphecyakvfdefkplveepqnlikqncelfeqlgeykfq
nallvrytkkvpqvstptlvevsrnlgkvgskcckhpeakrmpcaedylsvvlnqlcvlhektpvsdrvtkccteslvnrrpcfsalevd
etyvpkefnaetftfhadictlsekerqikkqtalvelvkhkpkatkeqlkavmddfaafvekcckaddketcfaeegkklvaasqaalg
lGGGSGGGSCSQPLDVILLLDGSSSFPASYFDEMKSFAKAFISKANIGPRLTQVSVLQYGSITTI
DVPWNVVPEKAHLLSLVDVMQREGGPSQIGDALGFAVRYLTSEMHGARPGASKAVVILVTDV
SVDSVDAAADAARSNRVTVFPIGIGDRYDAAQLRILAGPAGDSNVVKLQRIEDLPTMVTLGNSF
LHKLCSGFVRI.

IV. ECM-affinity peptides

Collagen is an extracellular matrix (ECM)-protein that regulates a variety of cellular biological functions, such as proliferation, differentiation, and adhesion in both normal and tumor tissue (Ricard-Blum, Cold Spring Harb Perspect Biol 3: a004978, 2011). Collagen is the most abundant protein in the mammalian body and exists in almost all tissues in one or more of 28 isoforms (Ricard-Blum, Cold Spring Harb Perspect Biol 3: a004978, 2011). The blood vessel sub-endothelial space is rich in collagen. Because of its insolubility under physiological conditions, collagen barely exists within the blood (Dubois et al., Blood 107:3902-06, 2006; Bergmeier and Hynes, Cold Spring Harb Perspect Biol 4: a005132, 2012). Tumor vasculature is reported to be permeable due to an abnormal structure (Nagy et al., British journal of cancer 100:865, 2009). Thus, with its leaky vasculature, collagen is exposed in the tumor (Liang et al., Journal of controlled release 209:101-109, 2015; Liang et al., Sci Rep 6:18205, 2016; Yasunaga et al., Bioconjugate Chemistry 22:1776-83, 2011; Xu et al. The Journal of cell biology 154:1069-80, 2001; Swartz and Lund, Nat Rev Cancer 12:210-19). Also, tumor tissue contains increased amounts of collagen compared to normal tissues (Zhou et al. J Cancer 8:1466-76, 2017; Provenzano et al. BMC Med 6:11, 2008).

von Willebrand factor (vWF) is a blood coagulation factor and binds to both type I and type III collagen, and the adhesion receptor GPIb on blood platelets (Lenting et al., Journal of thrombosis and haemostasis: JTH 10:2428-37, 2012; Shahidi Advances in experimental medicine and biology 906:285-306, 2017). When injured, collagen beneath endothelial cells is exposed to blood plasma, and vWF-collagen binding initiates the thrombosis cascade (Shahidi Advances in experimental medicine and biology 906:285-306, 2017; Wu et al. Blood 99:3623-28, 2002). The vWF A domain has the highest affinity against collagen among reported non-bacterial origin proteins/peptides (Addi et al., Tissue Engineering Part B: Reviews, 2016). Particularly within the A domain, the A3 domain of vWF has been reported as a collagen binding domain (CBD) (Ribba et al. Thrombosis and Haemostasis 86:848-54, 2001). As described above, the inventors contemplated that a fusion protein with the vWF A3 CBD may achieve targeted cytokine immunotherapy even when injected systemically due to exposure of collagen via the leaky tumor vasculature.

In some embodiments, the ECM-affinity peptide comprises a collagen binding domain from decorin. In some embodiments, the ECM-affinity peptide comprises a decorin peptide such as LRELHLNNNC (SEQ ID NO:1), which is derived from bovine or LRELHLDNNC (SEQ ID NO: 2), which is derived from human.

In some embodiments, the ECM-peptide comprises a peptide fragment from human decorin, which is represented by the following amino acid sequence:

(SEQ ID NO: 3)
CGPFQQRGLFDFMLEDEASGIGPEVPDDRDFEPSLGPVCPFRCQCHLRVV
QCSDLGLDKVPKDLPPDTTLLDLQNNKITEIKDGDFKNLKNLHALILVNN
KISKVSPGAFTPLVKLERLYLSKNQLKELPEKMPKTLQELRAHENEITKV
RKVTFNGLNQMIVIELGTNPLKSSGIENGAFQGMKKLSYIRIADTNITSI
PQGLPPSLTELHLDGNKISRVDAASLKGLNNLAKLGLSFNSISAVDNGSL
ANTPHLRELHLDNNKLTRVPGGLAEHKYIQVVYLHNNNISVVGSSDFCPP
GHNTKKASYSGVSLFSNPVQYWEIQPSTFRCVYVRS AIQLGNYK.

In some embodiments, the ECM-peptide comprises a peptide fragment from vWF. In some embodiments, the ECM-peptide comprises vWF A1 derived from human sequence, residues 1237-1458 (474-695 of mature VWF) or a fragment thereof, which is represented by the amino acid sequence

(SEQ ID NO: 4)
CQEPGGLVVPPTDAPVSPTTLYVEDISEPPLHDFYCSRLLDLVFLLDGSS
RLSEAEFEVLKAFVVDMMERLRISQKWVRVAVVEYHDGSHAYIGLKDRKR
PSELRRIASQVKYAGSQVASTSEVLKYTLFQIFSKIDRPEASRITLLLMA
SQEPQRMSRNFVRYVQGLKKKKVIVIPVGI GPHANLKQIRLIEKQAPEN
K AFVLSSVDELEQQRDEIVSYLC.

In some embodiments, the ECM-peptide comprises all or a fragment of vWF A3, which is represented by the following amino acid sequences:

(SEQ ID NO: 5)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRICTG
and
(SEQ ID NO: 47)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRI.

In some embodiments, the ECM-peptide comprises all or a fragment of vWF A3, which is represented by the following amino acid sequences:

(SEQ ID NO: 52)
CSQPLDVVLLLDGSSSLPESSFDKMKSFAKAFISKANIGPHLTQVSVIQY
GSINTIDVPWNVVQEKAHLQSLVDLMQQEGGPSQIGDALAFAVRYVTSQI
HGARPGASKAVVIIIMDTSLDPVDTAADAARSNRVAVFPVGVGDRYDEAQ
LRILAGPGASSNVVKLQQVEDLSTMATLGNSFFHKLCSGFSGV.

In some embodiments, the ECM-affinity peptide is a peptide from von Willebrand factor (vWF). The sequence of human VWF comprises the following:

(SEQ ID NO: 6)
MIPARFAGVLLALALILPGTLCAEGTRGRSSTARCSLFGSDFVNTFDGSM
YSFAGYCSYLLAGGCQKRSFSIIGDFQNGKRVSLSVYLGEFFDIHLFVNG
TVTQGDQRVSMPYASKGLYLETEAGYYKLSGEAYGFVARIDGSGNFQVLL
SDRYFNKTCGLCGNFNIFAEDDFMTQEGTLTSDPYDFANSWALSSGEQWC
ERASPPSSSCNISSGEMQKGLWEQCQLLKSTSVFARCHPLVDPEPFVALC
EKTLCECAGGLECACPALLEYARTCAQEGMVLYGWTDHSACSPVCPAGME
YRQCVSPCARTCQSLHINEMCQERCVDGCSCPEGQLLDEGLCVESTECPC
VHSGKRYPPGTSLSRDCNTCICRNSQWICSNEECPGECLVTGQSHFKSFD
NRYFTFSGICQYLLARDCQDHSFSIVIETVQCADDRDAVCTRSVTVRLPG
LHNSLVKLKHGAGVAMDGQDVQLPLLKGDLRIQHTVTASVRLSYGEDLQM
DWDGRGRLLVKLSPVYAGKTCGLCGNYNGNQGDDFLTPSGLAEPRVEDFG
NAWKLHGDCQDLQKQHSDPCALNPRMTRFSEEACAVLTSPTFEACHRAVS
PLPYLRNCRYDVCSCSDGRECLCGALASYAAACAGRGVRVAWREPGRCEL
NCPKGQVYLQCGTPCNLTCRSLSYPDEECNEACLEGCFCPPGLYMDERGD
CVPKAQCPCYYDGEIFQPEDIFSDHHTMCYCEDGFMHCTMSGVPGSLLPD
AVLSSPLSHRSKRSLSCRPPMVKLVCPADNLRAEGLECTKTCQNYDLECM
SMGCVSGCLCPPGMVRHENRCVALERCPCFHQGKEYAPGETVKIGCNTCV
CRDRKWNCTDHVCDATCSTIGMAHYLTFDGLKYLFPGECQYVLVQDYCGS
NPGTFRILVGNKGCSHPSVKCKKRVTILVEGGEIELFDGEVNVKRPMKDE
THFEVVESGRYIILLLGKALSVVWDRHLSISVVLKQTYQEKVCGLCGNED
GIQNNDLTSSNLQVEEDPVDFGNSWKVSSQCADTRKVPLDSSPATCHNNI
MKQTMVDSSCRILTSDVFQDCNKLVDPEPYLDVCIYDTCSCESIGDCACF
CDTIAAYAHVCAQHGKVVTWRTATLCPQSCEERNLRENGYECEWRYNSCA
PACQVTCQHPEPLACPVQCVEGCHAHCPPGKILDELLQTCVDPEDCPVCE
VAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFV
VDMMERLRISQKWVRVAVVEYHDGSHAYIGLKDRKRPSELRRIASQVKYA
GSQVASTSEVLKYTLFQIFSKIDRPEASRITLLLMASQEPQRMSRNFVRY
VQGLKKKKVIVIPVGIGPHANLKQIRLIEKQAPENKAFVLSSVDELEQQR
DEIVSYLCDLAPEAPPPTLPPDMAQVTVGPGLLGVSTLGPKRNSMVLDVA
FVLEGSDKIGEADENRSKEEMEEVIQRMDVGQDSIHVTVLQYSYMVTVEY
PFSEAQSKGDILQRVREIRYQGGNRTNTGLALRYLSDHSFLVSQGDREQA
PNLVYMVTGNPASDEIKRLPGDIQVVPIGVGPNANVQELERIGWPNAPIL
IQDFETLPREAPDLVLQRCCSGEGLQIPTLSPAPDCSQPLDVILLLDGSS
SFPASYFDEMKSFAKAFISKANIGPRLTQVSVLQYGSITTIDVPWNVVPE
KAHLLSLVDVMQREGGPSQIGDALGFAVRYLTSEMHGARPGASKAVVILV
TDVSVDSVDAAADAARSNRVTVFPIGIGDRYDAAQLRILAGPAGDSNVVK
LQRIEDLPTMVTLGNSFLHKLCSGFVRICMDEDGNEKRPGDVWTLPDQCH
TVTCQPDGQTLLKSHRVNCDRGLRPSCPNSQSPVKVEETCGCRWTCPCVC
TGSSTRHIVTFDGQNFKLTGSCSYVLFQNKEQDLEVILHNGACSPGARQG
CMKSIEVKHSALSVELHSDMEVTVNGRLVSVPYVGGNMEVNVYGAIMHEV
RFNHLGHIFTFTPQNNEFQLQLSPKTFASKTYGLCGICDENGANDFMLRD
GTVTTDWKTLVQEWTVQRPGQTCQPILEEQCLVPDSSHCQVLLLPLFAEC
HKVLAPATFYAICQQDSCHQEQVCEVIASYAHLCRTNGVCVDWRTPDFCA
MSCPPSLVYNHCEHGCPRHCDGNVSSCGDHPSEGCFCPPDKVMLEGSCVP
EEACTQCIGEDGVQHQFLEAWVPDHQPCQICTCLSGRKVNCTTQPCPTAK
APTCGLCEVARLRQNADQCCPEYECVCDPVSCDLPPVPHCERGLQPTLTN
PGECRPNFTCACRKEECKRVSPPSCPPHRLPTLRKTQCCDEYECACNCVN
STVSCPLGYLASTATNDCGCTTTTCLPDKVCVHRSTIYPVGQFWEEGCDV
CTCTDMEDAVMGLRVAQCSQKPCEDSCRSGFTYVLHEGECCGRCLPSACE
VVTGSPRGDSQSSWKSVGSQWASPENPCLINECVRVKEEVFIQQRNVSCP
QLEVPVCPSGFQLSCKTSACCPSCRCERMEACMLNGTVIGPGKTVMIDVC
TTCRCMVQVGVISGFKLECRKTTCNPCPLGYKEENNTGECCGRCLPTACT
IQLRGGQIMTLKRDETLQDGCDTHFCKVNERGEYFWEKRVTGCPPFDEHK
CLAEGGKIMKIPGTCCDTCEEPECNDITARLQYVKVGSCKSEVEVDIHYC
QGKCASKAMYSIDINDVQDQCSCCSPTRTEPMQVALHCTNGSVVYHEVLN
AMECKCSPRKCSK.

In some embodiments, the peptide is from the vWF A3 domain. The vWF A3 domain is derived from the human sequence, residues 1670-1874 (907-1111 of mature vWF) and has the following sequence:

(SEQ ID NO: 7)
CSGEGLQIPTLSPAPDCSQPLDVILLLDGSSSFPASYFDEMKSFAKAFIS
KANIGPRLTQVSVLQYGSITTIDVPWNVVPEKAHLLSLVDVMQREGGPSQ
IGDALGFAVRYLTSEMHGARPGASKAVVILVTDVSVDSVDAAADAARSNR
VTVFPIGIGDRYDAAQLRILAGPAGDSNVVKLQRIEDLPTMVTLGNSFLH
KLCSG.

In some embodiments, the ECM-affinity peptide comprises a peptide from PIGF-2. PIGF-2 has the following sequence:

(SEQ ID NO: 8)
MPVMRLFPCFLQLLAGLALPAVPPQQWALSAGNGSSEVEVVPFQEVWGRS
YCRALERLVDVVSEYPSEVEHMFSPSCVSLLRCTGCCGDENLHCVPVETA
NVTMQLLKIRSGDRPSYVELTFSQHVRCECRPLREKMKPERRRPKGRGKR
RREKQRPTDCHLCGDAVPRR.

Exemplary PIGF-2 ECM affinity peptides include:

(SEQ ID NO: 9)
RRRPKGRGKRRREKQRPTDCHLCGDAVPRR;
(SEQ ID NO: 10)
RRRPKGRGKRRREKQRPTDCHL;
(SEQ ID NO: 11)
RRPKGRGKRRREKQRPTD;
(SEQ ID NO: 12)
RRRPKGRGKRRREKQ;
(SEQ ID NO: 13)
GKRRREKQ;
(SEQ ID NO: 14)
RRRPKGRG;
and
(SEQ ID NO: 15)
RRKTKGKRKRSRNSQTEEPHP.

In some embodiments, the ECM-affinity peptide is a peptide from CXCL-12y. The sequence of CXCL-12Y is the following: CXCL-12y: KPVSLSYRCPCRFFESHVARANVKHLKILNTPNCALQIVARLKNNNRQVCIDPKLKWIQ EYLEKALNKGRREEKVGKKEKIGKKKROKKRKAAQKRKN (SEQ ID NO:16). An exemplary peptide includes all or part of SEQ ID NO:12 and the following peptide:

(SEQ ID NO: 17)
GRREEKVGKKEKIGKKKRQKKRKAAQKRKN.

The ECM-affinity peptide may be a peptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to an ECM or CBD peptide or fragment of the peptides described above.

A linker sequence may be included in the anti-inflammatory agent-peptide construction. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate that antibody and the peptide.

The ECM-affinity peptides of the disclosure may have affinity to one or more components of the extracellular matrix such as fibronectin, collagen, (collagen type I, collagen type III, and/or collagen type IV) tenascin C, fibrinogen, and fibrin. In certain aspects the ECM-affinity peptide has an affinity for collagen. And in other aspects the ECM-affinity peptide does not bind fibronectin.

In some embodiments, the ECM-affinity peptides and/or anti-inflammatory agent of the disclosure is further linked to a serum protein. Serum proteins include, for example, albumin, globulin, and fibrinogen. Globulins include alpha 1 globulins, alpha 2 globulins, beta globulins, and gamma globulins. The albumin may be mouse, human, bovine, or any other homologous albumin protein. In some embodiments, the albumin comprises human serum albumin, which is encoded by the ALB gene, and exemplified by the following amino acid sequence: kwvtfisllflfssaysrgvfrrdahksevahrfkdlgeenfkalvliafaqylqqcpfedhvklvnevtefaktcvadesaencdkslhtlf gdklctvatlretygemadccakqepernecflqhkddnpnlprlvrpevdvmctafhdneetflkkylyeiarrhpyfyapellffakr ykaafteccqaadkaacllpkldelrdegkassakqrlkcaslqkfgerafkawavarlsqrfpkaefaevsklvtdltkvhtecchgdll ecaddradlakyicenqdsissklkeccekpllekshciaevendempadlpslaadfveskdvcknyaeakdvflgmflyeyarrhp dysvvlllrlaktyettlekccaaadphecyakvfdefkplveepqnlikqncelfeqlgeykfqnallvrytkkvpqvstptlvevsrnlg kvgskcckhpeakrmpcaedylsvvlnqlcvlhektpvsdrvtkccteslvnrrpcfsalevdetyvpkefnaetftfhadictlsekerq ikkqtalvelvkhkpkatkeqlkavmddfaafvekcckaddketcfaeegkklvaasqaalgl (SEQ ID NO:45). In some embodiments, serum albumin comprises a polypeptide having the following sequence:

(SEQ ID NO: 60)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL

In some embodiments, the albumin comprises mouse albumin having the following sequence:

(SEQ ID NO: 46)
eahkseiahryndlgeqhfkglvliafsqylqkcsydehaklyqevtdfa
ktcvadesaancdkslhtlfgdklcaipnlrenygeladcctkqeperne
cflqhkddnpslppferpeaeamctsfkenpttfmghylhevarrhpyfy
apellyyaeqyneiltqccaeadkescltpkldgvkekalvssvrqrmkc
ssmqkfgerafkawavarlsqtfpnadfaeitklatdltkvnkecchgdl
lecaddraelakymcenqatissklqtccdkpllkkahclsevehdtmpa
dlpaiaadfvedqevcknyaeakdvflgtflyeysrrhpdysvslllrla
kkyeatlekccaeanppacygtvlaefqplveepknlvktncdlyeklge
ygfqnailvrytqkapqvstptlveaarnlgrvgtkcctlpedqrlpcve
dylsailnrycllhektpvsehytkccsgslverrpcfsaltvdetyvpk
efkaetftfhsdictlpekekqikkqtalaelvkhkpkataeqlktvmdd
faqfldtcckaadkdtcfstegpnlvtrckdala.

Related embodiments comprise a vWF A3 (uppercase) linked through a glycine serine peptide linker (italic uppercase and underlined) with mouse serum albumin (MSA) (lowercase is MSA):

(SEQ ID NO: 50)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRIGGGSGGG
Seahkseiahryndlgeqhfkglvliafsqylqkcsydehaklvqevtdf
aktcvadesaancdkslhtlfgdklcaipnlrenygeladcctkqepern
ecflqhkddnpslppferpeaeamctsfkenpttfmghylhevarrhpyf
yapellyyaeqyneiltqccaeadkescltpkldgvkekalvssvrqrmk
cssmqkfgerafkawavarlsqtfpnadfaeitklatdltkvnkecchgd
llecaddraelakymcenqatissklqtccdkpllkkahclsevehdtmp
adlpaiaadfvedqevcknyaeakdvflgtflyeysrrhpdysyslllrl
akkyeatlekccaeanppacygtvlaefqplfeepknlvktncdlyeklg
eygfqnailvrytqkapqvstptlveaarnlgrvgtkcctlpedqrlpcv
edylsailnrvcllhektpvsehvtkccsgslverrpcfsaltvdetyyp
kefkaetftfhsdictlpekekqikkqtalaelvkhkpkataeqlktvmd
dfaqfldtcckaadkdtcfstegpnlvtrckdala.

Further related embodiments comprise a vWF A3 (uppercase) linked through a glycine serine peptide linker (italic uppercase and underlined) with mouse serum albumin (MSA) (lowercase is MSA): vWF A3 (uppercase) linked through a glycine serine peptide linker (italic uppercase and underlined) with human serum albumin (HSA) (lowercase is HSA):

(SEQ ID NO: 51)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRIGGGSGGG
Skwvtfisllflfssaysrgvfrrdahksevahrfkdlgeenflcalvli
afaqylqqcpfedhvklvnevtefaktcvadesaencdkslhtlfgdklc
tvatlretygemadccakqepernecflqhkddnpnlprlvrpevdvmct
afhdneetflkkylyeiarrhpyfyapellffakrykaafteccqaadka
acllpkldelrdegkassakqrlkcaslqkfgerafkawavarlsqrfpk
aefaevsklvtdltkvhtecchgdllecaddradlakyicenqdsisskl
keccekpllekshciaevendempadlpslaadfveskdvcknyaeakdv
flgmflyeyarrhpdysvvlllrlaktyettlekccaaadphecyakvfd
efkplveepqnlikqncelfeqlgeykfqnallvrytkkvpqvstptlve
vsrnlgkvgskcckhpeakrmpcaedylsvvlnqlcvlhektpvsdrvtk
ccteslvnrrpcfsalevdetyvpkefnaetftfhadictlsekerqikk
qtalvelvkhkpkatkeqlkavmddfaafvekcckaddketcfaeegkkl
vaasqaalgl.

### V_{L}. Proteinaceous Compositions

The polypeptides or polynucleotides of the disclosure, such as the ECM-affinity peptide, serum protein, or cytokine polypeptide, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs: 1-66.

The polypeptides or polynucleotides of the disclosure, such as the ECM-affinity peptide, serum protein, or cytokine polypeptide, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NO:1-66.

In some embodiments, a polypeptide of the disclosure may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOs: 1-66.

In some embodiments, a polypeptide of the disclosure, such as the ECM-affinity peptide, serum protein, or cytokine polypeptide, may comprise at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-66.

In some embodiments, the polypeptide, such as the ECM-affinity peptide, serum protein, or cytokine polypeptide, may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-66 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with any one of SEQ ID NOS: 1-66.

A polypeptide of the disclosure, such as an ECM-affinity peptide, serum protein, or cytokine polypeptide, may be at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any range derivable therein) similar, identical, or homologous with one of SEQ ID NOS: 1-66.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of any of SEQ ID NOS: 1-66 and comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 contiguous nucleotides or amino acids of any of SEQ ID NOS: 1-66.

The polypeptides and nucleic acids of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of one of SEQ ID NO:1-66. One or more of these substitutions may be specifically excluded from an embodiment.

Peptides, polypeptides, and proteins of the disclosure, such as the ECM-affinity peptide, serum protein, or cytokine polypeptide, having at least, having at least, or having 70, 75, 80, 85, 90, 91, 92, 93, 94, 95, 96, 97, 98, 99, 100% identity to any one of SEQ ID NO: 1-66 includes a fragment or segment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any range derivable therein) and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205 (or any range derivable therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. One or more of these substitutions may be specifically excluded from an embodiment.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N-or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### VI. Nucleic Acids

In certain embodiments, the current disclosure concerns recombinant polynucleotides encoding the proteins, polypeptides, and peptides of the invention, such as ECM-affinity peptide operatively linked to anti-inflammatory agents and/or other molecules. Therefore, certain embodiments relate to nucleotides encoding for an ECM-affinity polypeptide and/or an ECM-affinity polypeptide or fragment thereof fused to an anti-inflammatory agent or fragment thereof.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs (or any range derivable therein), including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular embodiments, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other embodiments, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain embodiments of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENER's COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### VII. Combination Therapy

The compositions and related methods of the present disclosure, particularly administration of and ECM-affinity peptide operatively linked to an anti-inflammatory agent and/or other molecules may also be used in combination with the administration of additional therapies such as the additional therapeutics described herein or in combination with other traditional therapeutics known in the art for the treatment of autoimmune or inflammatory conditions.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Various combination regimens of the therapeutic agents and treatments may be employed. Non-limiting examples of such combinations are shown below, wherein a therapeutic agent such as a composition disclosed herein is “A” and a second agent, such as an additional agent or therapy described herein or known in the art is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

In some embodiments, more than one course of therapy may be employed. It is contemplated that multiple courses may be implemented.

VIII. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations.

The autoimmune condition or inflammatory condition amenable for treatment may include, but not be limited to conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano-or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopeniarpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

### IX. Pharmaceutical Compositions and Methods

In some embodiments, pharmaceutical compositions are administered to a subject. Different aspects involve administering an effective amount of a composition to a subject. In some embodiments, a composition comprising an anti-inflammatory agent may be administered to the subject or patient to treat inflammation and/or autoimmunity. Additionally, such compounds can be administered in combination with an additional treatment.

Compositions can be formulated for parenteral administration, e.g., formulated for injection via the intravenous, transcatheter injection, intraarterial injection, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. Other routes of administration include intratumoral, peri-tumoral, intralymphatic, injection into inflamed tissue, or into lymph nodes. In some embodiments, the administration is systemic.

Other routes of administration are also contemplated. For example, the constructs and agents may be administered in association with a carrier. In some embodiments, the carrier is a nanoparticle or microparticle.

Particles can have a structure of variable dimension and known variously as a microsphere, microparticle, nanoparticle, nanosphere, or liposome. Such particulate formulations can be formed by covalent or non-covalent coupling of the construct to the particle. By “particle,” “microparticle,” “bead,” “microsphere,” and grammatical equivalents herein is meant small discrete particles that are administrable to a subject. In certain embodiments, the particles are substantially spherical in shape. The term “substantially spherical,” as used herein, means that the shape of the particles does not deviate from a sphere by more than about 10%. The particles typically consist of a substantially spherical core and optionally one or more layers. The core may vary in size and composition. In addition to the core, the particle may have one or more layers to provide functionalities appropriate for the applications of interest. The thicknesses of layers, if present, may vary depending on the needs of the specific applications. For example, layers may impart useful optical properties.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or ug/kg or micrograms/kg/minute or mg/kg/min or micrograms/kg/hour or mg/kg/hour, or μM or mM of an agent discussed herein. Any range derivable therein is contemplated.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### X. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Engineering of Collagen-Binding Modification Enhanced the Efficacy of Anti-Inflammatory Agents

### A. Results

### 1. CBP-Conjugation Provided Collagen Affinity to Anti-TNFα Antibody (αTNF)

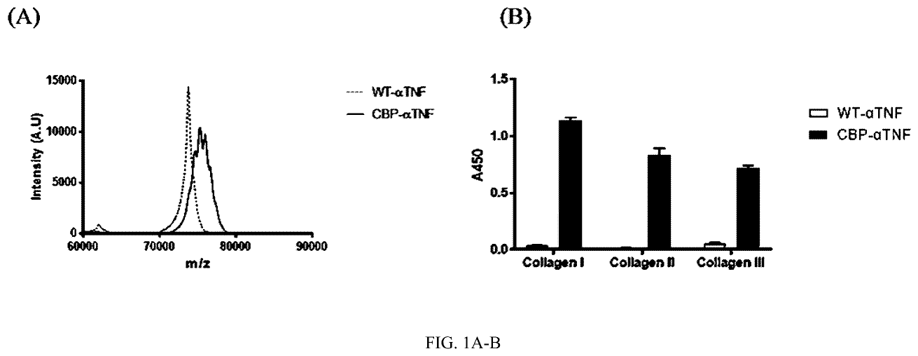

After mixing anti-TNFα antibody (αTNF) with sulfosuccinimidyl-4-(N-maleimidomethyl) cyclohexane-1-carboxylate (sulfo-SMCC), the CBP was cross-linked covalently to the antibody. Up to 5 CBPs were bound to the antibody as calculated by matrix-assisted laser desorption/ionization-time-of-flight (MALDI-TOF) mass spectrometry (FIG. 1A). To examine the capacities of CBP-conjugated αTNF (CBP-αTNF) to bind collagens, the binding activities of CBP-αTNF and unmodified αTNF (WT-αTNF) against types I, II, and III collagen were determined by ELISA. CBP-αTNF bound to all tested types of collagen, whereas WT-αTNF binding signal to collagen was at undetectable level (FIG. 1B).

### 2. CBP-Conjugation Enabled αTNF to Localize in the Inflamed Paw of the Arthritis Model

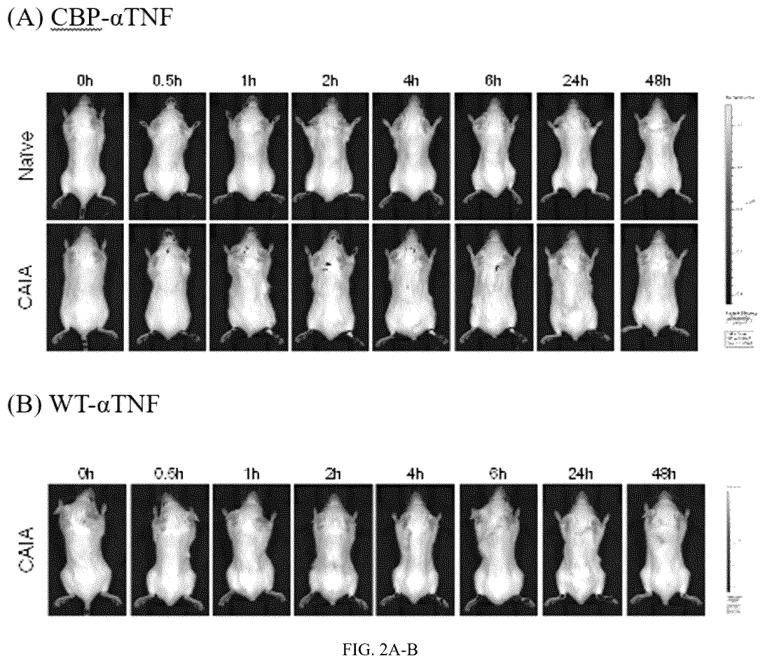

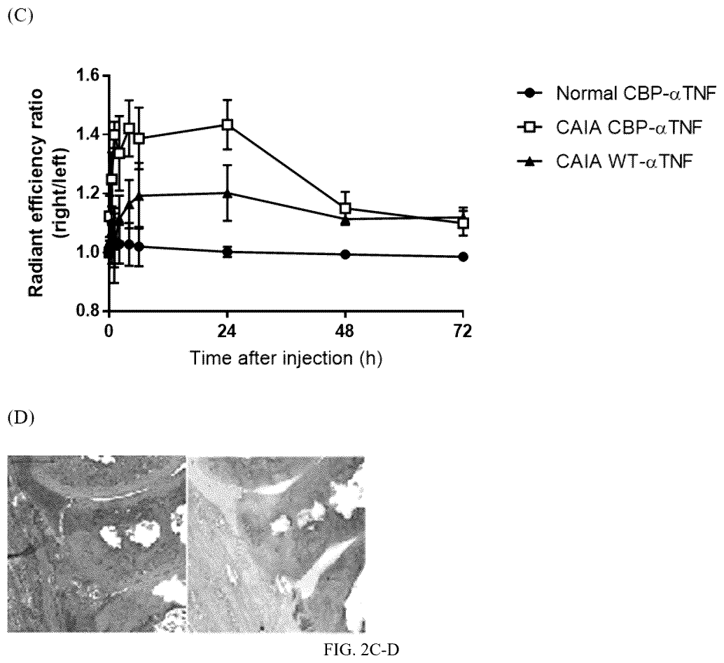

Localization of CBP-αTNF in the inflamed paw of the collagen antibody-induced arthritis (CAIA) model through binding to endogenous collagen was determined by in vivo bio-distribution analysis. Arthritis was induced selectively in right hind paw by passive immunization of anti-collagen antibodies, followed by subcutaneous injection of LPS at right hind footpad. The local LPS injection induced severe arthritis at the right hind paw compared with the other paws. On the following day of LPS injection, fluorescence labeled CBP-αTNF and WT-αTNF was intravenously injected into the CAIA and naïve mice. Fluorescence level in whole body was measured before the antibody injection and at 0.5, 1, 2, 4, 6, 24, and 48 hours after the injection. The fluorescence level in right hind arthritic paw of the CAIA mice injected with CBP-αTNF and WT-αTNF increased immediately after the injection, whereas that of naïve mice was almost the same in both arthritic and non-arthritic paws (FIGS. 2A and 2B). The ratio of the level in arthritic paw to non-arthritic paw was higher in mice injected with CBP-αTNF than WT-αTNF (FIG. 2C). The injected CBP-αTNF was detected in the synovium and pannus, the principal inflamed area of arthritis by the immunohistochemistry (FIG. 2D). These data indicate that CBD-αTNF preferentially localizes to the inflamed tissue (i.e. arthritic paw) after systemic injection, more than its unmodified form.

### 3. CBP-Conjugation Enhanced the Efficacy of αTNF in the Arthritis Model

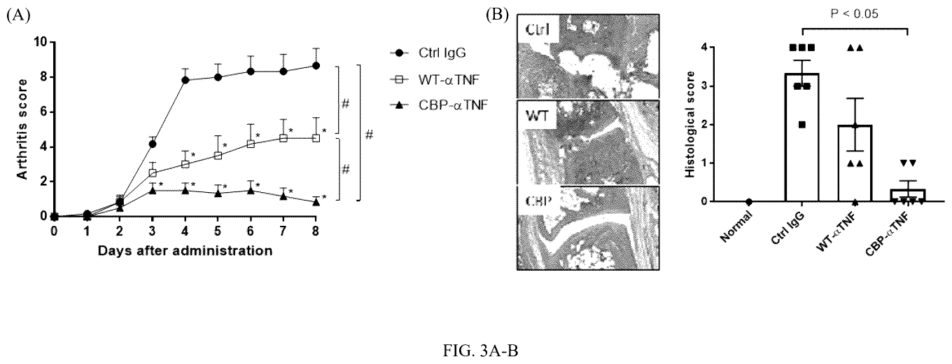

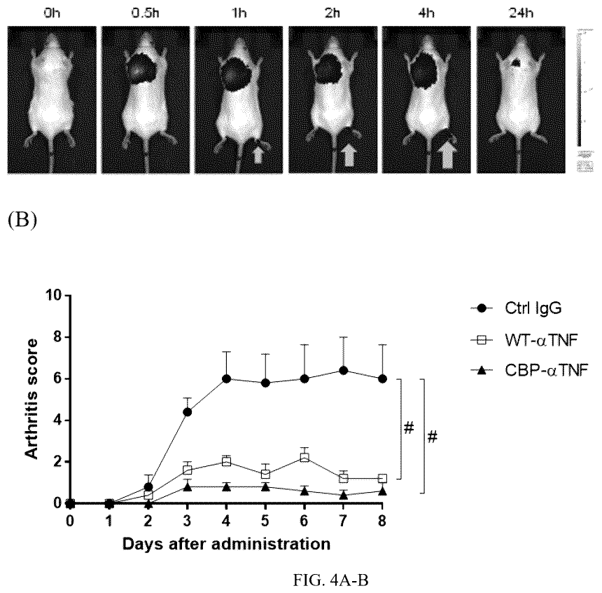

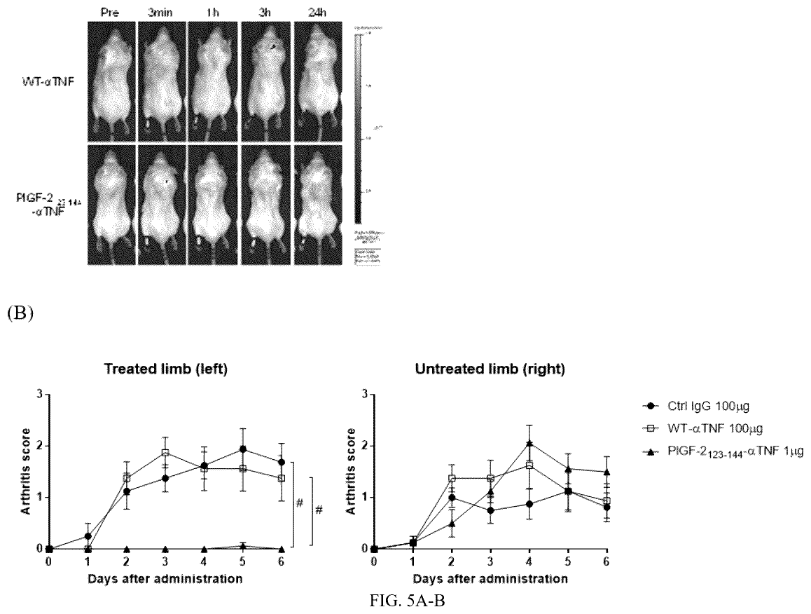

The inventors next examined the anti-inflammatory efficacy of CBP-αTNF in the CAIA model. Arthritis was induced in all paws by passive immunization of anti-collagen antibodies, followed by intraperitoneal injection of LPS. On the day of LPS injection, control IgG, WT-αTNF, or CBP-αTNF was intravenously injected. Arthritis score was increased in control mice and the score was reduced by WT-αTNF and CBP-αTNF (FIG. 3A). The score reduction in CBP-αTNF-treated mice was significantly greater than WT-αTNF-treated mice. The histological observation revealed that the joint destruction was significantly suppressed by CBP-αTNF (FIG. 3B). The inventors further investigated whether CBP-αTNF could show the efficacy by injection via subcutaneous route. Similar tendency in the accumulation and the inhibitory efficacy was observed even in subcutaneous injection of CBP-αTNF (FIG. 4). These data indicate that CBP modification of αTNF provides superior anti-inflammatory efficacy to its unmodified form.

### 4. Local Injection of ECM-Binding αTNF had a Robust Effect on Arthritis Development

To assess the therapeutic potency of localized therapy, the inventors conjugated αTNF with promiscuous ECM-binding peptide derived from placenta growth factor 2 (specifically, PIGF-2₁₂₃₋₁₄₄) in the same manner as CBP conjugation described previously (32, 33). PIGF-2123-144 peptide binds to multiple ECM proteins with high affinity, thus it retains at injection site. PIGF-2₁₂₃₋₁₄₄-conjugated αTNF (PIGF-2₁₂₃₋₁₄₄-αTNF) injected at left hind footpad of CAIA mice retained at the injection site, whereas the signal from WT-αTNF rapidly reduced after the injection (FIG. 5A). To compare the efficacy, control IgG, WT-αTNF, or PIGF-2₁₂₃₋₁₄₄-αTNF was injected subcutaneously at left hind footpad of CAIA mice. Arthritis score was increased in both right and left hind limbs in control IgG-treated mice. WT-αTNF did not suppress the score in this treatment regimen. PIGF-2₁₂₃₋₁₄₄-αTNF, however, suppressed arthritis development almost completely in the treated paw (left) even at 100-times lower dose than WT-αTNF. Interestingly, PIGF-2₁₂₃₋₁₄₄-αTNF did not suppress the arthritis development in the untreated paw (right), indicating its localized efficacy (FIG. 5B). These data suggest that accumulation of drug in inflammatory site is crucial to suppress inflammation and maximize the efficacy of anti-inflammatory drugs.

5. CBD Protein Derived from the vWF A3 Domain can Target Inflamed Spinal Cord in the Experimental Autoimmune Encephalomyelitis (EAE) Model

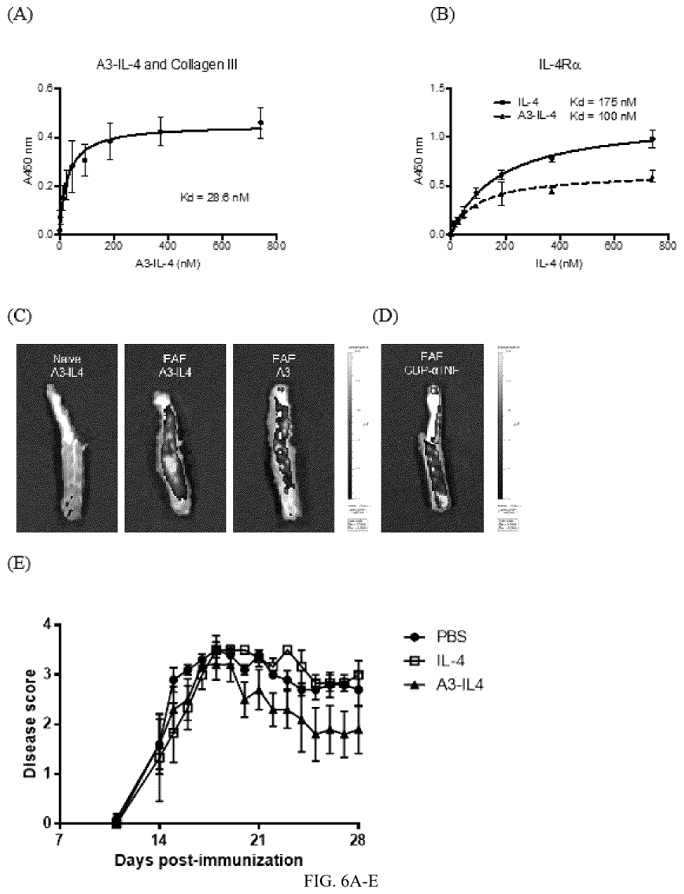

IL-4 is a cytokine that induces differentiation of naïve helper T cells (Th0) to Th2 cells. Application of IL-4 to the central nerve system by intrathecal or intranasal administration has been reported to ameliorate clinical signs and axonal morphology in experimental autoimmune encephalomyelitis (EAE) model, a mouse model for multiple sclerosis. Thus, to achieve EAE targeting of IL-4 from intravenously injection, which is clinically relevant injection route, the inventors synthesized vWF A3 domain-fused IL-4 (A3-IL4) protein. A3-IL4 bound to collagen III with a dissociation constant (Kd) of 28.6 nM (FIG. 6A). vWF A3 domain-fusion to IL-4 did not abolish the binding affinity against its receptor, IL-4Ra (FIG. 6B). Next, localization of the vWF A3 domain protein in the inflamed spinal cord of EAE model through binding to endogenous collagen was determined by fluorescence imaging. On day 14 post-immunization when the target tissues were inflamed, fluorescence-labeled A3 or A3-IL4 was intravenously injected into the naïve and EAE mice. A3 and A3-IL4 were detected in spinal cord of EAE mice, but not in that of a naïve mouse (FIG. 6C). The inventors next examined the therapeutic effect of A3-IL4 on EAE symptoms. From day 14 post-immunization when the first EAE symptoms appeared, PBS, normal form IL-4, and A3-IL4 were intravenously injected every other day. A3-IL4 reduced the mean disease score whereas normal IL-4 did not show any therapeutic effect (FIG. 6E).

### 6. A3 Protein and CBP-Conjugate can Also Target Inflamed Tissues of Other Inflammatory Disease Models

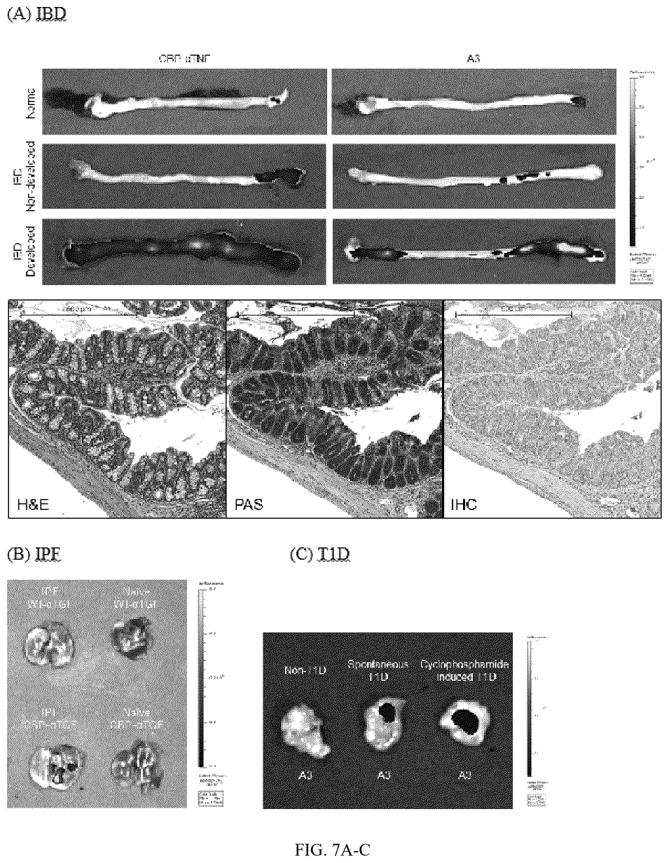

To explore whether the collagen-binding engineering has versatile application for inflammatory diseases, localization of the A3 protein and CBP-conjugated antibody was determined in the inflamed tissues of the spontaneous inflammatory bowel disease (IBD), bleomycin-induced idiopathic pulmonary fibrosis (IPF), and type I diabetes (TID) models by fluorescence imaging. When the target tissue was inflamed, fluorescence-labeled A3, CBP-αTNF, or CBP-conjugated anti-TGF-β antibody (CBP-αTGF) was intravenously injected to EAE, IBD, IPF, and T1D models, and fluorescent level in the target tissues was determined. A3 was detected in the colon of IBD-developed mice, and pancreas of spontaneous TID and cyclophosphamide-induced TID mice, but not in that of healthy mice (FIGS. 7A and 7C). Similarly, CBP-αTNF was detected in the spinal cord of EAE model, and the colon of IBD-developed mice, but not in that of healthy mice (FIGS. 6D and 7A). Histopathological analysis revealed that CBP-αTNF was localized in colonic lamina propria of the IBD model, where there were infiltrating cells (FIG. 7A). Also, CBP-αTGF was detected in the lung of IPF model, whereas unmodified antibody did not (FIG. 7B). These results suggest that installing collagen affinity to antibody and cytokine enables them to target inflamed tissues.

### B. Discussion

In this study, it was demonstrated that installing collagen-binding affinity to anti-inflammatory antibody enhances its retention in the inflamed tissue and its therapeutic potency. Intravenously and subcutaneously injected CBP-αTNF was accumulated in the inflamed paw of CAIA model. Also, CBP conjugation to antibody enabled it to be detected in multiple inflammatory sites of EAE, IBD, and IPF models. This suggests that collagen affinity can target inflamed sites and is widely applicable to various inflammatory diseases. This is because collagen is universally and abundantly present around the vasculature, but is exposed to the blood stream only when hyper permeability of vasculature occurs in inflammatory tissue. Thus, collagen affinity as a drug delivery method is neither tissue, molecular expression, nor disease specific approach, rather general inflammation specific approach. More importantly, CBP-αTNF reduced arthritis score more effectively than unmodified αTNF in CAIA model. αTNF for inflammatory diseases such as RA and IBD does not show complete response in most patients and can have significant side effects (6-10). Therefore, CBP-αTNF holds translational potential to achieve advanced therapy for inflammation and autoimmune diseases.

To assess whether other collagen-binding protein can also target the site of inflammation after systemic injection, the inventors used the vWF A3 domain recombinant protein. A3-IL4 accumulated in the spinal cord and reduced disease scores in EAE model, whereas normal form of IL-4 did not. Targeting neuronal IL-4 signaling is expected to be a new therapeutic strategy to halt disability progression in multiple sclerosis (34). Although intrathecal or intranasal route is proposed for the IL-4 treatment to pass through blood brain barrier, this Example demonstrates that providing collagen-binding ability to IL-4 enabled to reduce clinical signs in EAE model even via intravenous route. It also shows A3 protein accumulation in the inflamed tissue of IBD and T1D models. The inventors used CAIA model for autoantibody-induced acute inflammation, EAE model for autoimmune-mediated chronic inflammation, IBD model for spontaneous developed inflammation, IPF model for fibrosis accompanied by inflammation, and TID for spontaneously developed T-cell mediated autoimmune disease, as principal inflammatory models. These data suggest that collagen-binding antibodies and cytokines are able to achieve efficient antibody and cytokine therapy in multiple inflammatory diseases through accumulating in the site of inflammation.

This example shows that local injection of PIGF-2₁₂₃₋₁₄₄-αTNF retained at injection site and showed strong therapeutic efficacy at a low dose. However, PIGF-2₁₂₃₋₁₄₄-αTNF therapy is effective only when locally injected due to its promiscuous ECM affinity. The main translational advantage of collagen binding approach for inflammation-targeted therapy is that it can target inflammatory site from systemic delivery route. For clinical translation of collagen binding anti-inflammatory drugs, an advantage lies in the use of the A3 domain from vWF or the CBP from decorin is that both naturally exists in the human body, limiting the possibility of immune system recognition. Also, the CBP can be conjugated to antibodies with a simple chemical reaction. The advantage of this feature is in simplicity in production, in that it is possible to work with antibodies for which production has already been optimized. The inventors have shown that CBP was able to conjugate both anti-TNFα and anti-TGFB antibodies in this example. The CBP conjugation synthesis reaction for antibodies can be done in only 90 minutes, using chemistry that is analogous to PEGylation of proteins. The same reaction is used in antibody-drug conjugates, such as in the production of trastuzumab emtansine (35, 36). As to A3-IL4, given that cytokines are small molecules and are generally easy to produce, the inventors chose to recombinantly fuse rather than to conjugate the A3 with IL-4. These features may facilitate development of collagen binding-drug therapy to overcome the barriers to clinical translation.

In conclusion, it was found that installing collagen affinity to antibody and cytokine enables them to target inflammatory sites. Moreover, collagen-binding anti-inflammatory drugs showed high therapeutic efficacy compared with their unmodified forms. This simple approach of an engineered collagen-binding drug may hold potential for clinical translation as an inflammation-targeted therapeutic.

### C. Materials and Methods

### 1. Synthesis of CBP-Conjugated Antibodies

Rat anti-mouse TNF-α antibody (clone XT3.11, BioXcell) was incubated with 20 eq. of sulfo-succinimidyl 4-(N-maleimidomethyl) cyclohexane-1-carboxylate (sulfo-SMCC) for 30 min at room temperature. Excess sulfo-SMCC was removed using a Zeba spin desalting column (Thermo Fisher Scientific). 30 eq. of collagen-binding sequence peptide derived from decorion (CBP, LRELHLNNNC) was then added and reacted for 1 hour at room temperature for conjugation to the thiol moiety on the C residue. The peptide had been synthesized with >95% purity by Genscript.

### 2. Production and Purification of Recombinant vWF A3 Domain and A3-Fused IL-4 Proteins

The sequence encoding for the human vWF A3 domain residues Cys1670-Glyl874 (907-1111 of mature vWF), and the human vWF A3 domain and mouse IL-4 fusion protein were synthesized and subcloned into the mammalian expression vector pcDNA3.1 (+) by Genscript. A sequence encoding for 6 His was added at the N-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×10⁶ cells/ml. 2 μg/ml plasmid DNA, 2 μg/ml linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO₂. 6 days after transfection, the cell culture medium was collected by centrifugation and filtered through a 0.22 μm filter. Culture media was loaded into a HisTrap HP 5 ml column (GE Healthcare), using an ÄKTA pure 25 (GE Healthcare). After washing of the column with wash buffer (20 mM imidazole, 20 mM NaH₂PO₄, 0.5 M NaCl, pH 7.4), protein was eluted with a gradient of 500 mM imidazole (in 20 mM NaH₂PO₄, 0.5 M NaCl, pH 7.4). The elusion solution was further purified with size exclusion chromatography using a HiLoad Superdex 200PG column (GE healthcare). All purification steps were carried out at 4° C. The expression of A3 and A3-IL-4 was determined by western blotting using anti-His tag antibody (BioLegend) and the proteins were verified as >90% pure by SDS-PAGE.

### 3. Detection of CBP-αTNF or A3-IL4 Binding to Collagen Proteins

The measurement is performed as described previously (32). 96-well ELISA plates (Greiner Bio One) were coated with types I, II, and III human collagen (10 μg/mL each in PBS, Millipore Sigma) for overnight at 37° C., followed by blocking with 1% BSA in PBS with 0.05% Tween 20 (PBS-T) for 1 hour at room temperature. Then, wells were washed with PBS-T and further incubated with 1 μM CBP-αTNF, 1 μM unmodified αTNF, or 0-740 nM A3-IL4 for 1 hour at room temperature. After 3 washes with PBS-T, antibody was detected by HRP-conjugated antibody against rat IgG, incubated 1 hour at room temperature (Jackson ImmunoResearch). A3-IL4 was detected by specific antibody against mouse IL-4 (R&D Systems). After washes, bound proteins were detected with tetramethylbenzidine substrate by measurement of the absorbance at 450 nm with subtraction of the absorbance at 570 nm.

### 4. Detection of A3-IL4 Binding to its Receptor

The measurement is performed as described previously (32). 96-well ELISA plates (Greiner Bio One) were coated with recombinant mouse IL-4Rα protein (10 μg/mL each in PBS, R&D Systems) for overnight at 37° C., followed by blocking with 1% BSA in PBS with PBS-T for 1 hour at room temperature. Then, wells were washed with PBS-T and further incubated with 0-740 nM A3-IL4 or IL-4 for 1 hour at room temperature. After 3 washes with PBS-T, IL-4 was detected by specific antibody against mouse IL-4 (R&D Systems). After washes, bound proteins were detected with tetramethylbenzidine substrate by measurement of the absorbance at 450 nm with subtraction of the absorbance at 570 nm.

### 5. MALDI-TOF MS

Antibodies were analyzed by MALDI-TOF MS (Bruker Ultraflextreme MALDI TOF/TOF). All spectra were collected with acquisition software Bruker flexControl™ and processed with analysis software Bruker flexAnalysis™ First, a saturated solution of the matrix, a-cyano-4-hydroxycinnamic acid (Sigma-Aldrich), was prepared in 50:50 acetonitrile: 1% TFA in water as a solvent. The analyte in PBS (5 μL, 0.1 mg/mL) and the matrix solution (25 μL) were then mixed, and 1 μL of that mixture was deposited on the MTP 384 ground steel target plate. The drop was allowed to dry in a nitrogen gas flow, which resulted in the formation of uniform sample/matrix co-precipitate. All samples were analyzed using high mass linear positive mode method with 2500 laser shots at the laser intensity of 75%. The measurements were externally calibrated at three points with a mix of carbonic anhydrase, phosphorylase B, and bovine serum albumin.

### 6. In Vivo Bio-Distribution Study

Anti-TNFα antibody (clone XT3.11, BioXcell) and anti-TGF-B antibody (clone 1D11.16.8, BioXcell) were incubated with 8 eq. of SM (PEG) 24 (Thermo Fisher Scientific) for 30 min at room temperature. Excess SM (PEG) 24 was removed using a Zeba spin desalting column (Thermo Fisher Scientific). 30 eq Cy7-labeled CBP ([Cy7] LRELHLNNNC [COOH]) was then added and reacted for 30 min at room temperature for conjugation to the thiol moiety on the C residue. The peptide had been synthesized with >95% purity by Genscript. Unreacted dye was removed by dialylsis against PBS. As to CBP-unconjugated antibodies, αTNF and αTGF were labeled using sulfo-Cy7 NHS ester (Lumiprobe) according to the manufacture's instruction. A3 and A3-IL4 were labeled using DyLight 800 NHS ester (Thermo Fisher Sientific) according to the manufacture's instruction. When the target tissue of the model mice was inflamed, 10 to 100 μg of Cy7 labeled WT-αTNF, WT-αTGF, CBP-αTNF and CBP-αTGF, or DyLight 800 labeled A3 and A3-IL4 were intravenously injected. Mice organs were harvested and imaged with the Xenogen IVIS Imaging System 100 (Xenogen) under the following conditions: f/stop: 2; optical filter excitation 745 nm; excitation 800 nm; exposure time: 5 sec; small binning.

### 7. Mouse Collagen Antibody-Induced Arthritis (CAIA) Model

Arthritis was induced in female Balb/c mice (7 weeks of age) by intraperitoneal injection of anti-collagen antibody cocktail (1.5 mg/mouse, Chondrex) on day-3, followed by intraperitoneal injection of LPS (50 μg/mouse, Chondrex) on day 0. On the day of LPS injection, mice were injected intravenously or subcutaneously at back with control IgG (200 μg/mouse), WT-αTNF (200 μg/mouse), or CBP-αTNF (200 μg/mouse); or injected subcutaneously at left hind footpad with control IgG (100 μg/mouse), WT-αTNF (100 μg/mouse), or PIGF-2₁₂₃₋₁₄₄-αTNF (1 μg/mouse). Joint swelling was scored everyday as described elsewhere (31). On day 8, hind paws were fixed in 10% neutral formalin (Sigma-Aldrich), decalcified in Decalcifer II (Leica), and then provided to histological analysis.

### 8. Experimental Autoimmune Encephalomyelitis (EAE) Model

EAE was induced in female C57BL/6 mice (13 weeks of age) by immunization with an emulsion of MOG₃₅₋₅₅ in complete Freund's adjuvant (200 μg/mouse, Hooke Laboratories) on day 0, followed by administration of pertussis toxin in PBS (100 ng/mouse) on the day of immunization and the following day. On day 14 post-immunization when the EAE symptoms appeared, treatment of recombinant mouse IL-4 (Peprotech) at 1 μg/mouse or A3-IL4 at 1 μg/mouse (equivalent to 0.4 μg/mouse on a molar basis) was started and repeated every other day. Individual mice were scored daily for disease severity on the basis of the following scale: 0, no clinical disease; 0.5, tail weakness; 1, tail paralysis; 2, hindlimb weakness; 3, hindlimb paralysis; 3.5, forelimb weakness; 4, forelimb paralysis; or 5, moribund or death.

### 9. Spontaneous Colitis Model of Inflammatory Bowel Disease (IBD)

IL-10−/-XTLR-4--(DKO) mice were kindly provided by Cathyn Nagler (The University of Chicago). The DKO mice develop colitis spontaneously and exhibit a high incidence of rectal prolapses. At 29 weeks of age when the first sign of rectal prolapse appeared, the mice were used as IBD-developed mice for imaging analysis. As a control of non-developed IBD, DKO mice at 16 weeks of age and genetic background strain (C57BL/6) mice were used.

### 10. Bleomycin-Induced Idiopathic Pulmonary Fibrosis (IPF) Model

Pulmonary fibrosis accompanied by inflammation were induced in female C57BL/6 mice (9 weeks of age) by an intranasal instillation of 100 μg/mouse of bleomycin (Sigma-Aldrich) in saline. On day 7 after the bleomycin administration when the lung was inflamed, the mice were used for imaging analysis.

### 11. NOD Mouse Model of Type I Diabetes (T1D)

Nonobese diabetic (NOD) mice is known as a spontaneous model of T-cell mediated autoimmune insulin-dependent diabetes mellitus (37, 38). Cyclophosphamide can promote the onset of diabetes in NOD mice (39). For imaging analysis, Balb/c mouse was used as a non-diabetic control, naturally developed diabetic NOD mouse, and diabetic mouse accelerated by intraperitoneal injection of cyclophosphamide (Sigma-Aldrich) at 300 mg/kg. Blood glucose level was used as an index of diabetes development.

### 12. Histological Analyses and Immunohistochemistry

Paraffin-embedded joint tissues from CAIA mice and colon from IBD-developed mice were sliced at 5 μm thickness and stained with H&E and/or PAS for pathological analysis. The severity of synovial hyperplasia and bone resorption for the arthritis model was scored by three-grade evaluation (0-2) according to the previously reported criteria with slight modifications as follows: 0, normal to minimal infiltration of pannus in cartilage and subchondral bone of marginal zone; 1, mild to moderate infiltration of marginal zone with minor cortical and medullary bone destruction; 2, severe infiltration associated with total or near total destruction of joint architecture. The scores in both hind paws were summed up for each mouse (score per mouse total, 0-4).

Immunohistostaining was performed according to standard procedures. Briefly, the sections were incubated in 0.3% H₂O₂ for 20 min, blocked with PBS-buffered 1% BSA for 1 hour, and incubated overnight at 4° C. with HRP-conjugated anti-rat IgG (Jackson ImmunoResearch) for 1 hour at room temperature and visualized with diaminobenzidine.

### 13. Statistical Analysis

Statistical analyses were performed using GraphPad Prism software, and P<0.05 was considered statistically significant. Changes in arthritis scores over time were assessed by repeated-measurement two-way ANOVA, and when interactions were considered significant, data were assessed using Dunnett's multiple comparison test at each time point of measurement. To compare the efficacy of CBP-αTNF with WT-αTNF, the data on day 8 were analyzed again via Tukey's multiple comparison test. For histological scores in the CAIA model, Dunnett's multiple comparison test was used to compare the difference between the control IgG-injected group and the αTNF treatment groups.

### D. REFERENCES

The following references and the publications referred to throughout the specification, to the extent that they provide exemplary procedural or other details supplementary to those set forth herein, are specifically incorporated herein by reference.
1. Moreland L W, Schiff M H, Baumgartner S W, Tindall E A, Fleischmann R M, Bulpitt K J, et al. Etanercept therapy in rheumatoid arthritis. A randomized, controlled trial. Ann Intern Med. 1999;130 (6): 478-86.
2. Maini R, St Clair E W, Breedveld F, Furst D, Kalden J, Weisman M, et al. Infliximab (chimeric anti-tumour necrosis factor alpha monoclonal antibody) versus placebo in rheumatoid arthritis patients receiving concomitant methotrexate: a randomised phase III trial. ATTRACT Study Group. Lancet. 1999;354 (9194): 1932-9.
3. Sandborn W J, Hanauer S B, Katz S, Safdi M, Wolf D G, Baerg R D, et al. Etanercept for active Crohn's disease: a randomized, double-blind, placebo-controlled trial. Gastroenterology. 2001;121 (5): 1088-94.
4. Weinblatt M E, Keystone E C, Furst D E, Moreland L W, Weisman M H, Birbara C A, et al. Adalimumab, a fully human anti-tumor necrosis factor alpha monoclonal antibody, for the treatment of rheumatoid arthritis in patients taking concomitant methotrexate: the ARMADA trial. Arthritis Rheum. 2003; 48 (1): 35-45.
5. Jarnerot G, Hertervig E, Friis-Liby I, Blomquist L, Karlen P, Granno C, et al. Infliximab as rescue therapy in severe to moderately severe ulcerative colitis: a randomized, placebo-controlled study. Gastroenterology. 2005;128 (7): 1805-11.
6. Bongartz T, Sutton A J, Sweeting M J, Buchan I, Matteson E L, Montori V. Anti-TNF antibody therapy in rheumatoid arthritis and the risk of serious infections and malignancies: systematic review and meta-analysis of rare harmful effects in randomized controlled trials. JAMA. 2006;295 (19): 2275-85.
7. Dixon W G, Hyrich K L, Watson K D, Lunt M, Galloway J, Ustianowski A, et al. Drug-specific risk of tuberculosis in patients with rheumatoid arthritis treated with anti-TNF therapy: results from the British Society for Rheumatology Biologics Register (BSRBR). Ann Rheum Dis. 2010; 69 (3): 522-8.
8. Bellis E, Scire CA, Carrara G, Adinolfi A, Batticciotto A, Bortoluzzi A, et al. Ultrasound-detected tenosynovitis independently associates with patient-reported flare in patients with rheumatoid arthritis in clinical remission: results from the observational study STARTER of the Italian Society for Rheumatology. Rheumatology (Oxford). 2016; 55 (10): 1826-36.
9. Lisbona M P, Solano A, Ares J, Almirall M, Salman-Monte TC, Maymo J. ACR/EULAR Definitions of Remission Are Associated with Lower Residual Inflammatory Activity Compared with DAS28 Remission on Hand MRI in Rheumatoid Arthritis. J Rheumatol. 2016; 43 (9): 1631-6.
10. Vreju F A, Filippucci E, Gutierrez M, Di Geso L, Ciapetti A, Ciurea M E, et al. Subclinical ultrasound synovitis in a particular joint is associated with ultrasound evidence of bone erosions in that same joint in rheumatoid patients in clinical remission. Clin Exp Rheumatol. 2016; 34 (4): 673-8.
11. Maeda H, Wu J, Sawa T, Matsumura Y, Hori K. Tumor vascular permeability and the EPR effect in macromolecular therapeutics: a review. J Control Release. 2000;65 (1-2): 271-84.
12. Fang J, Nakamura H, Maeda H. The EPR effect: Unique features of tumor blood vessels for drug delivery, factors involved, and limitations and augmentation of the effect. Adv Drug Deliv Rev. 2011; 63 (3): 136-51.
13. Matsumura Y, Maeda H. A new concept for macromolecular therapeutics in cancer chemotherapy: mechanism of tumoritropic accumulation of proteins and the antitumor agent smancs. Cancer Res. 1986;46 (12 Pt 1): 6387-92.
14. Nehoff H, Parayath N N, Domanovitch L, Taurin S, Greish K. Nanomedicine for drug targeting: strategies beyond the enhanced permeability and retention effect. Int J Nanomedicine. 2014; 9:2539-55.
15. Rahman M, Akhter S, Ahmad J, Ahmad M Z, Beg S, Ahmad FJ. Nanomedicine-based drug targeting for psoriasis: potentials and emerging trends in nanoscale pharmacotherapy. Expert Opin Drug Deliv. 2015;12 (4): 635-52.
16. Maeda H. Polymer therapeutics and the EPR effect. J Drug Target. 2017;25 (9-10): 781-5.
17. McDonald D M. Angiogenesis and remodeling of airway vasculature in chronic inflammation. Am J Respir Crit Care Med. 2001;164 (10 Pt 2): S39-45.
18. Ricard-Blum S. The collagen family. Cold Spring Harb Perspect Biol. 2011; 3 (1): a004978.
19. Dubois C, Panicot-Dubois L, Merrill-Skoloff G, Furie B, Furie BC. Glycoprotein VI-dependent and -independent pathways of thrombus formation in vivo. Blood. 2006;107 (10): 3902-6.
20. Bergmeier W, Hynes R O. Extracellular matrix proteins in hemostasis and thrombosis. Cold Spring Harb Perspect Biol. 2012; 4 (2): a005132.
21. Wynn T A. Cellular and molecular mechanisms of fibrosis. J Pathol. 2008;214 (2): 199-210.
22. Addi C, Murschel F, De Crescenzo G. Design and Use of Chimeric Proteins Containing a Collagen-Binding Domain for Wound Healing and Bone Regeneration. Tissue Eng Part B Rev. 2017;23 (2): 163-82.
23. Svensson L, Heinegard D, Oldberg A. Decorin-binding sites for collagen type I are mainly located in leucine-rich repeats 4-5. J Biol Chem. 1995;270 (35): 20712-6.
24. Nareyeck G, Seidler D G, Troyer D, Rauterberg J, Kresse H, Schonherr E. Differential interactions of decorin and decorin mutants with type I and type VI collagens. Eur J Biochem. 2004; 271 (16): 3389-98.
25. Bidanset D J, Guidry C, Rosenberg L C, Choi H U, Timpl R, Hook M. Binding of the proteoglycan decorin to collagen type VI. J Biol Chem. 1992;267 (8): 5250-6. 26. Kalamajski S, Aspberg A, Oldberg A. The decorin sequence SYIRIADTNIT binds collagen type I. J Biol Chem. 2007; 282 (22): 16062-7.
27. Federico S, Pierce B F, Piluso S, Wischke C, Lendlein A, Neffe A T. Design of Decorin-Based Peptides That Bind to Collagen I and their Potential as Adhesion Moieties in Biomaterials. Angew Chem Int Ed Engl. 2015;54 (37): 10980-4.
28. Lenting P J, Casari C, Christophe O D, Denis C V. von Willebrand factor: the old, the new and the unknown. J Thromb Haemost. 2012; 10 (12): 2428-37.
29. Shahidi M. Thrombosis and von Willebrand Factor. Adv Exp Med Biol. 2017; 906:285-306.
30. Wu D, Vanhoorelbeke K, Cauwenberghs N, Meiring M, Depraetere H, Kotze H F, et al. Inhibition of the von Willebrand (VWF)-collagen interaction by an antihuman VWF monoclonal antibody results in abolition of in vivo arterial platelet thrombus formation in baboons. Blood. 2002;99 (10): 3623-8.
31. Ribba A S, Loisel I, Lavergne J M, Juhan-Vague I, Obert B, Cherel G, et al. Ser968Thr mutation within the A3 domain of von Willebrand factor (VWF) in two related patients leads to a defective binding of VWF to collagen. Thromb Haemost. 2001;86 (3): 848-54.
32. Ishihara J, Fukunaga K, Ishihara A, Larsson H M, Potin L, Hosseinchi P, et al. Matrix-binding checkpoint immunotherapies enhance antitumor efficacy and reduce adverse events. Sci Transl Med. 2017;9 (415).
33. Martino M M, Briquez P S, Guc E, Tortelli F, Kilarski W W, Metzger S, et al. Growth factors engineered for super-affinity to the extracellular matrix enhance tissue healing. Science. 2014; 343 (6173): 885-8.
34. Vogelaar C F, Mandal S, Lerch S, Birkner K, Birkenstock J, Buhler U, et al. Fast direct neuronal signaling via the IL-4 receptor as therapeutic target in neuroinflammation. Sci Transl Med. 2018; 10 (430).
35. Lambert J M, Chari R V. Ado-trastuzumab Emtansine (T-DM1): an antibody-drug conjugate (ADC) for HER2-positive breast cancer. J Med Chem. 2014; 57 (16): 6949-64.
36. Peters C, Brown S. Antibody-drug conjugates as novel anti-cancer chemotherapeutics. Biosci Rep. 2015;35 (4): e00225.
37. Castano L, Eisenbarth G S. Type-I diabetes: a chronic autoimmune disease of human, mouse, and rat. Annu Rev Immunol. 1990; 8:647-79.
38. Bach JF. Insulin-dependent diabetes mellitus as an autoimmune disease. Endocr Rev. 1994;15 (4): 516-42.
39. Charlton B, Bacelj A, Slattery R M, Mandel T E. Cyclophosphamide-induced diabetes in NOD/WEHI mice. Evidence for suppression in spontaneous autoimmune diabetes mellitus. Diabetes. 1989;38 (4): 441-7.

### Example 2: Enhanced Lymph Node Trafficking of Engineered IL-10 Suppresses Murine Models of Rheumatoid Arthritis

Rheumatoid arthritis (RA) is a major autoimmune disease. Although clinical trials using interleukin-10 (IL-10) have been performed as a potential treatment of RA, its therapeutic effects have been limited, potentially due to insufficient residence in lymphoid organs, where antigen recognition primarily occurs. Here, the inventors engineered IL-10 as a fusion with serum albumin (SA) without and with fusion of a collagen binding domain (CBD). SA-IL-10 and CBD-SA-IL-10 exhibited longer circulation times than unmodified IL-10 after intravenous injection; moreover, SA fusion led to enhanced lymph node (LN) accumulation compared with unmodified IL-10. Intravenous SA-IL-10 and CBD-SA-IL-10 treatment restored immune cell composition in the paws to a normal status, elevated the frequency of suppressive M2 macrophages, and protected joint morphology. Intravenous SA-IL-10 and CBD-SA-IL-10 showed similar efficacy as treatment with an anti-TNF-α antibody. SA fusion to IL-10 is a simple but effective engineering strategy to achieve LN accumulation and control of RA.

Rheumatoid arthritis (RA) is an autoimmune disease that is currently controlled through treatment with inhibitors of inflammatory pathways. Pathological features of RA are synovitis and joint destruction, which cause severe pain and joint dysfunction (1, 2). Although the causal antigen for RA has not been fully elucidated, collagen recognition by immune cells plays a key role. During progression of RA, autoantigen-specific T cells, especially Th17 cells, are activated and produce inflammatory cytokines including IL-17. Inflammatory cytokines, such as TNF-α and IL-6, in the joint induce activation of macrophages and neutrophils as mediators of the inflammatory response. These inflammatory cells infiltrate the joints and cause various inflammatory responses including activation of osteoclasts that destroy the bones in the joint (3). The current strategy for RA treatment is symptomatic, and considering that many inflammatory cytokines are involved RA progression, various biological therapeutics, such as antibodies or soluble receptors for TNF-α, have been developed and approved for clinical use (4).

As another type of biological therapeutic, administration of anti-inflammatory cytokines has been studied for treatment of RA to induce systemic suppression of inflammation or tolerance. IL-10 is one such anti-inflammatory cytokine (5-7), and various attempts have been performed to explore IL-10-based autoimmune disease therapeutics (6-8). However, the therapeutic effect of

IL-10 in autoimmune disease is still controversial, possibly because of its short circulating half-life and its uncontrolled biodistribution after systemic administration (8).

In the present study, the inventors engineered IL-10 by fusion of serum albumin (SA) to provide prolonged blood circulation and by fusion of a collagen binding domain (CBD) to provide binding affinity to the inflamed site where the permeability of blood vessels is enhanced and extracellular matrix proteins including collagens are exposed to proteins from the blood (16, 17). Thus, the inventors reasoned that CBD-fusion to SA would add inflammatory site targeting to the SA-fused cytokine. By doing this, the inventors sought to explore if enhanced blood circulation and disease site vasculature targeting synergizes to improve IL-10's therapeutic effects on RA. However, the inventors made the observation that SA fusion to IL-10 not only enhanced circulation time but also accumulation in the lymph nodes (LNs). Here, suppression effects of arthritis by engineered IL-10 were evaluated using a passive collagen antibody-induced arthritis (CAIA) model and an active CIA model in the mouse. The inventors found that CBD fusion enhanced accumulation in the inflamed paw and moreover that SA fusion to IL-10 enhanced trafficking of IL-10 into LNs after intravenous injection. SA-fused IL-10 significantly improved the anti-inflammatory effects of IL-10 in the two murine RA models and functioned similarly to TNF-α blockade.

A. Albumin-Fused IL-10 Binds to FcRn and APCs and Accumulates within the LNs

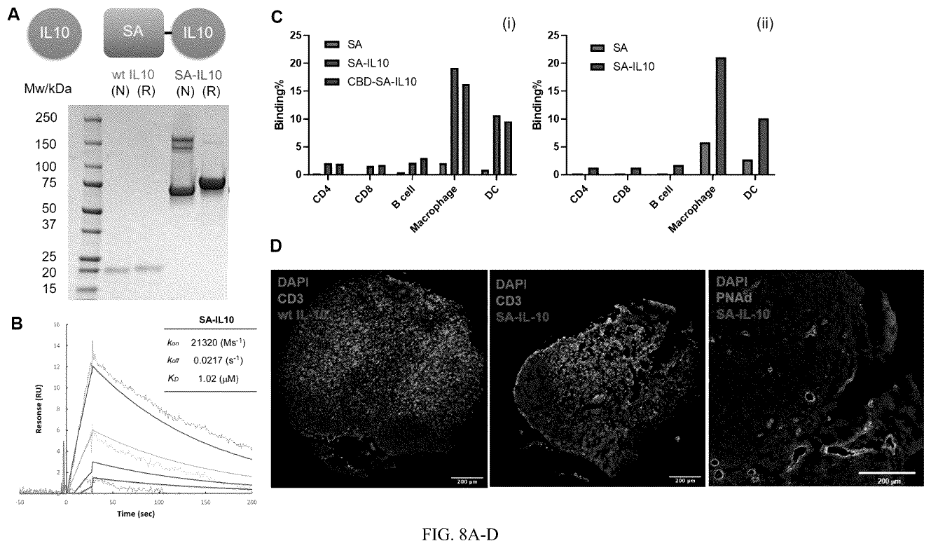

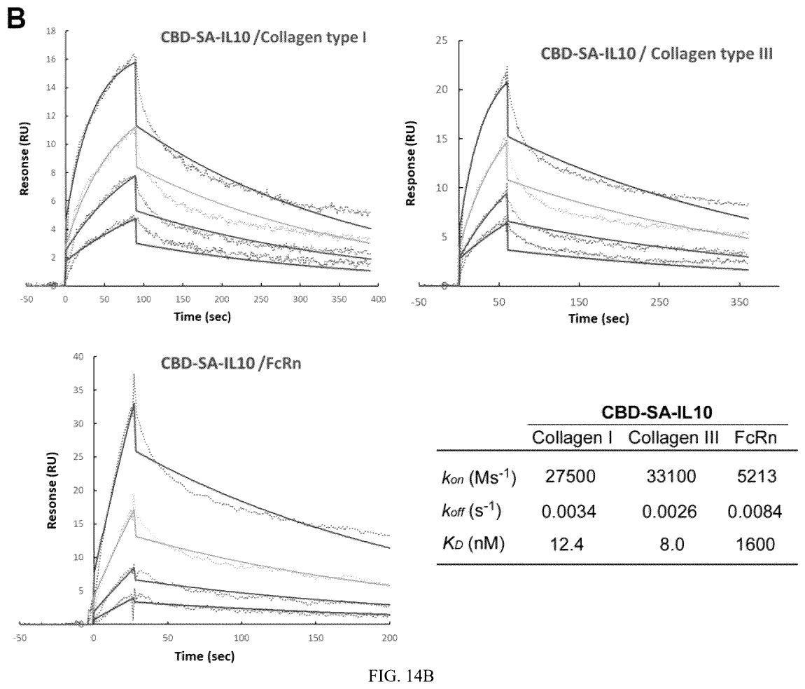

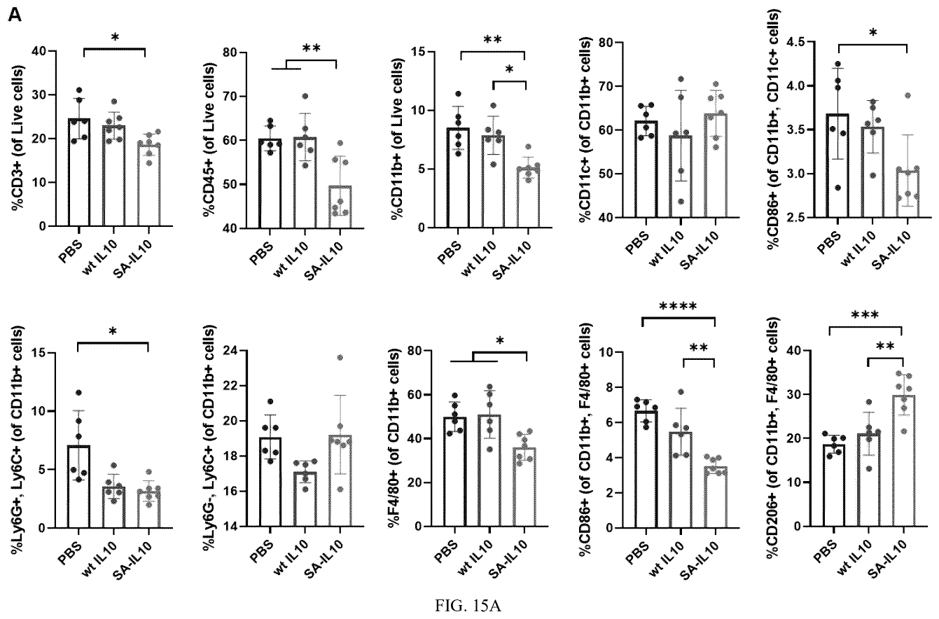

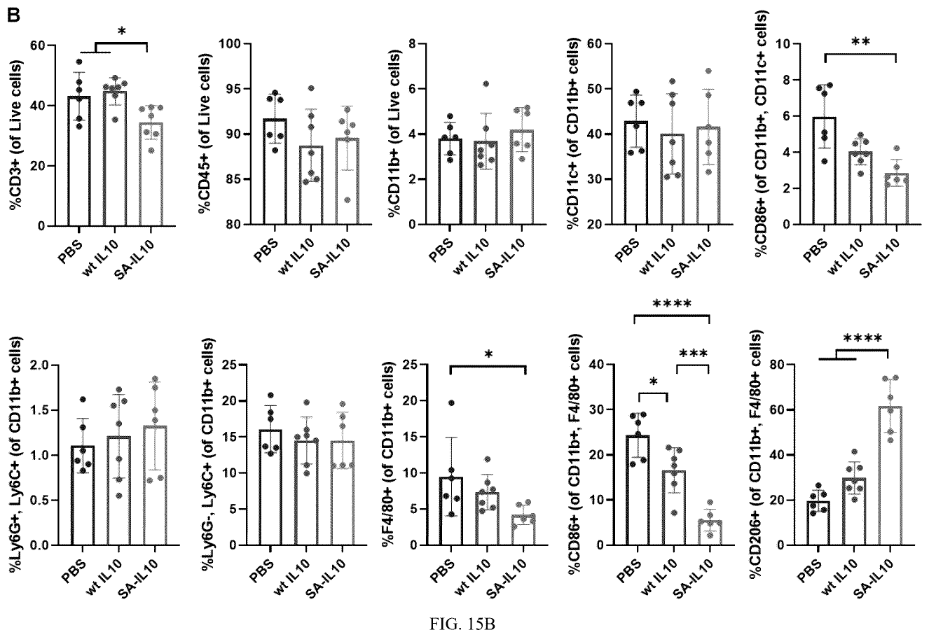

Wild type (wt) mouse IL-10, SA-fused mouse IL-10, and CBD-SA-fused IL-10 were recombinantly expressed, and the molecular weights of the fusion proteins were correspondingly higher than for wt IL-10 as determined by SDS-PAGE; in addition, most of the SA-IL-10 and CBD-SA-IL-10 existed as a monomer under non-reducing conditions (FIGS. 8A and 14A). Surface plasmon resonance (SPR) analysis revealed that SA-IL-10 and CBD-SA-IL-10 bind to neonatal Fc receptor (FcRn) with micromolar order of Ka (FIGS. 8B and 14B). In addition, CBD-SA-IL-10 was shown to bind type I and type III collagens with nanomolar order of Ka (FIG. 14B). The binding ability of these proteins to splenocytes and single cells isolated from the popliteal LN was further evaluated by flow cytometry (FIG. 8C). SA-fused IL-10 exhibited high binding to macrophages and dendritic cells in both splenocytes and LN-derived cells. After intravenous administration of fluorescently-labeled SA-IL-10, significantly higher fluorescence signals were observed within the popliteal LN compared with wt IL-10 (FIG. 8D). Interestingly, higher fluorescence signals were located surrounding high endothelial venules (HEVs), where antigen presenting cells (APCs) reside (18).

### B. Albumin-Fused IL-10 Shows Prolonged Blood Circulation, and CBD Fusion Leads to Accumulation in the Inflamed Paw

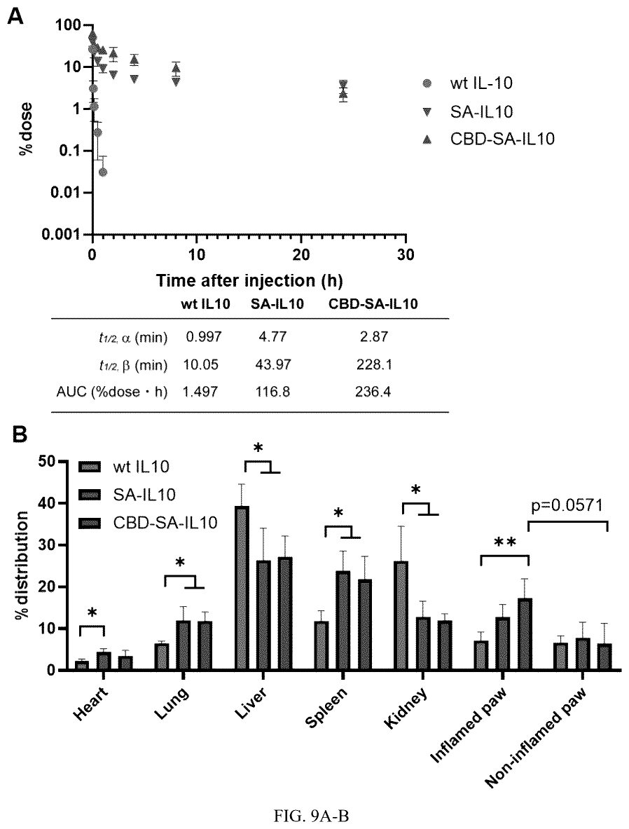

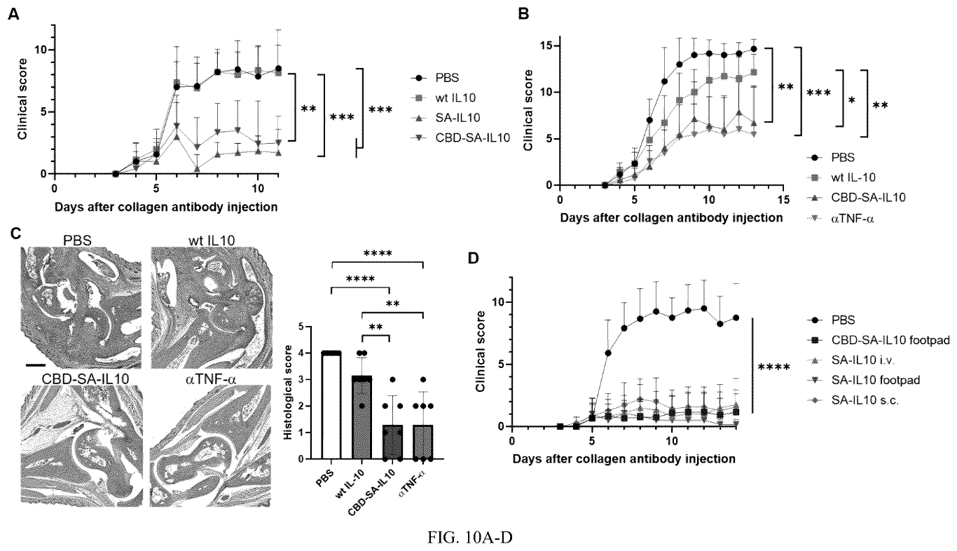

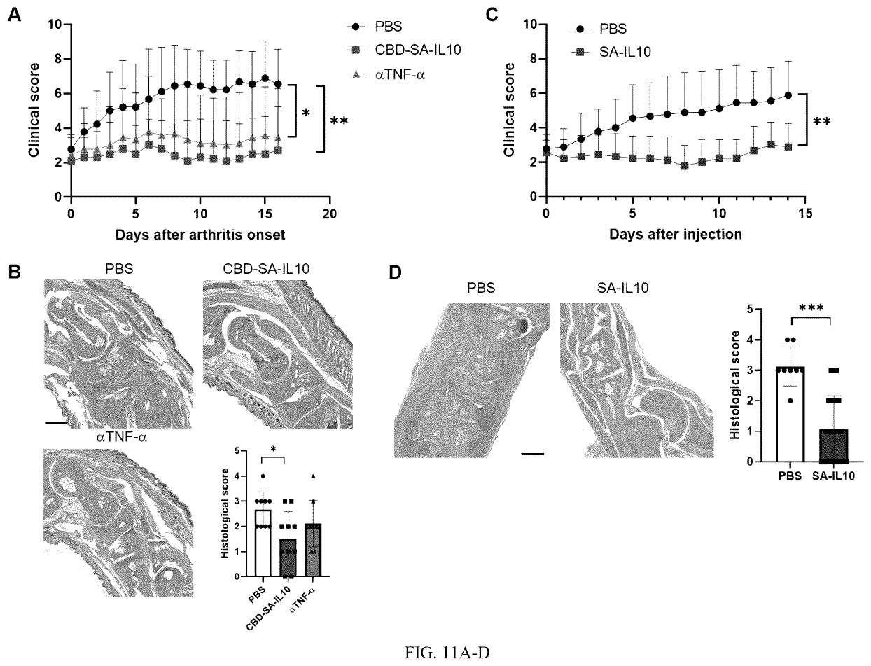

SA is known to demonstrate long circulation via FcRn-mediated recycling on vascular endothelial cells (19, 20). As expected, SA-IL-10 showed significantly prolonged blood circulation compared with wt IL-10; CBD-SA-IL-10 had also comparable circulation as SA-IL-10 (FIG. 9A). FIG. 9B represents the fluorescence signals from major organs of mice intravenously injected with DyLight800-labled proteins. Reflecting their long circulation properties, SA-IL-10 and CBD-SA-IL-10 showed higher signals in the heart, lungs and spleen than that of wt IL-10. In addition, significantly higher signal was detected from the inflamed paw of mice treated with CBD-SA-IL-10 than wt IL-10, whereas the non-inflamed paw exhibited fluorescence that was not significantly different between wt IL-10 and CBD-SA-IL-10, indicating the inflammation targeting capability of CBD-SA-IL-10 through collagen affinity, as reported in previous studies in other contexts (16, 21).

### C. Albumin-Fused IL-10 Suppresses the Development of Arthritis

The therapeutic effects of engineered IL-10 in the passive collagen antibody-induced arthritis (CAIA) model were evaluated (FIG. 10). Intravenous injection of SA-IL-10 or CBD-SA-IL-10 significantly suppressed the development of arthritis, whereas PBS-or wt IL-10-injected mice exhibited severe inflammation in the paws (FIG. 10A). The therapeutic effect of CBD-SA-IL-10 was compared with treatment with anti-TNF-α antibody (αTNF-α), a mouse model of a clinically used antibody drug for treatment of RA, where CBD-SA-IL-10 induced comparable suppression of CAIA as αTNF-α (FIG. 10B). Histological analysis revealed that both CBD-SA-IL-10 and αTNF-α suppressed joint destruction compared with PBS-or wt IL-10 treatment (FIG. 10C). Histological scores also significantly decreased due to treatment with CBD-SA-IL-10 and αTNF-α (FIG. 10C). The effect of the administration route on therapeutic efficacy was also investigated, comparing intravenous, local (footpad), and subcutaneous (at a distant site, mid-back) administration (FIG. 10D). Footpad injection of CBD-SA-IL-10 induced a relatively high suppression effect on CAIA compared with the results of its intravenous injection shown in FIG. 10A and FIG. 10B, suggesting retention of CBD-SA-IL-10 at the inflamed paws via collagen affinity (FIG. 8B). Strikingly, SA-IL-10 showed quite high suppression effects on CAIA by all of the administration routes tested (FIG. 10D).

As a second arthritis model, the active collagen-induced arthritis (CIA) model was used for evaluation of CBD-SA-IL-10 and SA-IL-10 on RA treatment. CBD-SA-IL-10 significantly suppressed the increase of clinical scores compared with PBS-treated CIA mice (FIG. 11A), and its therapeutic effect was comparable with αTNF-α treatment. Histology of the joints and histological scores also revealed suppression of arthritis establishment by CBD-SA-IL-10 (FIG. 11B). Importantly, 5 out of 10 mice treated with CBD-SA-IL-10 showed a score of 1 or less, suggesting a high therapeutic effect of CBD-SA-IL-10. Without the CBD domain, a single injection of SA-IL-10 to CIA mice induced significant suppression of establishment of arthritis compared with PBS (FIG. 11C). Most mice treated with PBS showed severe inflammation at the paws as indicated by histology and the histological score (FIG. 11D). In contrast, SA-IL-10-treated mice exhibited almost identical status in the paw as naïve mice, and most mice showed a histological score of 1 or less. Taken together, these results indicate the highly suppressive effect of inflammation by local or intravenous administration of CBD-SA-IL-10 and by even subcutaneous administration of SA-IL-10.

D. Albumin-Fused IL-10 Reduces Immune Activity after Accumulation within LNs

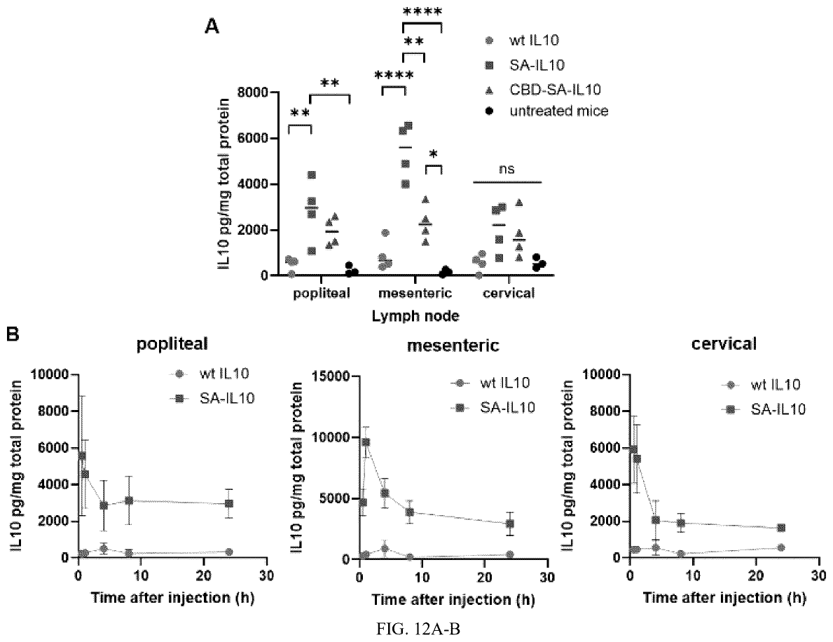

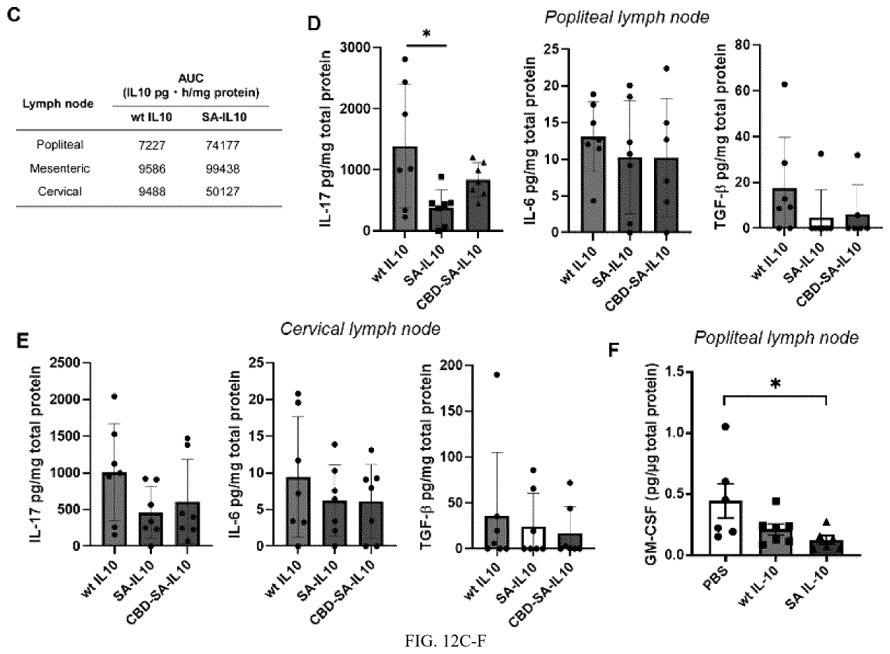

SA-fused IL-10 showed micromolar affinity to FcRn (FIG. 8B) and accumulation within LNs after intravenous injection (FIG. 8D). Next, the amounts of IL-10 and its pharmacokinetics in the LNs were quantitatively evaluated (FIG. 12A-C). After intravenous injection of wt IL-10, SA-IL-10, or CBD-SA-IL-10 within CAIA mice, IL-10 concentrations in the LNs at various time points were detected using ELISA. SA-IL-10 showed significantly higher IL-10 signals in the joint-draining (popliteal) LN, the mesenteric LN and relatively high signals in a non-draining (cervical) LN compared with wt IL-10 and CBD-SA-IL-10 at 4 hr after injection, and CBD-SA-IL-10 showed higher accumulation than wt IL-10 (FIG. 12A). SA-IL-10-injected mice also showed a peak for IL-10 concentration at around 1 hour after injection (FIG. 12B) and 5-10 times higher AUC than wt IL-10 in the LNs (FIG. 12C). These data indicate that SA-IL-10 immediately accumulated within LNs after intravenous injection and showed higher retention in the LNs compared with wt IL-10.

High concentrations and AUC of SA-IL-10 in the LNs may affect the phenotypes of various immune cells in LNs and other secondary lymphoid organs. Therefore, immune cell populations in the spleen and popliteal LN were analyzed by flow cytometry (FIG. 15). Intravenous injection of SA-IL-10 induced a significant decrease in the frequency of CD3⁺T cells and CD45⁺ lymphocytes in the spleen (FIG. 15A). In addition, the frequencies of CD86⁺dendritic cells, granulocytic myeloid-derived suppressor cells (G-MDSCs), and CD86⁺M1 macrophages decreased and that of CD206⁺M2 macrophages increased after injection of SA-IL-10 compared with PBS or wt IL-10. A similar tendency was observed within the popliteal LNs (FIG. 15B). These data suggest that SA-IL-10 suppressed the activity of APCs and simultaneously activated immunosuppressive M2 macrophages. Deactivation of APCs in the LNs, and high accumulation of IL-10 might suppress the activity of Th17 cells, which play a crucial role in the development of RA (22, 23). The inventors measured Th17-relating cytokines (IL-17, IL-6 and TGF-β) in the LNs in the joint-draining (popliteal) and a non-draining (cervical) LN: compared to treatment with wt IL-10, IL-17 was statistically reduced in the popliteal LN after treatment by SA-IL-10, but not statistically by CBD-SA-IL-10, and levels in the cervical LN were not statistically reduced by either IL-10 variant (FIGS. 12D and 12E). Treatment by SA-IL-10 reduced the concentration of GM-CSF in the popliteal LN, whereas wt IL-10 did not (FIG. 12F).

### E. Albumin-Fused IL-10 Suppresses Inflammatory Responses in the Paws

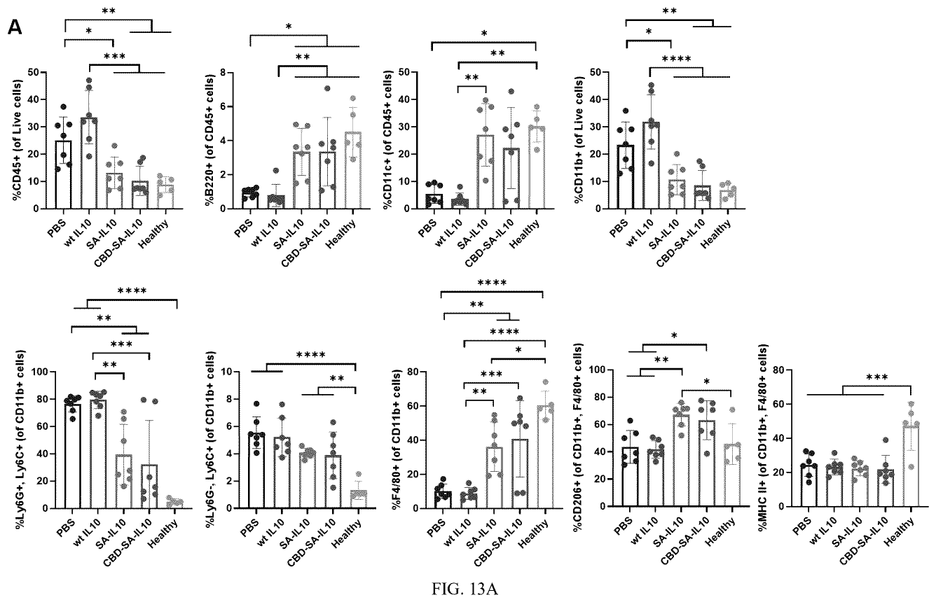

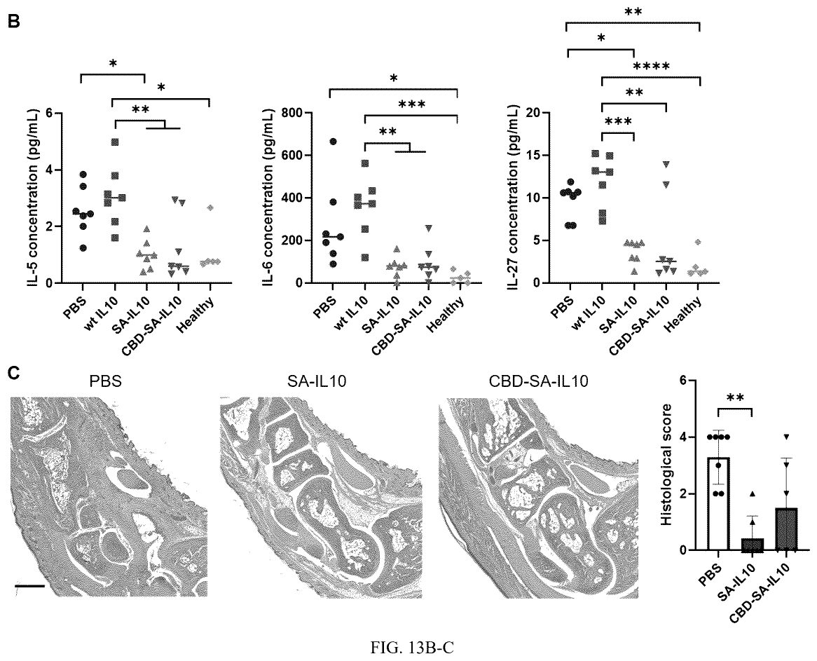

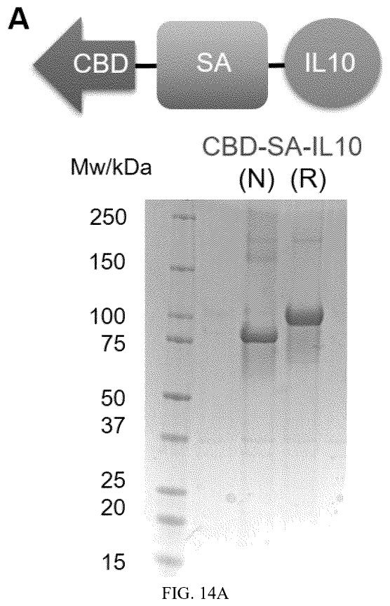

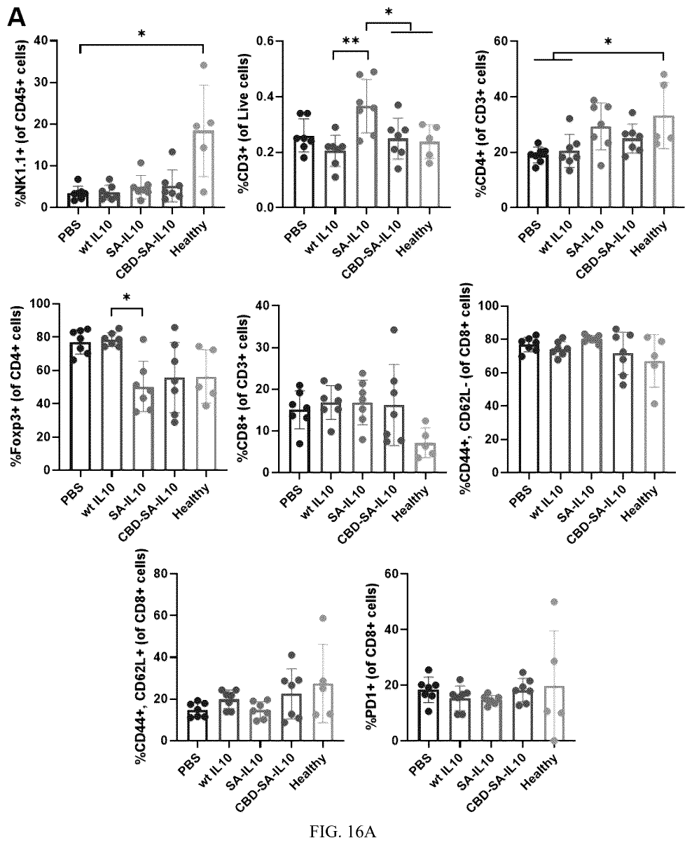

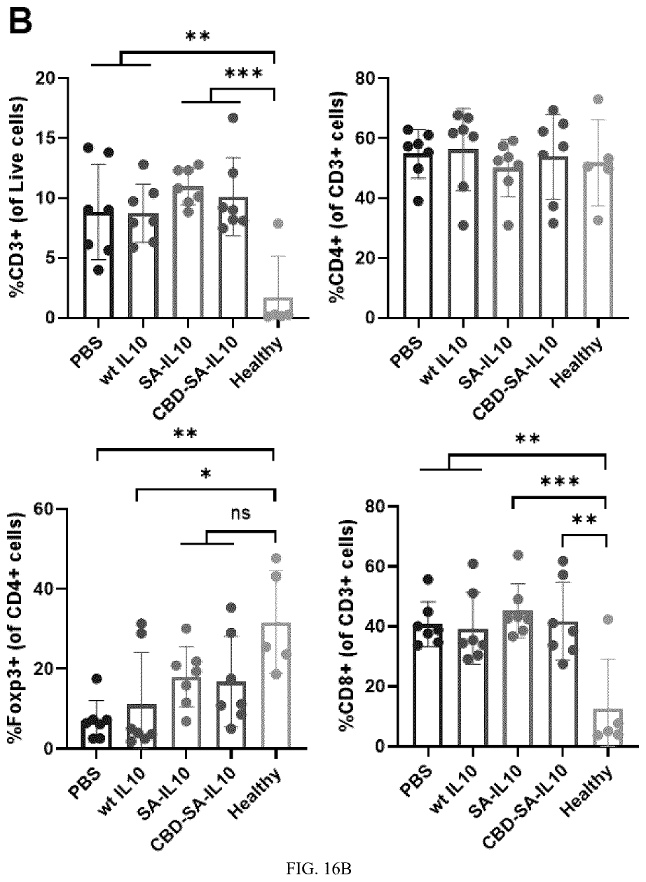

Next, immune cell populations in the hind paws were analyzed using flow cytometry (FIG. 13A). After intravenous injection of SA-IL-10 or CBD-SA-IL-10, frequencies of CD45⁺immune cells were significantly decreased compared with PBS-or wt IL-10-treated groups. Within CD45⁺ cells, the frequencies of B cells and dendritic cells became comparable to levels in healthy mice, and CD11b⁺ cells were remarkably decreased to the level of healthy mice as well. Among CD11b⁺ cells, the G-MDSC population was reduced, and the macrophage frequency was recovered to the level of healthy mice. In addition, the frequency of CD206⁺M2 macrophages was significantly increased by injection of SA-IL-10 compared with PBS or wt IL-10 treatment, even exceeding that of healthy mice. The analysis of T cell populations in the paws revealed that SA-IL-10 suppressed the change in CD4⁺ cells and Foxp3⁺ Treg in CAIA mice (FIG. 16A). Furthermore, SA-IL-10 suppressed a decrease of the Treg frequency in the blood (FIG. 16B). Reflecting these changes in immune cell populations, various inflammatory cytokines in the paws were significantly decreased by intravenous injection of SA-IL-10 or CBD-SA-IL-10, which levels were comparable with those in healthy mice (FIG. 13B). From histological analysis, intravenous administration of SA-IL-10 significantly suppressed the inflammatory responses in the paws compared with PBS-treated mice and reduced joint pathology (FIG. 13C).

F. Albumin-Fused IL-10 Shows No Toxicity after Injection

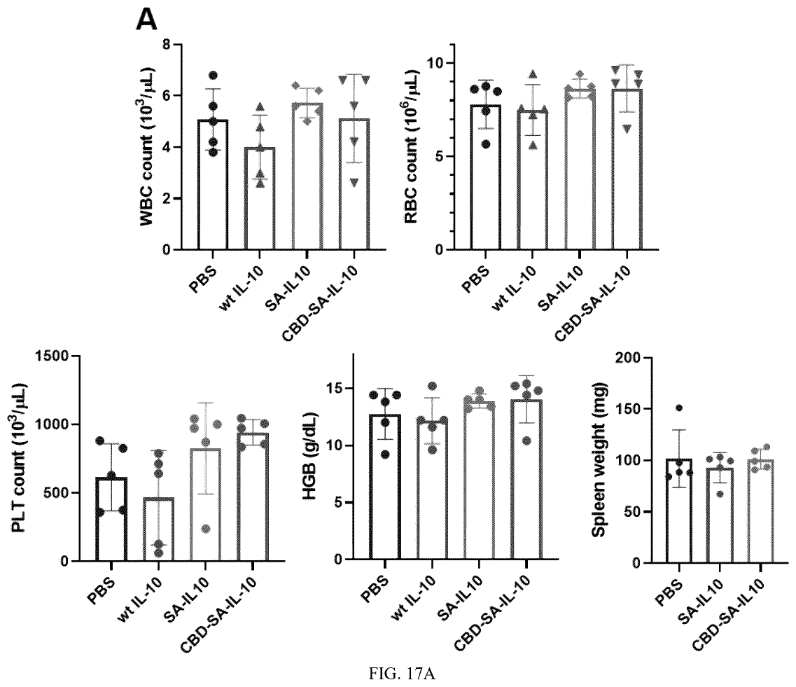

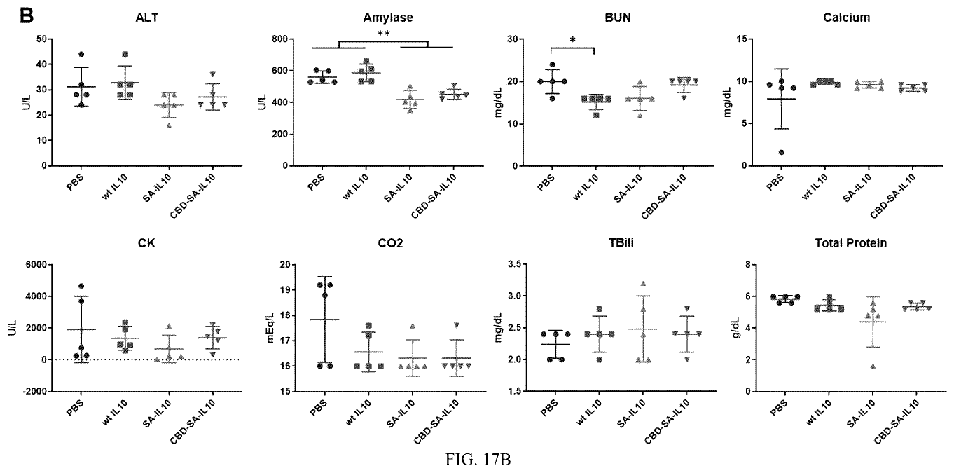

Finally, safety assessments were performed to investigate whether engineered IL-10 demonstrates any adverse effects. Representative blood parameters measured by a hematology analyzer and spleen weights did not show any significant changes among the treatment groups (FIG. 17A). Various biochemical markers in serum were also investigated using a biochemistry analyzer (FIG. 17B). For the engineered IL-10-treated groups, most markers, except for amylase (which was not increased, rather slightly decreased), showed similar levels compared with PBS-treated group, indicating that engineered IL-10 possesses high safety after systemic administration.

### G. Discussion

Current treatment for RA is based on symptomatic therapy by relieving pain, controlling synovitis and suppressing joint injury. Antibody drugs or competitive soluble receptors that neutralize inflammatory cytokines, especially TNF-α, have provided high therapeutic efficacy for patients with RA (4). These biotherapeutics mainly act at the inflamed joints to capture the inflammatory cytokines. However, these inhibitory drugs are known to increase the risk of infection, because their targets are pleiotropic in immune function and these drugs are repeatedly administered to provide their anti-inflammatory effect at disease site (24-27). In addition, administration of antibody drugs can also cause the induction of neutralizing anti-drug antibodies, which decreases therapeutic efficiency (28). Therefore, development of an alternative approach with a structurally different molecular class and with a different immuno-suppressive molecular mechanism such as tolerance is desired.

Here, the inventors explore a novel approach to treat RA through enhanced lymph node trafficking using engineered IL-10, which is a representative anti-inflammatory cytokine and modulates the phenotypes of RA-relating immune cells toward immunosuppressive states. Clinical trials using recombinant IL-10 have been already performed to treat autoimmune diseases including RA (6-8, 29). One of the drawbacks of IL-10 is its short half-life in the blood (8). Here, the inventors genetically fused SA to IL-10 to extend its retention in the blood and moreover in the secondary lymphoid organs. Furthermore, to enhance binding at inflammatory sites, where the microvasculature is hyperpermeable, the inventors genetically fused a CBD derived from the blood protein von Willebrand Factor (16, 17). The inventors evaluated both SA-IL-10 and CBD-SA-IL-10, in comparison to wt IL-10, for amelioration of arthritis in two models. CAIA is a macrophage-and neutrophil-mediated acute RA model, whereas CIA is a T cell-, and especially Th17-mediated RA model. Given that RA is a heterogeneous disease in the clinic and the models complement each other, it is encouraging to show and SA-fused IL-10 suppresses disease severity in both models. SA fusion to IL-10 is crucial for obtaining a marked therapeutic effect, which was comparable to treatment with αTNF-α antibody, a common therapy in the clinic. Additionally, to the inventors' best knowledge, this study is the first to show a therapeutic effect of IL-10 in the CAIA model.

SA fusion to IL-10 resulted in enhanced accumulation within LNs after intravenous injection and kept high IL-10 concentrations in the LNs for prolonged durations compared with wt IL-10 (FIG. 12A). So far, LN trafficking of SA or albumin-binding nanoparticles has been mainly achieved by intradermal or subcutaneous administration, where the LN is accessed via the afferent lymphatic vessel downstream of the collecting lymphatics at the injection site (30-33). In the context of a biodistribution study of an inflammatory cytokines, one paper showed high localization of human SA-fused IL-2 after intravenous injection into spleen, liver and LNs, where IL-2 receptor-expressing T cells exist, but the precise mechanism for this high localization has not been elucidated (34). Here, the inventors reveal enhanced trafficking of SA-fused IL-10 into LNs after intravenous injection, where the SA enters the LN via the blood vasculature. CBD-SA-IL-10 showed lesser accumulation in the LNs than SA-IL-10, perhaps due to binding to collagen in other tissues. Both SA-IL-10 and CBD-SA-IL-10 exhibited high binding affinity (micromolar Ka, as expected) to FcRn (FIGS. 8B and 14B), which provides prolonged blood circulation properties to both proteins via recycling mediated via FcRn expressed in vascular endothelial cells (FIG. 9A). Recycling via transcytosis (from the basal side back to the lumenal side) of IgG via FcRn is a well-established phenomenon, whereas the same phenomenon for SA has been more recently reported (19, 20). Here, in the LN, molecular transport would seem to be in the other direction, from the lumenal to the basal side. Interestingly, histological analysis revealed the accumulation of SA-IL-10 surrounding the HEVs of the LNs (FIG. 8D). Further experiments are required to reveal a more detailed mechanism of enhanced LN accumulation and the relationship with FcRn, such as LN accumulation analysis using mutated SA-fused IL-10 to abrogate FcRn binding.

SA-IL-10 shows high binding to APCs (FIG. 8B). After accumulation within the LNs, SA-IL-10 molecules are taken up by APCs resident within the LNs, leading to the suppression of dendritic cell and M1 macrophage activities and the induction of M2 macrophages (FIG. 15). M2 macrophages can change the differentiation fate of ThO cells to Treg cells in the LNs (35). Furthermore, the immunosuppressive environment by high concentrations of IL-10 in the LN may cause the further polarization of macrophages to M2 phenotype and the suppression of Th17 differentiation (36, 37), resulting in the decrease of IL-17, GM-CSF or other cytokines in the LNs that the inventors observed (FIGS. 12D and 12F). GM-CSF is a cytokine that is a marker for pathogenic Th17, and its inhibitory antibody is currently being tested in clinical trials (38). Thus, the decrease of GM-CSF by SA-IL-10 treatment indicates decreased immuno-activation in the joint-draining LN. Th17 cells reportedly express IL-10 receptor, and IL-10 binding suppresses IL-17 expression and secretion (14, 36). Because Th17 cell antigen recognition primarily occurs in the lymphoid tissue, SA-IL-10 may bind to Th17 cells directly to suppress the IL-17 pathway. These changes in LNs also suppressed the infiltration of immune cells, especially G-MDSC and macrophages, into the paws (FIG. 13A) and also induced an increase of M2 macrophages (FIG. 13A), resulting in the decrease of inflammatory cytokines (FIG. 13B) and the suppression of joint inflammation (FIGS. 10, 11, and 13C).

SA-IL-10 induced high anti-inflammatory responses after administration by any of the routes tested, namely intravenous, subcutaneous (at a distant site) or footpad (local) injections, suggesting that SA-IL-10 can enter the LNs systemically after uptake by a local injection-site draining lymphatics and transit through the lymphatics back into the systemic circulation via the thoracic duct. The high therapeutic effect by subcutaneous injection suggests a particular clinical benefit of SA-IL-10. Intravenous CBD-SA-IL-10 also shows suppression effects on the development of CAIA and CIA that are comparable to anti-TNF-α antibody, which is the current standard biological therapeutic for RA. However, the therapeutic effects of CBD-SA-IL-10 are lower than that observed with SA-IL-10, which corresponds to lower LN trafficking of CBD-SA-IL-10 (FIG. 12A). CBD-SA-IL-10 might bind to collagens in inflamed tissues after permeation there, which causes the accumulation to inflamed paws (FIG. 9B), but such collagen affinity may interfere the LN trafficking of CBD-SA-IL-10 (16, 39). Therefore, SA-fusion is a simple but effective way for preparation of engineered cytokines to achieve enhanced LN trafficking.

In this study, SA-fusion to IL-10 achieved increased persistence within the LNs, where autoimmunity-relating immune recognition develops and persists. As a result, SA-IL-10 suppressed the main inflammatory pathway of RA progression, yet without inhibition of a pleiotropic inflammatory cytokine such as TNF-α. In addition, SA-IL-10 did not show any remarkable toxicities in preliminary safety assessments (FIG. 17). SA-IL-10 exhibited marked therapeutic effects in both CIA and CAIA models. Therefore, the data suggest the potential of SA-IL-10 for clinical application in suppression of RA, and the inventors' findings more broadly suggest the ability to modulate immunity through systemic tolerogenic manipulation of the LNs in other autoimmune and inflammatory diseases.

H. Amino Acid Sequences for mouse wt IL-10, SA-IL-10 and CBD-SA-IL-10

| | |
|---|---|
| Mouse wt IL-10 | RGQYSREDNNCTHFPVGQSHMLLELRTAFSQVKTFFQTKDQLD |
| | NILLTDSLMQDFKGYLGCQALSEMIQFYLVEVMPQAEKHGPEIK |
| | EHLNSLGEKLKTLRMRLRRCHRFLPCENKSKAVEQVKSDFNKL |
| | QDQGVYKAMNEFDIFINCIEAYMMIKMKSHHHHHH (SEQ ID NO: 53) |
| Mouse serum | EAHKSEIAHRYNDLGEQHFKGLVLIAFSQYLQKCSYDEHAKLVQ |
| albumin and | EVTDFAKTCVADESAANCDKSLHTLFGDKLCAIPNLRENYGELA |
| mouse IL-10 | DCCTKQEPERNECFLQHKDDNPSLPPFERPEAEAMCTSFKENPTT |
| fusion protein | FMGHYLHEVARRHPYFYAPELLYYAEQYNEILTQCCAEADKES |
| | CLTPKLDGVKEKALVSSVRQRMKCSSMQKFGERAFKAWAVAR |
| | LSQTFPNADFAEITKLATDLTKVNKECCHGDLLECADDRAELAK |
| | YMCENQATISSKLQTCCDKPLLKKAHCLSEVEHDTMPADLPAIA |
| | ADFVEDQEVCKNYAEAKDVFLGTFLYEYSRRHPDYSVSLLLRLA |
| | KKYEATLEKCCAEANPPACYGTVLAEFQPLVEEPKNLVKTNCDL |
| | YEKLGEYGFQNAILVRYTQKAPQVSTPTLVEAARNLGRVGTKC |
| | CTLPEDQRLPCVEDYLSAILNRVCLLHEKTPVSEHVTKCCSGSLV |
| | ERRPCFSALTVDETYVPKEFKAETFTFHSDICTLPEKEKQIKKQTA |
| | LAELVKHKPKATAEQLKTVMDDFAQFLDTCCKAADKDTCFSTE |
| | GPNLVTRCKDALAGGGSGGGSSRGQYSREDNNCTHFPVGQSHM |
| | LLELRTAFSQVKTFFQTKDQLDNILLTDSLMQDFKGYLGCQALS |
| | EMIQFYLVEVMPQAEKHGPEIKEHLNSLGEKLKTLRMRLRRCHR |
| | FLPCENKSKAVEQVKSDFNKLQDQGVYKAMNEFDIFINCIEAYM |
| | MIKMKSHHHHHH (SEQ ID NO: 54) |
| Human VWF | CSQPLDVVLLLDGSSSLPESSFDKMKSFAKAFISKANIGPHLTQVS |
| A3 domain | VIQYGSINTIDVPWNVVQEKAHLQSLVDLMQQEGGPSQIGDALA |
| (CBD), mouse | FAVRYVTSQIHGARPGASKAVVIIIMDTSLDPVDTAADAARSNR |
| serum albumin | VAVFPVGVGDRYDEAQLRILAGPGASSNVVKLQQVEDLSTMAT |
| and mouse IL- | LGNSFFHKLCSGFSGVGGGSGGGSEAHKSEIAHRYNDLGEQHFK |
| 10 fusion | GLVLIAFSQYLQKCSYDEHAKLVQEVTDFAKTCVADESAANCD |
| protein | KSLHTLFGDKLCAIPNLRENYGELADCCTKQEPERNECFLQHKD |
| | DNPSLPPFERPEAEAMCTSFKENPTTFMGHYLHEVARRHPYFYA |
| | PELLYYAEQYNEILTQCCAEADKESCLTPKLDGVKEKALVSSVR |
| | QRMKCSSMQKFGERAFKAWAVARLSQTFPNADFAEITKLATDL |
| | TKVNKECCHGDLLECADDRAELAKYMCENQATISSKLQTCCDK |
| | PLLKKAHCLSEVEHDTMPADLPAIAADFVEDQEVCKNYAEAKD |
| | VFLGTFLYEYSRRHPDYSVSLLLRLAKKYEATLEKCCAEANPPA |
| | CYGTVLAEFQPLVEEPKNLVKTNCDLYEKLGEYGFQNAILVRYT |
| | QKAPQVSTPTLVEAARNLGRVGTKCCTLPEDQRLPCVEDYLSAI |
| | LNRVCLLHEKTPVSEHVTKCCSGSLVERRPCFSALTVDETYVPK |
| | EFKAETFTFHSDICTLPEKEKQIKKQTALAELVKHKPKATAEQLK |
| | TVMDDFAQFLDTCCKAADKDTCFSTEGPNLVTRCKDALAGGGS |
| | GGGSSRGQYSREDNNCTHFPVGQSHMLLELRTAFSQVKTFFQTK |
| | DQLDNILLTDSLMQDFKGYLGCQALSEMIQFYLVEVMPQAEKH |
| | GPEIKEHLNSLGEKLKTLRMRLRRCHRFLPCENKSKAVEQVKSD |
| | FNKLQDQGVYKAMNEFDIFINCIEAYMMIKMKSHHHHHH |
| | (SEQ ID NO: 55) |

### I. Materials and Methods

### 1. Study Design

This study was designed to test the strategy of targeting anti-inflammatory cytokines to LNs through engineered affinity for FcRn. Specifically, the inventors tested whether LN targeting of the anti-inflammatory cytokine IL-10 by serum albumin fusion is superior to nontargeted wt IL-10 and currently available anti-inflammatory antibody therapeutics (αTNF-α) in mouse models of RA. To evaluate the efficacy of wt IL-10, SA-IL-10 and anti-TNF-α antibody, the inventors scored arthritic symptoms and joint histology in both the passive CAIA model and the active CIA model. The inventors also measured biodistribution and LN trafficking, immune responses within LNs and paws, and various aspects of toxicity after treatment. Statistical methods were not used to predetermine necessary sample size, but sample sizes were chosen on the basis of estimates from pilot experiments so that appropriate statistical tests could yield statistically significant results. Production of wt IL-10, SA-IL-10 and CBD-SA-IL-10 was performed by multiple individuals to ensure reproducibility. All experiments were replicated at least twice. For animal studies, mice were randomized into treatment groups within a cage immediately before the first drug injection and treated in the same manner. The n values used to calculate statistics are indicated in the figure legends. Drug administration and pathological analyses were performed in a blinded fashion. Statistical methods are described in the “Statistical Analysis” section.

### 2. Production and Purification of Recombinant Proteins

The sequences encoding for the mouse serum albumin without pro-peptide (25 to 608 amino acids of whole serum albumin), mouse IL-10, human VWF A3 domain residues Cys1670-Glyl874 (907-1111 of mature VWF, referred to herein as CBD), and a (GGGS) 2 linker were synthesized and subcloned into the mammalian expression vector pcDNA3.1 (+) by Genscript. A sequence encoding for 6 His was added at the C-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×10⁶ cells/mL. 2 μg/mL plasmid DNA, 2 μg/mL linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO₂. Seven days after transfection, the cell culture medium was collected by centrifugation and filtered through a 0.22 μm filter. Culture media was loaded into a HisTrap HP 5 mL column (GE Healthcare), using an ÄKTA pure 25 (GE Healthcare). After washing the column with wash buffer (20 mM NaH₂PO₄, 0.5 M NaCl, pH 8.0), protein was eluted with a gradient of 500 mM imidazole (in 20 mM NaH₂PO₄, 0.5 M NaCl, pH 8.0). The protein was further purified with size exclusion chromatography using a HiLoad Superdex 200PG column (GE Healthcare) using PBS as an eluent. All purification steps were carried out at 4° C. The expression of the proteins was verified as >90% pure by SDS-PAGE. Purified proteins were tested for endotoxin via HEK-Blue TLR4 reporter cell line and endotoxin levels were confirmed to be less than 0.01 EU/mL. Protein concentration was determined through absorbance at 280 nm using NanoDrop (Thermo Scientific).

### 3. Detection of Binding to Collagens and FcRn

SPR measurements were carried out with Biacore X100 instrument. In collagen binding experiments, recombinant human type I or type III collagen (Millipore Sigma) were immobilized on a CM5 sensor chip by standard amine coupling method (˜1500 resonance units (RUs)) and blocked with ethanolamine. A reference cell was also blocked with ethanolamine. Binding assays were carried out at room temperature and the Ka values of the CBD-SA-IL-10 were determined by fitting 1:1 Langmuir binding model to the data using BIAevaluation software (GE Healthcare). In FcRn binding experiments, recombinant mouse FcRn (Acro Biosystems) was immobilized via amine coupling on a C1 chip (GE Healthcare) for ˜200 RU according to the manufacturer's instructions. SA-IL-10 or CBD-SA-IL-10 was flowed at decreasing concentrations in the running buffer (0.01 M monobasic anhydrous sodium phosphate, pH 5.8, 0.15 M NaCl) at 30 μL/min at room temperature. The sensor chip was regenerated with PBS, pH 7.4 for every cycle. Specific binding of SA-fused proteins to FcRn was calculated by comparison to a non-functionalized channel used as a reference. The Ka values of the SA-IL-10 and CBD-SA-IL-10 were determined by fitting 1:1 Langmuir binding model to the data using BIAevaluation software (GE Healthcare).

### 4. Mice

BALB/c female mice at 7 wk of age and DBA/1J male mice at 8 wk of age were obtained from the Jackson Laboratory. Experiments were performed with approval from the Institutional Animal Care and Use Committee of the University of Chicago.

### 5. Binding of the Proteins to Splenocytes or LN-Derived Cells

Single-cell suspensions were obtained by gently disrupting the spleen and popliteal LN through a 70-um cell strainer. Red blood cells were lysed with ACK lysing buffer (Quality Biological) for splenocytes. Cells were counted and re-suspended in RPMI-1640 supplemented with 10% FBS and 1% penicillin/streptomycin (all from Life Technologies). 1×10⁵ cells/well were seeded in a 96 well microplate and were incubated with 2 μg/100 μL of SA, SA-IL-10 or CBD-SA-IL-10 for 30 min on ice. After 4-times washing by PBS, cells were further incubated with anti-mouse albumin antibodies (abcam) for 20 min on ice. After 3-times washing by PBS, cells were incubated with 1 μg/mL AlexaFluor 647-labeled anti-Rabbit IgG (Jackson ImmunoResearch), anti-B220 (RA3-6B2, BioLegend), anti-CD3 (145-2C11, BD Biosciences), anti-CD4 (RM4-5, BD Biosciences), anti-CD8 (53-6.7, BD Biosciences), anti-CD11c (HL3, BD Biosciences), anti-CD45 (30-F11, BD Biosciences) and anti-F4/80 (T45-2342, BD Biosciences) antibodies for 20 min on ice. Cells were analyzed by flow cytometry as described below.

### 6. Plasma Pharmacokinetics of the Proteins

IL-10, SA-IL-10 or CBD-SA-IL-10 (equivalent to 35 μg of IL-10) was injected intravenously into female BALB/c mice. Blood samples were collected in protein-low binding tubes at 1, 5, 10, and 30 min, and 1, 4, 8 and 24 hr after injection, followed by overnight incubation at 4° C. IL-10 concentrations in serum were measured by IL-10 Mouse Uncoated ELISA kit (Invitrogen) according to the manufacturer's protocol. Exponential two-phase decay (Y=Ae^{-αt}+Be^{-βt}) fitting was used to calculate the half-life. Fast clearance half-life, t_{1/2,α}; slow clearance half-life, t_{1/2,β}. Data were analyzed using Prism software (v8, GraphPad).

### 7. CAIA Model

Arthritis was induced in female BALB/c mice by intraperitoneal injection of anti-collagen antibody cocktail (1.0 mg/mouse, Chondrex) on day 0, followed by intraperitoneal injection of LPS (25 μg/mouse, Chondrex) on day 3. Only for FIG. 3B-C, 1.5 mg/mouse of anti-collagen antibody cocktail was injected. On the day 3, mice were intravenously, subcutaneously (mid-back), or via footpad injected with PBS, wt IL-10, SA-IL-10, CBD-SA-IL-10 (each equivalent to 43.5 μg of IL-10), or 200 μg of Rat anti-mouse TNF-x antibody (clone XT3.11, Bio X Cell) before LPS injection. Joint swelling was scored every day according to the manufacture's protocol (Chondrex). On the last day of scoring, the hind paws were fixed in 10% neutral formalin (Sigma-Aldrich), decalcified in Decalcifer II (Leica), and then provided for histological analysis. Paraffin-embedded paws were sliced at 5 μm thickness and stained with H&E. The images were scanned with a Pannoramic digital slide scanner and analyzed using a Pannoramic Viewer software. The severity of synovial hyperplasia and bone resorption for the arthritis model was scored by three-grade evaluation (0-2) according to the previously reported criteria with slight modifications as follows: 0, normal to minimal infiltration of pannus in cartilage and subchondral bone of marginal zone; 1, mild to moderate infiltration of marginal zone with minor cortical and medullary bone destruction; 2, severe infiltration associated with total or near total destruction of joint architecture. The scores in both hind paws were summed for each mouse (score per mouse total, 0-4). The histopathological analyses were performed in a blinded fashion.

### 8. CIA Model

Male DBA/1J mice (8 wk old) were immunized by subcutaneous injection at the base of the tail with bovine collagen/complete Freund's adjuvant (CFA) emulsion (Hooke Kit, Hooke Laboratories). Three weeks later, a booster injection of bovine collagen/incomplete Freund's adjuvant (IFA) emulsion (Hooke Kit, Hooke Laboratories) was performed. After the booster injection, mice were inspected every day, and joint swelling was scored according to the manufacture's protocol (Hooke Laboratories). When showing total score of 2-4 (defined as Day 0), mice were intravenously injected with PBS, SA-IL-10, CBD-SA-IL-10 (each equivalent to 43.5 μg of IL-10), or 200 μg of Rat anti-mouse TNF-x antibody (clone XT3.11, Bio X Cell). On the last day of scoring, hind paws were collected and histological analyses were employed as described above.

### 9. In Vivo Bio-Distribution Study

To make fluorescently labeled protein, wt IL-10, SA-IL-10 and CBD-SA-IL-10 were incubated with 8-fold molar excess of using DyLight 800 NHS ester (Thermo Fisher) for 1 hr at room temperature, and unreacted dye was removed by a Zebaspin spin column (Thermo Fisher) according to the manufacturer's instruction. BALB/c mice were intraperitoneal injected by anti-collagen antibody cocktail (1.0 mg/mouse) on day 0, subsequently 10 μg of LPS was injected to right hind paw on day 3. The following day, 20 μg of DyLight 800-labeled proteins were intravenously injected. After 4 hr, organs harvested from the disease model were imaged with the Xenogen IVIS Imaging System 100 (Xenogen) under the following conditions: f/stop: 2; optical filter excitation 745 nm; excitation 800 nm; exposure time: 5 sec; small binning. Each organ was weighed to normalize the fluorescence signal from each organ.

### 10. LN Microscopy

BALB/c mice were intravenously injected with DyLight594-labeled wt IL-10 (43.5 μg) or SA-IL-10 labeled with equimolar amounts of dye. 24 hr after injection, popliteal LNs were harvested and frozen in dry ice with optimal cutting temperature (OCT) compound. Tissue slices (10 μm) were obtained by cryo-sectioning. The tissues were fixed with 2% paraformaldehyde in PBS for 15 min at room temperature. After washing with PBS-T, the tissues were blocked with 2% BSA in PBS-T for 1 hr at room temperature. The tissues were stained with anti-mouse CD3 antibody (1:100, 145-2C11, BioLegend) or anti-mouse peripheral node addressin (PNAd) antibody (1:200, MECA79, BioLegend) and Alexa Fluor 488 donkey anti-rat (1:400, Jackson ImmunoResearch). The tissues were washed three times and then covered with ProLong gold antifade mountant with 4′,6-diamidino-2-phenylindole (DAPI; Thermo Fisher Scientific). An IX83 microscope (Olympus) was used for imaging with 10× magnification for CD3 staining, and a Leica SP8 3D Laser Scanning Confocal microscope with 20× magnification for PNAd staining. Images were processed using ImageJ software (NIH).

### 11. LN Pharmacokinetics

wt IL-10, SA-IL-10 or CBD-SA-IL-10 (each equivalent to 35 μg of IL-10) was injected intravenously into CAIA mice. Popliteal, mesenteric, cervical LNs were collected at 30 min, and 1, 4, 8 and 24 hr after injection, and were subsequently homogenized using Lysing Matrix D and FastPrep-24 5G (MP Biomedical) for 40 s at 5000 beats/min in T-PER tissue protein extraction reagent (Thermo Scientific) with complete™ proteinase inhibitor cocktail (Roche). After homogenization, samples were incubated overnight at 4° C. Samples were centrifuged (5000 g, 5 min), and the total protein concentration and IL-10 concentration were analyzed using a BCA assay kit (Thermo Fisher) and IL-10 Mouse Uncoated ELISA kit (Invitrogen), respectively. Simultaneously, cytokine levels in the LN extract were measured using Mouse Uncoated ELISA kit (Invitrogen) or Ready-SET-Go! ELISA kits (eBioscience) according to the manufacturer's protocol. For detection of GM-CSF, wt IL-10 or SA-IL-10 (each equivalent to 35 μg of IL-10) was injected intravenously twice with a 3 day interval into CAIA mice. The day following the last injection, popliteal LNs were collected for detection of GM-CSF.

### 12. Flow Cytometry

CAIA mice were intravenously injected with PBS, wt IL-10, SA-IL-10 or CBD-SA-IL-10 (each equivalent to 43.5 μg of IL-10). Eight days after, blood and hind paws were harvested. Red blood cells in blood were lysed with ACK lysing buffer (Quality Biological), followed by antibody staining for flow cytometry. Paws were digested in Dulbecco's Modified Eagle Medium (DMEM) supplemented with 2% FBS, 2 mg/mL collagenase D and 40 μg/mL DNase I (Roche) for 60 min at 37° C. Single-cell suspensions were obtained by gently disrupting through a 70-um cell strainer. Antibodies against the following molecules were used: anti-mouse CD3 (145-2C11, BD Biosciences), CD4 (RM4-5, BD Biosciences), anti-mouse CD8a (53-6.7, BD Biosciences), anti-mouse CD25 (PC61, BD Biosciences), anti-mouse CD45 (30-F11, BD Biosciences), CD44 (IM7, BD Biosciences), CD62L (MEL-14, BD Biosciences), PD-1 (29F.1A12, BD Biosciences), NK1.1 (PK136, BD Biosciences), Foxp3 (MF23, BD Biosciences), F4/80 (T45-2342, BD Biosciences), MHC II (M5/114.15.2, BioLegend), CD206 (C068C2, BioLegend), Ly6G (1A8, BioLegend), Ly6C (HK1.4, BioLegend), CD11b (M1/70, BioLegend), CD11c (HL3, BD Biosciences), B220 (RA3-6B2, BioLegend). Fixable live/dead cell discrimination was performed using Fixable Viability Dye eFluor 455 (eBioscience) according to the manufacturer's instructions. Staining was carried out on ice for 20 min if not indicated otherwise, and intracellular staining was performed using the Foxp3 staining kit according to manufacturer's instructions (BioLegend). Following a washing step, cells were stained with specific antibodies for 20 min on ice prior to fixation. All flow cytometric analyses were done using a Fortessa (BD Biosciences) flow cytometer and analyzed using FlowJo software (Tree Star).

### 13. Safety Assessments

BALB/c mice were intravenously injected with PBS, wt IL-10, SA-IL-10 or CBD-SA-IL-10 (each equivalent to 43.5 μg of IL-10). Two days after injection, blood samples collected from mice were analyzed using a COULTER Ac·T 5diff CP hematology analyzer (Beckman Coulter) according to the manufacturer's instructions. Spleen weight was also measured. Serum samples collected from protein-injected mice were analyzed using Biochemistry Analyzer (Alfa Wassermann Diagnostic Technologies) according to the manufacturer's instructions.

### 14. Statistical Analysis

Statistically significant differences between experimental groups were determined using Prism software (v8, GraphPad). Where one-way ANOVA followed by Tukey's HSD post hoc test was used, variance between groups was found to be similar by Brown-Forsythe test. For non-parametric data, Kruskal-Wallis test followed by Dunn's multiple comparison test was used. For single comparisons, a two-tailed Student's t-test was used. The symbols *, **, *** and indicate P values less than 0.05, 0.01, 0.001 and 0.0001 respectively; ns, not significant.

### J. REFERENCES

The following references and the publications referred to throughout the specification, to the extent that they provide exemplary procedural or other details supplementary to those set forth herein, are specifically incorporated herein by reference.
1. G. S. Firestein, Evolving concepts of rheumatoid arthritis. Nature 423, 356-361 (2003).
2. I. B. McInnes, G. Schett, The pathogenesis of rheumatoid arthritis. N. Engl. J. Med. 365, 2205-2219 (2011).
3. Q. Guo, Y. Wang, D. Xu, J. Nossent, N. J. Pavlos, J. Xu, Rheumatoid arthritis: pathological mechanisms and modern pharmacologic therapies. Bone Res. 6, 15 (2018).
4 M. E. Weinblatt, E. C. Keystone, D. E. Furst, L. W. Moreland, M. H. Weisman, C. A. Birbara, L. A. Teoh, S. A. Fischkoff, E. K. Chartash, Adalimumab, a fully human anti-tumor necrosis factor alpha monoclonal antibody, for the treatment of rheumatoid arthritis in patients taking concomitant methotrexate: The ARMADA trial. Arthritis Rheum. 48, 35-45 (2003).
5 K. N. Couper, D. G. Blount, E. M. Riley, IL-10: The master regulator of immunity to infection. J. Immunol. 180, 5771-5777 (2008).
6. K. Asadullah, W. Sterry, H. D. Volk, Interleukin-10 therapy-review of a new approach. Pharmacol. Rev. 55, 241-269 (2003).
7. W. Ouyang, A. O'Garra, IL-10 family cytokines IL-10 and IL-22: from basic science to clinical translation. Immunity 50, 871-891 (2019).
8 A. Saxena, S. Khosraviani, S. Noel, D. Mohan, T. Donner, A. R. Hamad, Interleukin-10 paradox: A potent immunoregulatory cytokine that has been difficult to harness for immunotherapy. Cytokine 74, 27-34 (2015).
9. H. M. Alvarez, O. Y. So, S. Hsieh, N. Shinsky-Bjorde, H. Ma, Y. Song, Y. Pang, M. Marian, E. Escandón, Effects of PEGylation and immune complex formation on the pharmacokinetics and biodistribution of recombinant interleukin 10 in mice. Drug Metab. Dispos. 40, 360-373 (2012).
10. K. Schwager, M. Kaspar, F. Bootz, R. Marcolongo, E. Paresce, D. Neri, E. Trachsel, Preclinical characterization of DEKAVIL (F8-IL10), a novel clinical-stage immunocytokine which inhibits the progression of collagen-induced arthritis. Arthritis Res. Ther. 11, R142 (2009).
11. F. Doll, K. Schwager, T. Hemmerle, D. Neri, Murine analogues of etanercept and of F8-IL10 inhibit the progression of collagen-induced arthritis in the mouse. Arthritis Res. Ther. 15, R138 (2013).
12. S. T. G. Bruijnen, D. M. S. H. Chandrupatla, L. Giovanonni, D. Neri, D. J. Vugts, M. C. Huisman, O. S. Hoekstra, R. J. P. Musters, A. A. Lammertsma, G. A. M. S. van Dongen, G. Jansen, C. F. M. Molthoff, C. J. van der Laken, F8-IL10: a new potential antirheumatic drug evaluated by a PET-guided translational approach. Mol. Pharm. 16, 273-281 (2019).
13. B. Ruffell, D. Chang-Strachan, V. Chan, A. Rosenbusch, C. M. Ho, N. Pryer, D. Daniel, E. S. Hwang, H. S. Rugo, Macrophage IL10 blocks CD8⁺T cell-dependent responses to chemotherapy by suppressing IL-12 expression in intratumoral dendritic cells. Cancer Cell 26, 623-637 (2014).
14. S. Huber, N. Gagliani, E. Esplugues, W. O'Connor, Jr., F. J. Huber, A. Chaudhry, M. Kamanaka, Y. Kobayashi, C. J. Booth, A. Y. Rudensky, M. G. Roncarolo, M. Battaglia, R. A. Flavell, Th17 cells express interleukin-10 receptor and are controlled by Foxp3- and Foxp3⁺ regulatory CD4⁺ T cells in an interleukin-10-dependent manner. Immunity 34, 554-565 (2011).
15. S. K. Mittal, K. J. Cho, S. Ishido, P. A. Roche, Interleukin 10 (IL-10)-mediated immunosuppression: MARCH-I induction regulates antigen presentation by macrophages but not dendritic cells. J. Biol. Chem. 290, 27158-27167 (2015).
16. J. Ishihara, A. Ishihara, K. Sasaki, S. S. Lee, J. M. Williford, M. Yasui, H. Abe, L. Potin, P. Hosseinchi, K. Fukunaga, M. M. Raczy, L. T. Gray, A. Mansurov, K. Katsumata, M. Fukayama, S. J. Kron, M. A. Swartz, J. A. Hubbell, Targeted antibody and cytokine cancer immunotherapies through collagen affinity. Sci. Transl. Med. 11, eaau3259 (2019).
17. K. Sasaki, J. Ishihara, A. Ishihara, R. Miura, A. Mansurov, K. Fukunaga, J. A. Hubbell, Engineered collagen-binding serum albumin as a drug conjugate carrier for cancer therapy. Sci. Adv. 5, eaaw6081 (2019).
18. M. Miyasaka, T. Tanaka, Lymphocyte trafficking across high endothelial venules: dogmas and enigmas. Nat. Rev. Immunol. 4, 360-370 (2004).
19. M. Pyzik, T. Rath, T. T. Kuo, S. Win, K. Baker, J. J. Hubbard, R. Grenha, A. Gandhi, T. D. Krämer, A. R. Mezo, Z. S. Taylor, K. McDonnell, V. Nienaber, J. T. Andersen, A. Mizoguchi, L. Blumberg, S. Purohit, S. D. Jones, G. Christianson, W. I. Lencer, I. Sandlie, N. Kaplowitz, D. C. Roopenian, R. S. Blumberg, Hepatic FcRn regulates albumin homeostasis and susceptibility to liver injury. Proc. Natl. Acad. Sci. USA 114, E2862-E2871 (2017).
20. M. Pyzik, K. M. K. Sand, J. J. Hubbard, J. T. Andersen, I. Sandlie, R. S. Blumberg, The neonatal Fc receptor (FcRn): a misnomer? Front. Immunol. 10, 1540 (2019).
21. K. Katsumata, J. Ishihara, A. Mansurov, A. Ishihara, M. M. Raczy, E. Yuba, J. A. Hubbell, Targeting inflammatory sites through collagen affinity enhances the therapeutic efficacy of anti-inflammatory antibodies. Sci. Adv. 5, eaay 1971 (2019)
22. S. Kotake, T. Yago, T. Kobashigawa, Y. Nanke, The plasticity of Th17 cells in the pathogenesis of rheumatoid arthritis. J. Clin. Med. 6, E67 (2017).
23. J. P. van Hamburg, S. W. Tas, Molecular mechanisms underpinning T helper 17 cell heterogeneity and functions in rheumatoid arthritis. J. Autoimmun. 87, 69-81 (2018).
24. Y. C. Chiang, L. N. Kuo, Y. H. Yen, C. H. Tang, H. Y. Chen, Infection risk in patients with rheumatoid arthritis treated with etanercept or adalimumab. Comput. Methods Programs Biomed. 116, 319-327 (2016).
25. C. Downey, Serious infection during etanercept, infliximab and adalimumab therapy for rheumatoid arthritis: A literature review. Int. J. Rheum. Dis. 19, 536-550 (2016).
26. J. D. Reid, B. Bressler, J. English, A case of adalimumab-induced pneumonitis in a 45-year-old man with Crohn's disease. Can. Respir. J. 18, 262-264 (2011).
27. J. Zalevsky, A. K. Chamberlain, H. M. Horton, S. Karki, I. W. Leung, T. J. Sproule, G. A. Lazar, D. C. Roopenian, J. R. Desjarlais. Enhanced antibody half-life improves in vivo activity. Nat. Biotechnol. 28, 157-159 (2010).
28. I. Cludts, F. R. Spinelli, F. Morello, J. Hockley, G. Valesini, M. Wadhwa, Anti-therapeutic antibodies and their clinical impact in patients treated with the TNF antagonist adalimumab. Cytokine 96, 16-23 (2017).
29. X. Wang, K. Wong, W. Ouyang, S. Rutz, Targeting IL-10 family cytokines for the treatment of human diseases. Cold Spring Harb. Perspect. Biol. 11, a028548 (2019).
30. H. Liu, K. D. Moynihan, Y. Zheng, G. L. Szeto, A. V. Li, B. Huang, D. S. Van Egeren, C. Park, D. J. Irvine, Structure-based programming of lymph-node targeting in molecular vaccines. Nature 507, 519-522 (2014).
31. P. Wang, P. Zhao, S. Dong, T. Xu, X. He, M. Chen, An albumin-binding polypeptide both targets cytotoxic T lymphocyte vaccines to lymph nodes and boosts vaccine presentation by dendritic cells. Theranostics 8, 223-236 (2018).
32. G. Zhu, G. M. Lynn, O. Jacobson, K. Chen, Y. Liu, H. Zhang, Y. Ma, F. Zhang, R. Tian, Q. Ni, S. Cheng, Z. Wang, N. Lu, B. C. Yung, Z. Wang, L. Lang, X. Fu, A. Jin, I. D. Weiss, H. Vishwasrao, G. Niu, H. Shroff, D. M. Klinman, R. A. Seder, X. Chen, Albumin/vaccine nanocomplexes that assemble in vivo for combination cancer immunotherapy. Nat. Commun. 8, 1954 (2017).
33. K. Vandoorne, Y. Addadi, M. Neeman, Visualizing vascular permeability and lymphatic drainage using labeled serum albumin. Angiogenesis 13, 75-85 (2010).
34. Z. Yao, W. Dai, J. Perry, M.W. Brechbiel, C. Sung, Effect of albumin fusion on the biodistribution of interleukin-2. Cancer Immunol. Immunother. 53, 404-410 (2004).
35. J. Li, H. C. Hsu, J. D. Mountz, The dynamic duo-inflammatory M1 macrophages and Th17 cells in rheumatic diseases. J. Orthop. Rheumatol. 1, 4 (2013).
36. B. Guo, IL-10 modulates Th17 pathogenicity during autoimmune diseases. J. Clin. Cell Immunol. 7, 400 (2016).
37. Y. Degboe, B. Rauwel, M. Baron, J. F. Boyer, A. Ruyssen-Witrand, A. Constantin, J. L. Davignon, Polarization of rheumatoid macrophages by TNF targeting through an IL-10/STAT3 mechanism. Front. Immunol. 10, 3 (2019).
38. A. B. Avci, E. Feist, G. R. Burmester, Tagering GM-CSF in rheumatoid arthritis. Clin. Exp. Rheumatol. 34, S39-S44 (2016). S. Schwager, M. Detmar, Inflammation and lymphatic function. Front. Immunol. 10, 308 (2019).

### Example 3: Prolonged Residence of Albumin-Fused IL-4 in the Secondary Lymphoid Organs Via FcRn Ameliorates Experimental Autoimmune Encephalomyelitis

Multiple sclerosis (MS) is a common and severe demyelinating autoimmune disease of the central nervous system. Although interleukin (IL)-4 suppresses development of pathology in a murine MS model, experimental autoimmune encephalomyelitis (EAE), IL-4 has not been clinically translated. Here, the inventors have engineered a fusion protein of serum albumin (SA) and IL-4 (SA-IL-4) to target secondary lymphoid organs (SLOs), where antigen-specific T cell priming primarily occurs. SA-IL-4 showed greater accumulation and residence time in lymph nodes (LNs) and spleen compared to wild-type (wt) IL-4 via neonatal Fc receptor (FcRn) binding. Subcutaneous administration of SA-IL-4 prevented EAE disease development in all mice and demonstrated higher therapeutic efficacy compared to FTY720 and wt IL-4. SA-IL-4 prevented immune cell infiltration into the spinal cord, facilitating maintenance of spinal cord structure and resulting neurological function. SA-IL-4 decreased integrin expression in antigen-reactive CD4⁺T cells, indicating impaired cell migration capability. SA-IL-4 increased the number of and programmed death ligand-1 expression on granulocyte-like myeloid-derived suppressor cells, a key EAE disease suppressor, in the spinal cord-draining LN (dLN). SA-IL-4 decreased the number of Th17 cells, a pathogenic cell population for EAE disease. In the chronic phase of EAE, SA-IL-4 also showed marked therapeutic effects, accompanied by inhibition of immune cell infiltration into the spinal cord and complete abrogation of immune response to the myelin antigen in the spleen. Engineered SA-IL-4 demonstrates translational promise for MS as both a preventative and therapeutic treatment via accumulation in SLOs.

Multiple sclerosis (MS) is a potentially disabling autoimmune disease that affects millions globally. Autoreactive immune cells home to the central nervous system (CNS) and cause demyelination and consequently focal damage to white matter (1). Lymphocytes and macrophages that have infiltrated into the CNS cause axonal damage. Recent studies have shown that Th17 cells, activated in the secondary lymphoid organs (SLOs), migrate to the spinal cord and brain and play a crucial role in the disease development and severity of MS (20). Thus, inhibition of lymphocyte migration to the CNS and inducing an immune-suppressive microenvironment in the SLOs would provide an effective therapy for MS. FTY720 and anti-integrin α₄ antibody are used in the clinic to inhibit lymphocyte migration (3,4). Experimental autoimmune encephalomyelitis (EAE) is a widely accepted murine model of MS, reflecting many features of disease progression and developmental mechanism, including lymphocyte migration to the CNS and demyelination.

### A. SA-IL-4 Binds to Immune Cells and Inhibits Th17 Differentiation

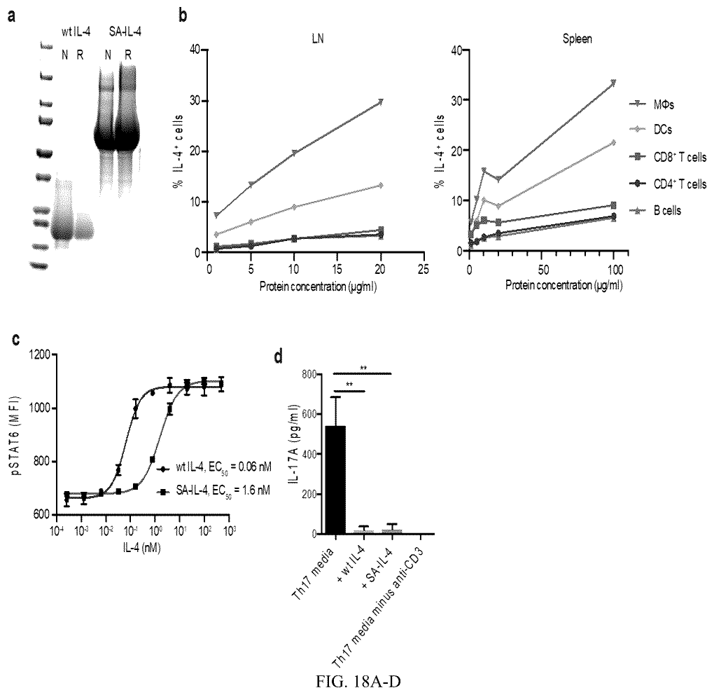

The inventors have recombinantly expressed wild-type (wt) mouse IL-4 and mouse SA-fused mouse IL-4 (FIG. 18A). SDS-PAGE revealed that the molecular size was increased by SA fusion to IL-4. When added to freshly isolated immune cells from LN and spleen, SA-IL4 preferentially bound to antigen presenting cells (APCs), such as macrophages and dendritic cells (DCs) in vitro, compared to other immune cells (FIG. 18B).

IL-4 receptor is expressed on T cells when stimulated (12). SA-IL-4 induced downstream phosphorylation of STAT6 in T cells with 32 times higher EC50 than wt IL-4. This suggests that wt IL-4 is more active than SA-IL-4 in vitro (FIG. 18C). The inventors found that both wt IL-4 and SA-IL-4 inhibit Th17 differentiation of naïve CD4⁺T cells cultured in Th17 cell differentiation media (FIG. 18D). Taken together, the results demonstrate that the inventors successfully made functionally active SA-IL-4 fusion protein.

SA-IL-4 increases blood half-life and persistence in the SLOs, both in LNs and spleen

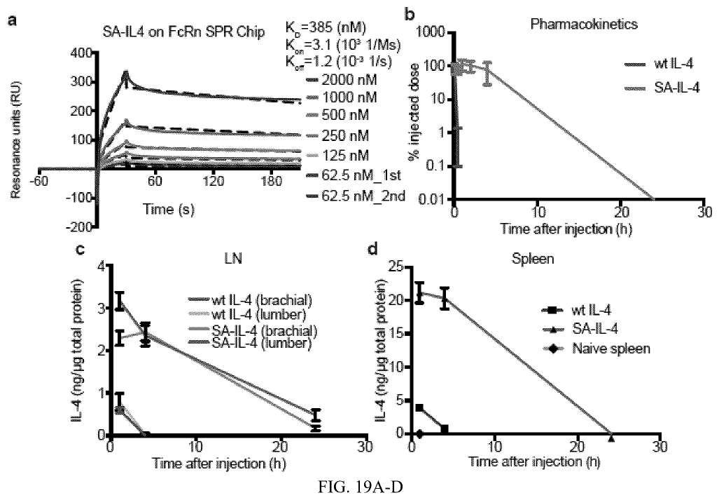

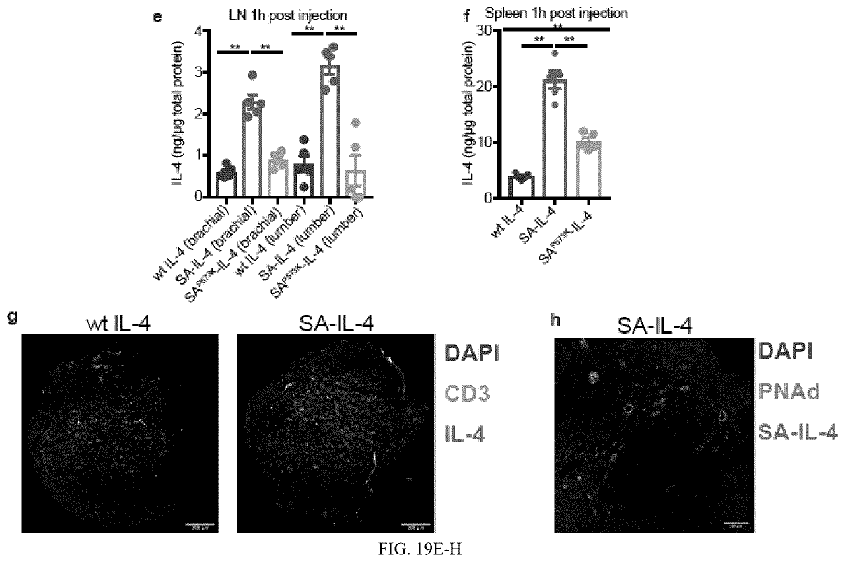

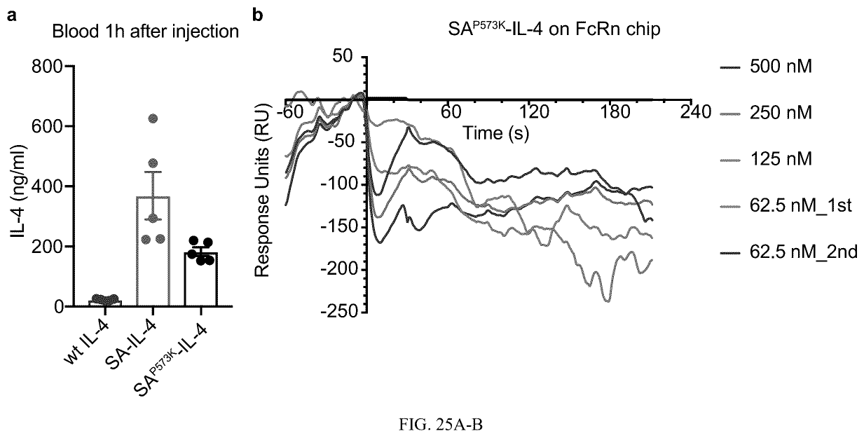

Surface plasmon resonance (SPR) analysis showed that SA-IL-4 binds to FcRn with a dissociation constant (KD) of 385 nM (FIG. 19A). Plasma half-life after intravenous (i.v.) injection of SA-IL-4 was extended remarkably in contrast to wt IL-4, which was cleared from plasma within a few minutes (FIG. 19B). The inventors next tested if i.v.-injected SA-IL-4 accumulates in the spleen and LN using naïve mice. SA-IL-4 substantially increased the amount of IL-4 in both the lumbar and brachial LNs and in the spleen after i.v. injection (FIG. 2cd). Fluorescence-based biodistribution analysis also showed enhanced SA-IL-4 accumulation in the lumbar LN compared to wt IL-4 (FIG. 24). To test the involvement of FcRn in SA-IL-4 accumulation in the LN, the inventors made a P573K point mutation of SA in the fusion to IL-4, which abolishes FcRn binding (FIG. 25A) (13). The SA (P573K) mutation decreased the IL-4 amount in the LN compared to SA-IL-4, down to a similar level as wt IL-4 (FIG. 19E). SA (P573K)-IL-4 has a longer blood half-life compared to wt IL-4 due to an increase in molecular size, but shorter than SA-IL-4 due to impaired FcRn binding (FIG. 25B). Taken together, these data suggest that SA trafficking to the LN requires FcRn binding.

After i.v. injection of SA-IL-4, histology of the LN showed an increased signal of SA-IL-4 in the lumbar LN compared to wt IL-4. SA-IL-4 localized especially to the subcapsular space, without co-localizing with CD3⁺T cells (FIG. 19G). Higher magnification revealed that SA-IL-4 partially co-localized with high endothelial venues, which are indicated by peripheral node addressin⁺ (PNAd⁺) cells (FIG. 19H). The SA (P573K)-IL-4 amount in the spleen was lower than with SA-IL-4 (FIG. 19F). Taken together, the inventors have shown that SA fusion to IL-4 increases persistence of IL-4 within the LN and spleen through FcRn binding.

### B. SA-IL-4 Treatment Strongly Suppresses EAE Disease Development in Prophylactic Treatment

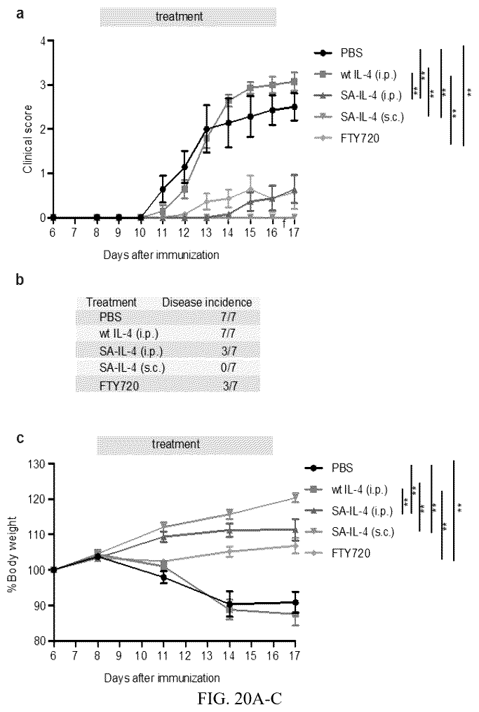

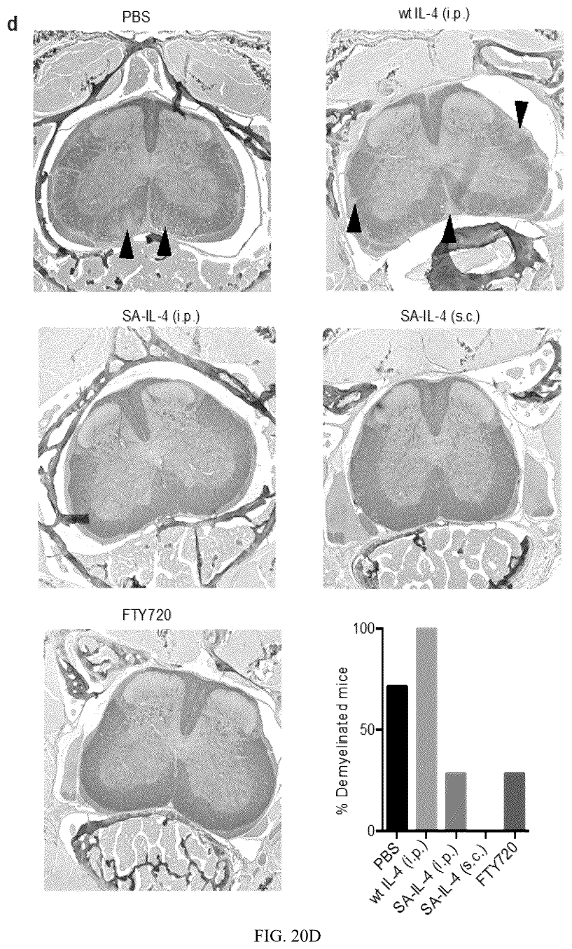

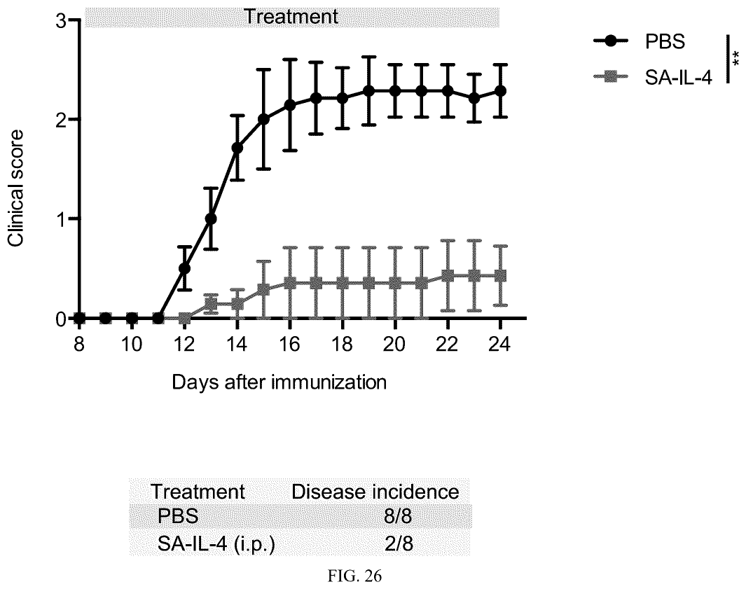

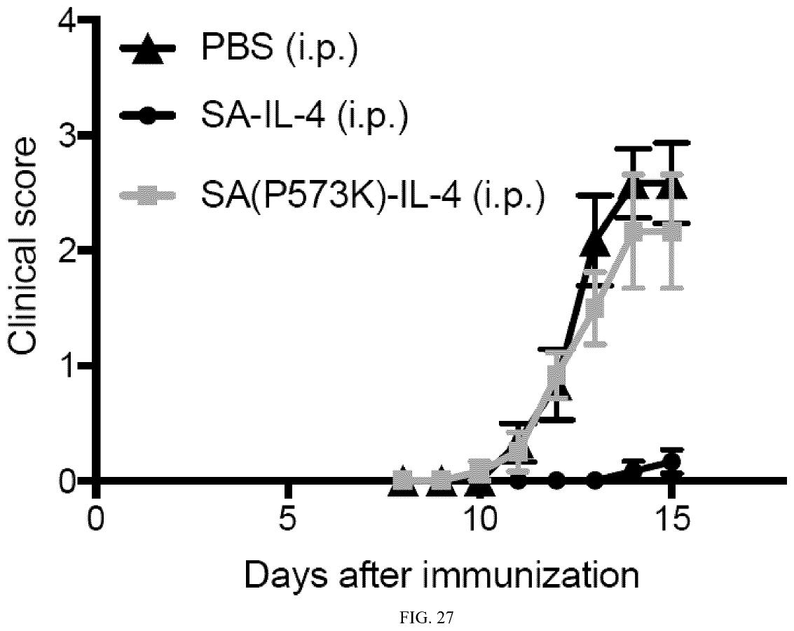

The inventors next treated myelin oligodendrocyte glycoprotein (MOG) antigen-induced EAE with SA-IL-4 in the acute phase of EAE (FIG. 20). SA-IL-4 was injected subcutaneously (s.c.) or intraperitoneally (i.p.). The inventors chose s.c. injection because it is clinically convenient and drug is absorbed slowly from the injection site. Injection i.p. was done as a surrogate for i.v. because the tail becomes flaccid in mice that have developed EAE, and tail vein injection thus becomes difficult. The inventors further compared the therapeutic effect of SA-IL-4 with FTY720, a clinically approved drug (fingolimod) for treating MS that sequesters lymphocytes in the LNs and prevents them from reacting with autoantigens in target tissues (3). Subcutaneous injection of SA-IL-4 completely suppressed disease development in all mice (FIG. 20A,B). SA-IL-4 injected i.p. and FTY720 prevented EAE development in 4 of 7 mice and suppressed disease severity in the rest. Wt IL-4 treatment did not show EAE clinical score suppression compared to the PBS treated group, all of which mice developed disease. Using body weight changes as a clinal indicator of health, mice treated with PBS and wt IL-4 were observed to markedly lose weight (FIG. 20C). Mice injected s.c. with SA-IL-4 gained their weight more than all other groups, indicating good health. The i.p. SA-IL-4 group gained weight on average, while FTY720 treated mice maintained their body weight. The inventors next analyzed demyelination of the spinal cord, which is the main morphological manifestation of EAE disease (FIG. 20D). Importantly, mice injected s.c. with SA-IL-4 did not have any detectable demyelination, demonstrating prevention of damage in the spinal cord. All wt IL-4-treated mice showed demyelination. The inventors then monitored mice long-term, until day 24, where SA-IL-4 i.p. injection inhibited disease development and progression (FIG. 26). Importantly, SA (P573K)-IL-4 did not suppress disease score (FIG. 27). These data suggest that SA fusion to IL-4 strongly improves the therapeutic effects of IL-4 on EAE disease suppression.

C. SA-IL-4 Treatment Suppresses Immune Cell Infiltration into the Spinal Cord and induces an immune-suppressive environment in the dLN

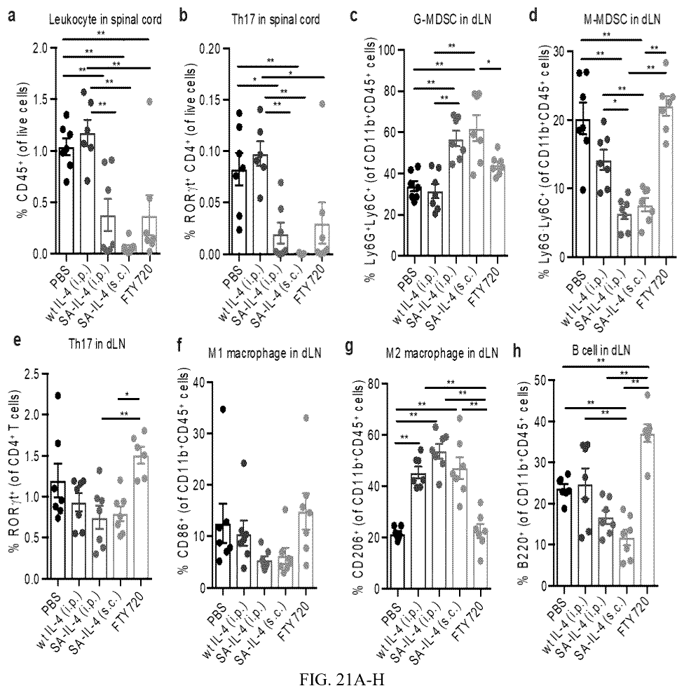

The inventors next analyzed immune cells in the spinal cord and dLN after treatment. Strikingly, s.c. injection of SA-IL-4 significantly suppressed immune cell infiltration into the CNS; very few CD45⁺immune cells were detected within the spinal cord (FIG. 21A). Hence, Th17 cells in the spinal cord were barely detectable in the s.c. SA-IL4 treatment group (FIG. 21B). I.p. injection of SA-IL-4 suppressed immune cell infiltration in 4 of 7 mice, which corresponds to the incidence of EAE disease in that group. FTY720 administration also suppressed immune cell infiltration into the spinal cord, as expected. Wt IL-4 did not show any effects on immune cell infiltration, including Th17, into the spinal cord compared to PBS treatment.

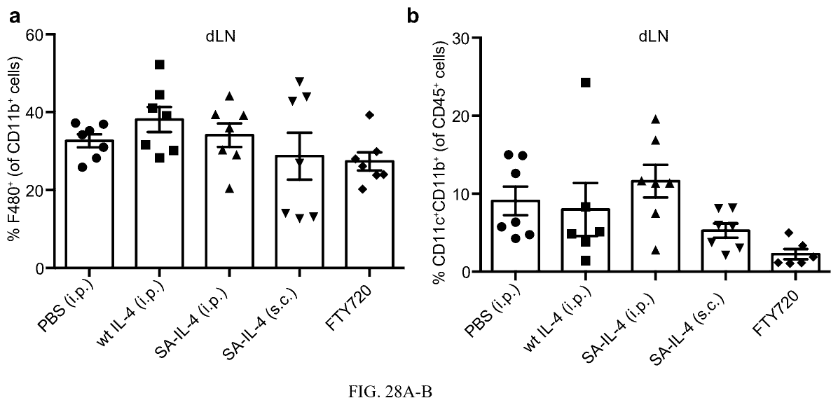

The inventors then analyzed immune cells in the lumbar dLN. SA-IL-4 increased granulocyte-like myeloid-derived suppressor cells (G-MDSCs), but reduced monocyte-like MDSCs (M-MDSCs) (FIG. 21C,D). The frequency of Th17 cells within CD4⁺ T cells in the dLN was also reduced by SA-IL-4 treatment (both i.p. and s.c.), compared to FTY720 treatment (FIG. 21E). FTY720 treatment trended to increase Th17 cell frequency in the dLN compared to PBS group, probably because FTY720 inhibits lymphocyte egress from LNs. SA-IL-4 treatment reduced the frequency of M1 macrophages and increased M2 macrophages in the dLN (FIG. 21F). Wt IL-4 did not decrease the frequency of M1 macrophages but increased M2 macrophages. The frequency of macrophages within CD11b⁺ cells was maintained (FIG. 28A), as well as the frequency of DCs within CD45⁺ cells (supplementary FIG. 4b). B cells reportedly promote induction of EAE by facilitating reactivation of T cells (14). SA-IL-4 (s.c.) decreased the frequency of B cells compared to both PBS and FTY720 treatment groups (FIG. 21H). Taken together, these data demonstrate that SA-IL-4 treatment generates an immunosuppressive environment in dLN and prevents immune cell infiltration into the spinal cord.

### D. SA-IL-4 Treatment Decreases IL-17-Related Cytokine and Integrin Expression On Antigen-Reacting CD4⁺T Cells

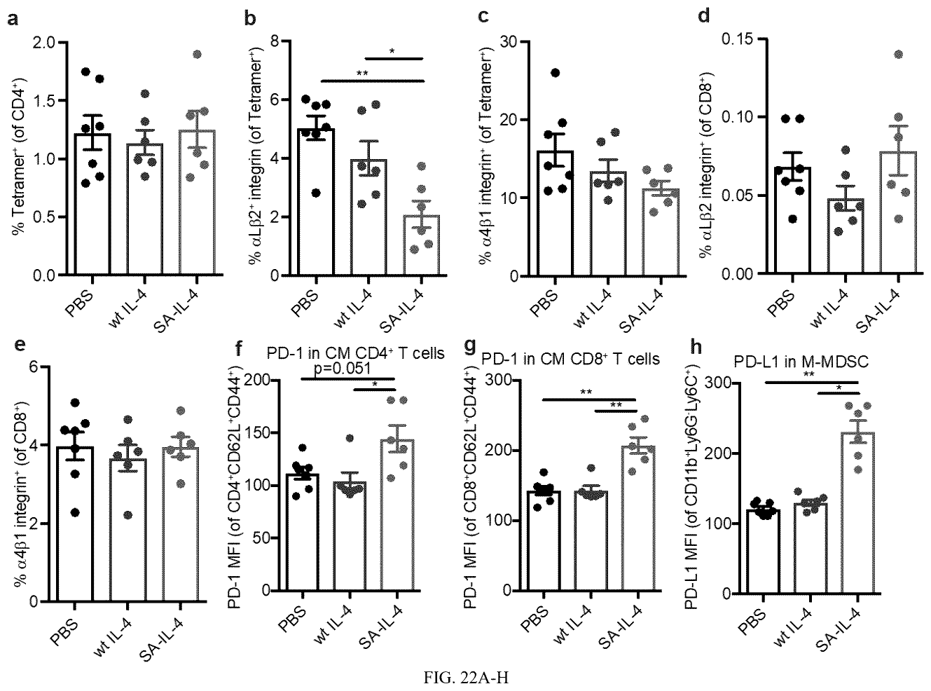

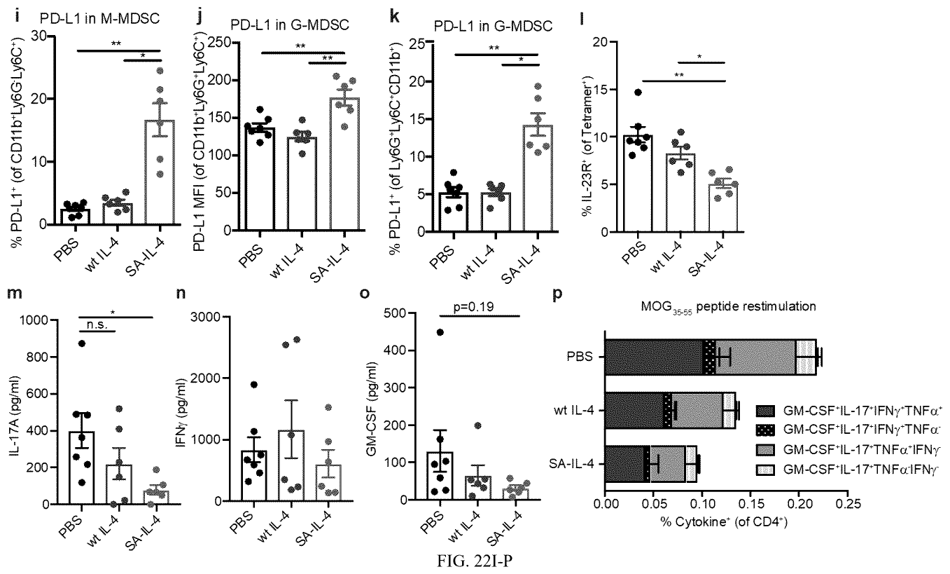

The inventors next analyzed the molecular mechanisms of decreased immune cell infiltration in the spinal cord and of the complete EAE disease prevention by s.c. injection of SA-IL-4. The inventors found that the number of MOG₃₅₋₅₅-reacting T cells in the dLN was maintained in all treatment groups, suggesting that SA-IL-4 does not change antigen recognition (FIG. 22A). Thus, the inventors hypothesized that SA-IL-4 changes T cell functionality. The inventors first tested the migratory ability of T cells. Expression levels of αLβ2 and α4B1 integrins, crucial adhesion molecules for lymphocytes migration (15), are reportedly decreased by IL-4 (16). The inventors found that SA-IL-4 treatment significantly decreased αLβ2 integrin expression on MOG₃₅₋₅₅-reacting CD4⁺T cells, but not on total CD8⁺ T cells (FIG. 22B-E). Expression of α₄β1 integrin was not significantly changed on MOG₃₅₋₅₅-reacting CD4⁺T cells, neither on total CD8⁺T cells. Because αLβ2 integrin is indispensable for Th17 cell infiltration into the spinal cord (15), this suggests that down-regulation of integrin expression is one of the mechanisms by which SA-IL-4 decreases lymphocyte migration to the spinal cord.

The inventors then tested PD-1 expression on T cells and PD-L1 expression on MDSCs (FIG. 22F-K), as PD-1 and PD-L1 association suppresses T cell activation (17). Strikingly, SA-IL-4 but not wt IL-4 increased the expression of PD-1 on both central memory CD4⁺ T cells and central memory CD8⁺ T cells (FIG. 22F,G). Moreover, SA-IL-4 but not wt IL-4 increased the expression levels of PD-L1 and the frequency of PD-L1-expressing cells on both M-MDSCs and G-MDSCs (FIG. 22H-K). These data suggest T cell suppression may be induced through MDSCs and the PD-1/PD-L1 axis.

The inventors next analyzed Th17-related protein expression. IL-23 is a crucial cytokine for Th17 functionality. IL-4 reportedly binds to APCs and silences IL-23 and concordant Th17 differentiation (18). The inventors found that SA-IL-4 treatment decreases the frequency of IL-23R⁺ cells within the MOG₃₅₋₅₅-reactive T cell repertoire (FIG. 22L).

Next, the inventors re-stimulated splenocytes with MOG protein (FIG. 22M,N). ELISA of culture supernatant revealed a decrease in IL-17A expression with SA-IL-4 treatment, but not wt IL-4 treatment, compared to PBS (FIG. 22M). The reduction in IL-17 expression implies a decreased number and/or level of activity of MOG₃₅₋₅₅-reactive Th17 cells in the SA-IL-4 treated group. The IFNγ concentration was maintained, suggesting little effect of SA-IL-4 on Th1 cells (FIG. 22N). SA-IL-4 trended toward a decreased level of GM-CSF, a reportedly pathogenic cytokine for EAE (19) (FIG. 220). The inventors then tested cytokine expression within T cells by flow cytometry after MOG₃₅₋₅₅ peptide re-stimulation of splenocytes (FIG. 22P). The inventors analyzed GM-CSF, IL-17, IFNγ and TNFα, all pathogenic cytokines for EAE. SA-IL-4 decreased the frequency of cytokine-expressing cells within the CD4⁺T cell compartment, compared to other treatments. These results strongly suggest that Th17 cells in the SA-IL-4-treated mice are fewer and less pathogenic compared to other treatment groups. Taken together, these data suggest that SA-IL-4 modulates multiple immune cell responses in the SLOs and suppresses EAE disease development through preventing immune cell infiltration, especially T cell infiltration, into the spinal cord.

### E. SA-IL-4 Recovers Paralysis Due to EAE in the Chronic Phase

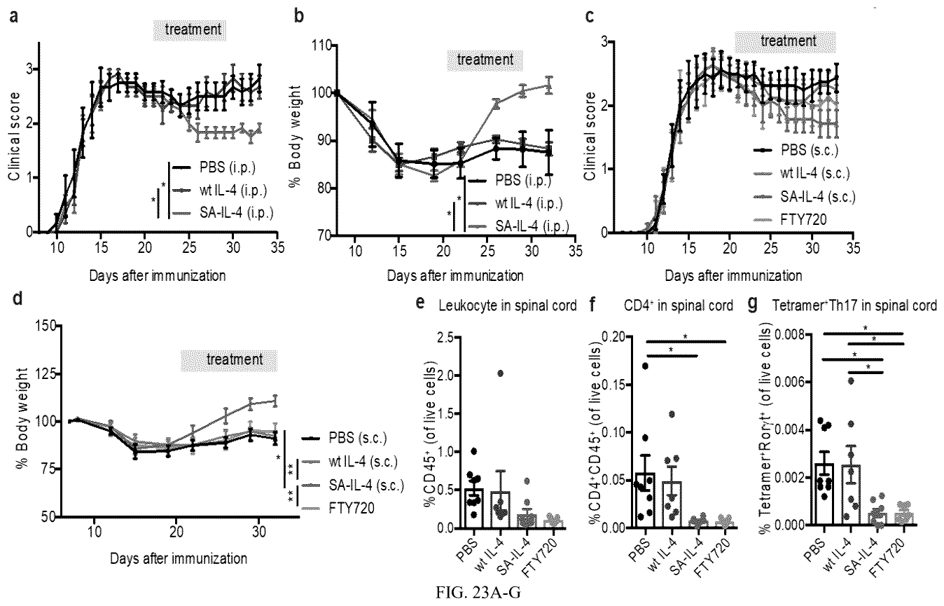

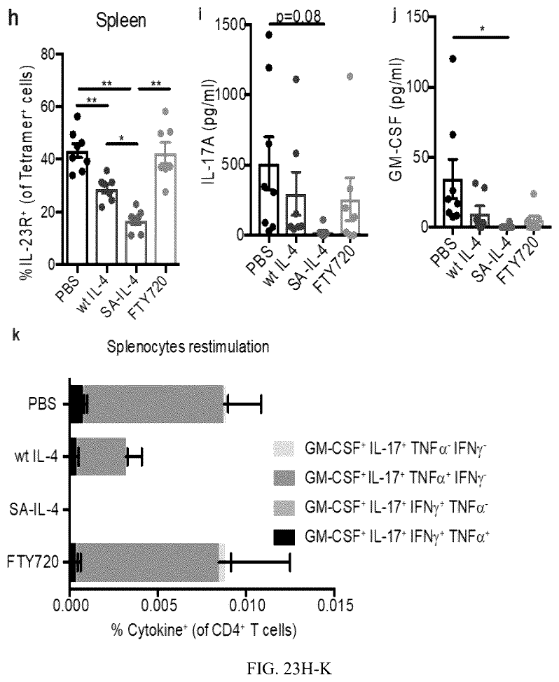

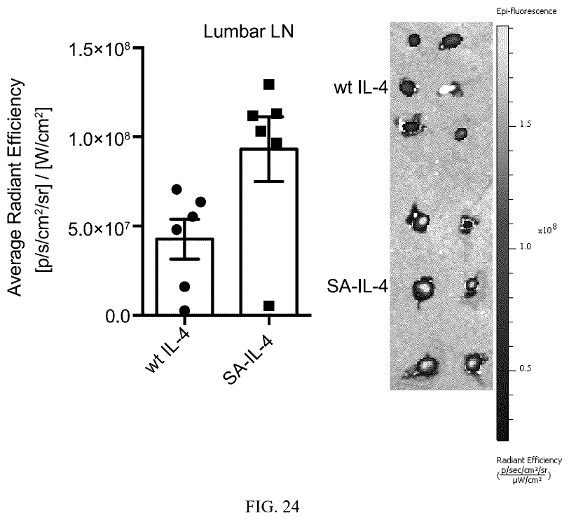

To determine if SA-IL-4 has a therapeutic effect in the chronic phase of EAE, the inventors designed an experiment involving treating mice that had already reached a stage of severe paralysis. The inventors began i.p. injection of IL-4 from day 21 after induction (FIG. 23A,B). Strikingly, SA-IL-4 but not wt IL-4 demonstrated therapeutic efficacy even when administered at this late time point. SA-IL-4-but not wt IL-4-treated mice gained weight, indicating disease recovery. The inventors next tested the effect of SA-IL-4 in the chronic phase via s.c. injection, and compared with oral FTY720 therapy (FIG. 23C,D). As a result, SA-IL-4-treated mice trended toward a decrease of the clinical score compared to other treatment groups. Mice receiving SA-IL-4 gained weight compared to other groups. FTY720- and wt IL-4-treated mice did not gain weight compared to PBS-treated mice.

The inventors then tested immune cell infiltration into the spinal cord at day 34 after induction by flow cytometry (FIG. 23E-G). SA-IL-4 and FTY720 treatment decreased the number of spinal cord infiltrated immune cells, including CD4⁺ T cells and MOG₃₅₋₅₅-reactive Th17 cells, compared to PBS and wt IL-4 treatment. SA-IL-4 decreased IL-23R expressing cells within MOG₃₅₋₅₅-reactive CD4⁺ T cells in the spleen compared to other treatment groups (FIG. 23H). Finally, splenocyte re-stimulation with MOG protein was performed. ELISA of the culture supernatant revealed decreased IL-17A and GM-CSF concentrations in the SA-IL-4 treatment group, but not after wt IL-4 and FTY720 treatment, compared to PBS (FIG. 23I,J). Flow cytometric analysis after MOG₃₅₋₅₅ peptide re-stimulation showed that SA-IL-4 decreased the frequency of cytokine-expressing cells within the CD4⁺T cell compartment compared to other treatments. (FIG. 23K). Taken together, these results suggest that SA-IL-4 treatment has a potent therapeutic effect on the EAE chronic phase.

F. SA-IL-4 Did not Show Marked Toxicity after Systemic Injection

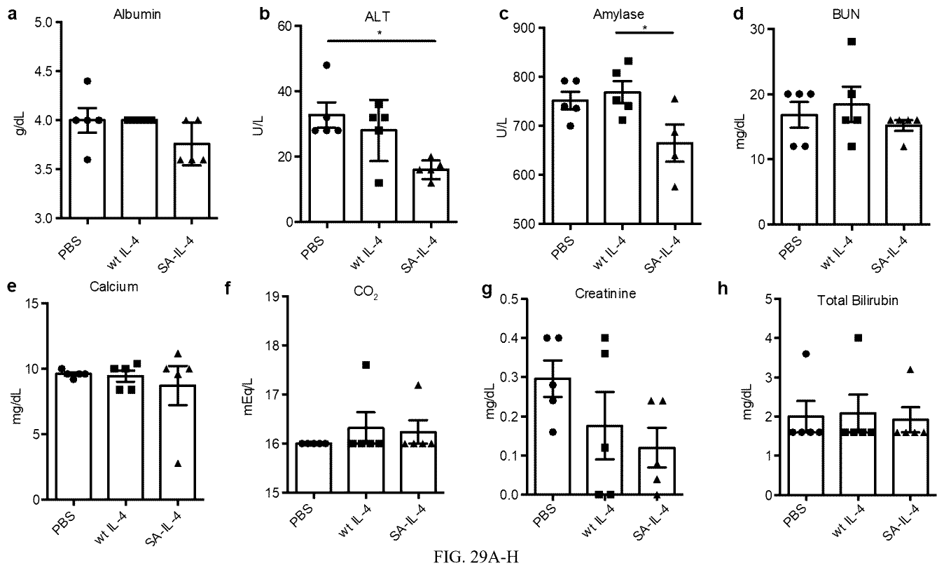

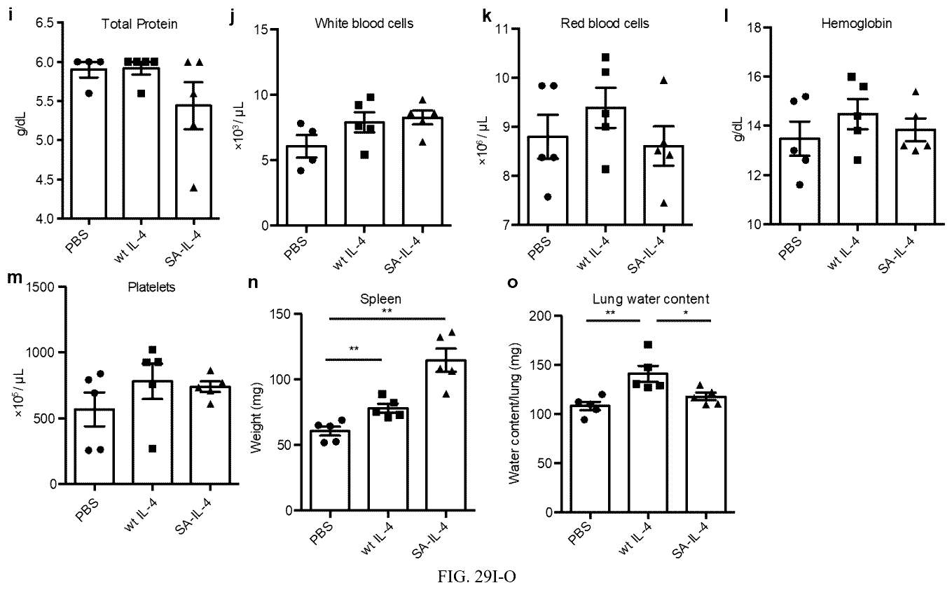

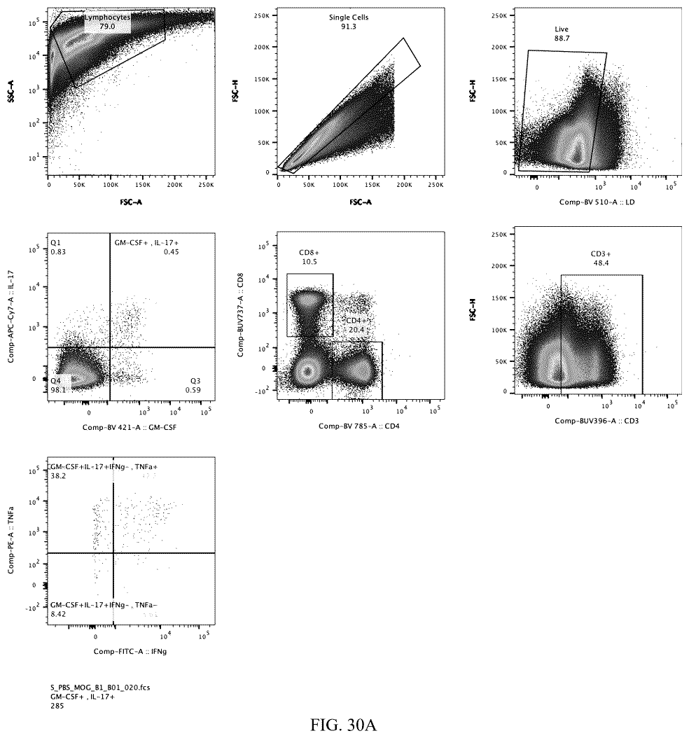

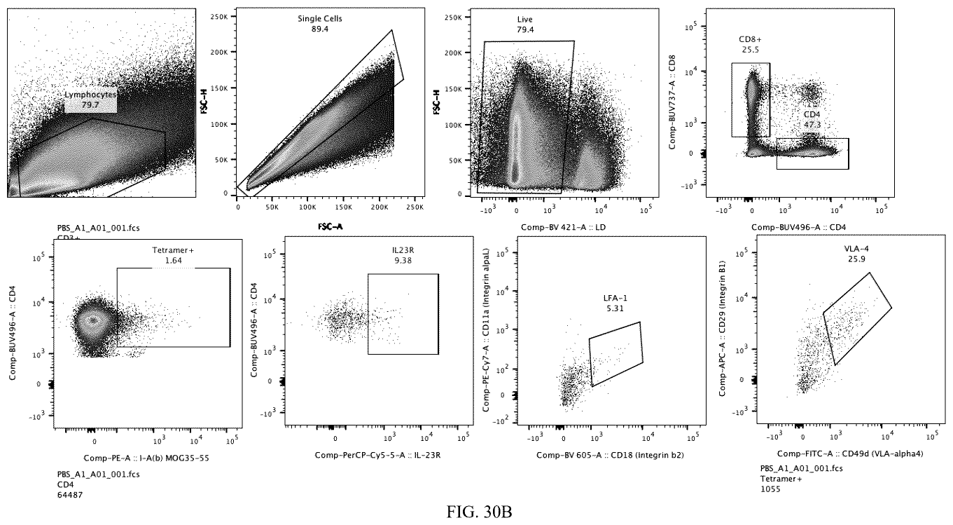

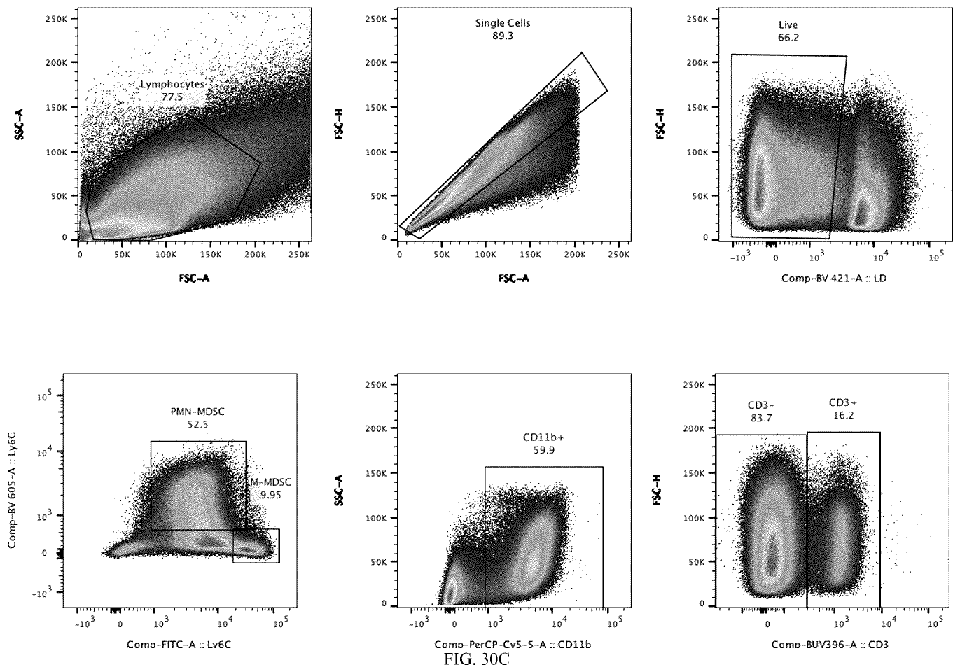

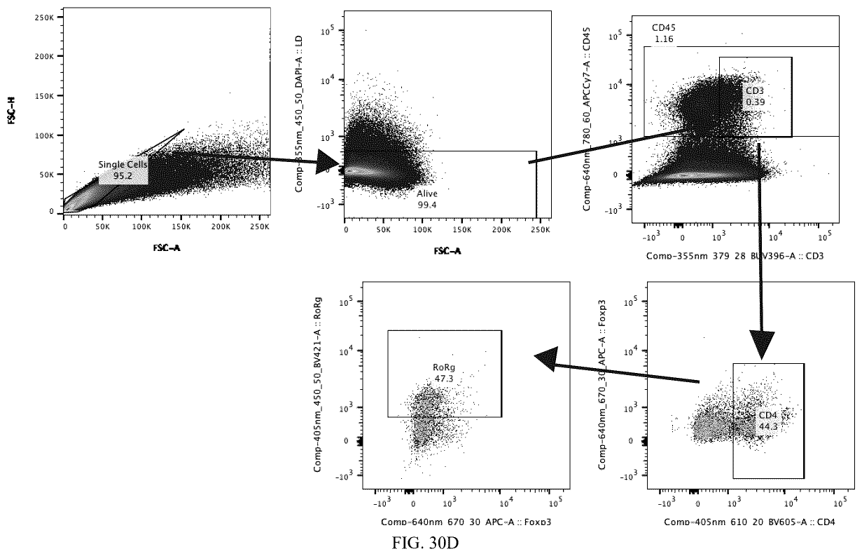

To test if SA-IL-4 exhibits any adverse effects, the inventors analyzed serum using a biochemistry analyzer and blood using a hematology analyzer (FIG. 29). SA-IL-4 treatment did not increase organ damage markers nor change blood cell counts (FIG. 29A-M). SA-IL-4 and wt IL-4 induced splenomegaly (FIG. 29N). Wt IL-4 induced pulmonary edema, indicated by water content increase in the lung, whereas SA-IL-4 did not (FIG. 29N). These evidence suggests that SA-IL-4 is safe after systemic administration.

Although a number of treatments for MS are currently available in the clinic, the disease remains poorly addressed. Some effective therapies, such as with FTY720 (fingolimod) and anti-α₄ integrin (natalizumab) inhibit effector lymphocyte infiltration into the diseased tissue, which is associated with risk of immune-related adverse events (20). IFNβ functions by multiple mechanisms, including reduction of lymphocyte migration into the CNS (21). Although IFNβ is not as effective as fingolimod (22), IFNβ is employed clinically to modulate pathology in MS (23). Here, the inventors seek to explore a therapy that could sculpt the immune response away from the Th17 pathway that is known to be involved in the pathology of the disease, toward a more tolerogenic phenotype using IL-4, without adverse effects. Here, the inventors utilized a molecular engineering approach to target IL-4 to the SLOs to ameliorate the underlying autoimmune response to myelin antigens.

In this study, s.c. injection of SA-IL-4 prevented the development of EAE in all mice tested. Strikingly, SA-IL-4 strongly reduced lymphocyte infiltration into the spinal cord with treatment at both early and late time points. Analysis of the dLN revealed that SA-IL-4 increased M2 macrophage and G-MDSCs, as well as decreased Th17 cells. However, wt IL-4 also increased the number of M2 macrophages, suggesting that the IL-4-mediated suppression of inflammatory macrophages is insufficient to control EAE under these experimental conditions.

IL-4 reportedly maintains and increases the immunosuppressive character of MDSCs (24). G-MDSCs are a crucial population that suppresses EAE development (17). G-MDSCs express PD-L1 to induce functional suppression of T cells. On the other hand, M-MDSCs were decreased by SA-IL-4 treatment. The role of M-MDSCs on EAE is controversial, and a pathogenic effect has been reported (25). Interestingly, SA-IL-4 but not wt IL-4 enhanced expression of PD-L1 on both G- and M-MDSCs in the SLOs. Simultaneously, SA-IL-4 but not wt IL-4 enhanced expression of PD-1 on both CD4⁺ and CD8⁺ central memory T cells in the SLOs. The inventors suppose that this PD-1/PD-L1 induction effect is the mechanism through which MDSCs in the SA-IL-4 treated mice suppress pathogenic T cells, and the observation of this phenomenon in the SLOs attests to the value of the SLO-targeting capability conferred by SA fusion.

Th17 cells play a crucial role in EAE disease severity and development (26). SA-IL-4 treatment reduced the frequency of Th17 cells in the dLN, compared to FTY720 treatment. IL-4 directly inhibits naïve T cell differentiation into Th17 cells, as the inventors have shown in FIG. 18D. Additionally, there is a possible Th17 inhibition pathway mediated through APCs. IL-23 is expressed by APCs (26) and generates pathogenic Th17 cells and induces expression of IL-17 (27). IL-4 reportedly reduces expression of IL-17 through silencing of IL-23 in APCs (18). Thus, it may be that SA-IL-4 acts on APCs to decrease IL-23 expression, thereby blocking generation of pathogenic Th17 cells in the SLOs. This model agrees with the inventors' data that SA-IL-4 decreases GM-CSF⁺-mediated T cell pathogenicity, as GM-CSF⁺is a marker for pathogenic Th17 cells.

Additionally, SA-IL-4 treatment resulted in decreased integrin expression in the MOG-reactive CD4⁺T cell compartment. Although immunized mice had already mounted an anti-MOG response before treatment, SA-IL-4 was able to prevent the migration of autoimmune cells into the CNS. Therefore SA-IL-4 treatment did not change the number of antigen-reacting T cells, but rather inactivated T cell function. Overall, this data show that SA-IL-4 acts through multiple immune pathways to generate an immunosuppressive environment in the SLOs.

FTY720 is one of the FDA-approved drugs against MS, functioning through induction of lymphocytopenia and thus limiting migration of effector lymphocytes to the CNS disease site. Subcutaneously administered SA-IL-4 showed higher efficacy than FTY720 in this study. This improvement with SA-IL-4 is strong evidence for its potential for clinical translation. FTY720 has the drawback that it cannot be administered to infected patients. Also, among the approved therapies for MS that modulate lymphocyte migration, such as FTY720 and integrin α₄ blockade, induction of John Cunningham virus (JCV) activity can lead to progressive multifocal leukoencephalopathy (PML) which can ultimately cause severe adverse events including death. SA-IL-4 decreased integrin expression only on the antigen-reactive CD4⁺T cells, but not CD8⁺T cells. This suggests that SA-IL-4 has additional migratory inhibitory effects on antigen-reactive CD4⁺T cells, which may contribute to inflammation suppression in the spinal cord. The hematology study showed that SA-IL-4 did not induce lymphocytopenia. This may be an additional advantage over approved drugs, as SA-IL-4 is not expected to strongly suppress the immune response against infectious diseases, and thus it may be that SA-IL-4 may be useful for patients who are not amenable to treatment with FTY720. SA-IL-4 likely suppresses autoimmunity through various mechanisms that are not redundant with current therapeutics. In this study, FTY720 did not suppress Th17 development in the dLN, while SA-IL-4 did. Thus, SA-IL-4 may be useful for patients who do not obtain sufficient therapeutic effects with current treatments.

Intradermal or s.c. injections of SA have been well studied to accumulate in the injection site-draining LN (28). Here, the inventors showed that SA-IL-4 accumulates in the LNs after i.v. injection, i.e. from the blood rather than via the afferent lymphatic vessel. Although previously a biodistribution study of SA-fused IL-2 showed spleen, liver and LN accumulation of IL-2 through i.v. injection (29), the molecular mechanism and its localization within LNs were unclear. The histological analysis shows that i.v. injected SA-IL-4 accumulates and co-localizes around high endothelial venules (HEVs) (30). Although IgG transcytosis through FcRn has been well studied, only recently has the transcytosis of albumin through FcRn binding been reported by multiple research groups (31-33). Because FcRn is highly expressed in the LN (34), it is likely that i.v.-injected SA-IL-4 trafficked to the LN by transcytosis. In this study, SA (P573K)-IL-4 achieved lower amounts of IL-4 in the LN compared to SA-IL-4, similar to wt IL-4. This would suggest that FcRn binding plays an important role in SA-IL-4 transport to the LN from the blood. From the inventors' observation of SA-IL-4 concentrating around the medullary space, where APCs reside, the inventors postulate that SA-IL-4 enters through HEVs and then binds to DCs and macrophages, as the inventors have shown in FIG. 18. SA (P573K)-IL-4 did not suppress disease score in FIG. 27, which indicates that FcRn binding and subsequent increased persistence in the SLO are crucial to suppress EAE disease symptoms. Thus, the inventors believe that the SA-cytokine fusion immunosuppressive molecules function not primarily by extending blood circulation time, rather by extending the recycle time in the SLO, such as the LNs and spleen. Thus, the SA fused cytokine can be collected by one immune cell and recycled by that cell to stimulate another immune cell. As such, blood circulation lifetimes are not directly related to SLO presence lifetimes. The inventors believe that this biological finding can open a new research field of LN molecular trafficking, and further research will clarify the detailed mechanisms.

SA-IL-4 exhibited therapeutic efficacy in the chronic phase of EAE. Nasal or lumbar administration of IL-4 has been previously reported to lead to direct binding of IL-4 to neurons, in attempts to regenerate the nervous system in EAE (9). The therapeutic effect of i.p.-injected SA-IL-4 was comparable to nasally administered wt IL-4 in the previous report. This suggests that SA-IL-4 may also bind to neurons and induce regeneration. Because the blood brain barrier is disrupted in the EAE model, SA-IL-4 may have access to neurons within the spinal cord. It will be interesting to study the direct effect of SA-IL-4 on neural regeneration in future research.

Subcutaneous administration of SA-IL-4 showed higher preventative effect than that of i.p. injection. This route is convenient for clinical translation and generally shows slow release from the tissue. The inventors assume that this slow release was a further contributing factor for suppression of EAE development.

Systemically injected SA-IL-4 did not show obvious toxicity in the blood biochemistry analysis and hematology analysis. Although the inventors have observed splenomegaly, this is typically transient and is not considered as a critical toxicity. Wt IL-4 induced pulmonary edema, which would be more concerning. However, pulmonary edema was not observed with SA-IL-4; this difference may be because of diminished activity of SA-IL-4 on STAT6 activation. These results indicate a low risk of adverse event induction by SA-IL-4 administration in the clinic.

In conclusion, the engineered SA-IL-4 demonstrated persistence in SLOs through FcRn binding. SA-IL-4 displayed marked therapeutic effects on multiple phases of EAE after systemic injection, and it modulates key immune pathways of EAE, such as decreasing Th17 cells and increasing G-MDSCs and PD-L1 expression by MDSCs in SLOs. SA-IL-4 holds translational potential in both preventative and therapeutic usage through a novel biologic approach utilizing mechanisms that are different from currently approved therapies.

G. Amino acid sequence of wt iL-4 and SA-IL-4

| | |
|---|---|
| Mouse IL- 4 | MHIHGCDKNHLREIIGILNEVTGEGTPCTEMDVPNVLTATKNTTE |
| | SELVCRASKVLRIFYLKHGKTPCLKKNSSVLMELQRLFRAFRCLD |
| | SSISCTMNESKSTSLKDFLESLKSIMQMDYSHHHHHH |
| | (SEQ ID NO: 56) |
| Mouse serum | EAHKSEIAHRYNDLGEQHFKGLVLIAFSQYLQKCSYDEHAKLVQ |
| albumin and | EVTDFAKTCVADESAANCDKSLHTLFGDKLCAIPNLRENYGELA |
| mouse IL-4 | DCCTKQEPERNECFLQHKDDNPSLPPFERPEAEAMCTSFKENPTT |
| fusion protein | FMGHYLHEVARRHPYFYAPELLYYAEQYNEILTQCCAEADKESC |
| | LTPKLDGVKEKALVSSVRQRMKCSSMQKFGERAFKAWAVARLS |
| | QTFPNADFAEITKLATDLTKVNKECCHGDLLECADDRAELAKYM |
| | CENQATISSKLQTCCDKPLLKKAHCLSEVEHDTMPADLPAIAADF |
| | VEDQEVCKNYAEAKDVFLGTFLYEYSRRHPDYSVSLLLRLAKKY |
| | EATLEKCCAEANPPACYGTVLAEFQPLVEEPKNLVKTNCDLYEK |
| | LGEYGFQNAILVRYTQKAPQVSTPTLVEAARNLGRVGTKCCTLPE |
| | DQRLPCVEDYLSAILNRVCLLHEKTPVSEHVTKCCSGSLVERRPC |
| | FSALTVDETYVPKEFKAETFTFHSDICTLPEKEKQIKKQTALAELV |
| | KHKPKATAEQLKTVMDDFAQFLDTCCKAADKDTCFSTEGPNLVT |
| | RCKDALAGGGGSGGGSMHIHGCDKNHLREIIGILNEVTGEGTPCT |
| | EMDVPNVLTATKNTTESELVCRASKVLRIFYLKHGKTPCLKKNSS |
| | VLMELQRLFRAFRCLDSSISCTMNESKSTSLKDFLESLKSIMQMD |
| | YSHHHHHH (SEQ ID NO: 57) |

### H. Materials and Methods

### 1. Production and Purification of Recombinant Proteins

The sequences encoding for mouse SA without pro-peptide (25 to 608 amino acids of whole serum albumin), mouse IL-4, and a (GGGS) 2 linker were synthesized and subcloned into the mammalian expression vector pcDNA3.1 (+) by Genscript. A sequence encoding for 6 His was added at the C-terminus for further purification of the recombinant protein. The amino acid sequence of the protein is shown in the supplemental table 1. Suspension-adapted HEK-293F cells were routinely maintained in serum-free FreeStyle 293 Expression Medium (Gibco). On the day of transfection, cells were inoculated into fresh medium at a density of 1×10⁶ cells/ml. 2 μg/ml plasmid DNA, 2 μg/ml linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO₂. Seven days after transfection, the cell culture medium was collected by centrifugation and filtered through a 0.22 μm filter. Culture media was loaded into a HisTrap HP 5 ml column (GE Healthcare), using an ÄKTA pure 25 (GE Healthcare). After washing the column with wash buffer (20 mM NaH₂PO₄, 0.5 M NaCl, pH 8.0), protein was eluted with a gradient of 500 mM imidazole (in 20 mM NaH₂PO₄, 0.5 M NaCl, pH 8.0). The protein was further purified by size exclusion chromatography using a HiLoad Superdex 200PG column (GE Healthcare) using PBS as an eluent. All purification steps were carried out at 4° C. The expressed proteins were verified as >90% pure by SDS-PAGE. Purified proteins were tested for endotoxin via the HEK-Blue TLR4 reporter cell line, and endotoxin levels were confirmed to be less than 0.01 EU/ml. Protein concentration was determined through absorbance at 280 nm using NanoDrop (Thermo Scientific).

### 2. Mice

C57BL/6 female mice at 8 weeks of age mice were obtained from the Charles River Laboratories. Mice were housed at the University of Chicago Animal facility for at least 1 week before immunization. All experiments were performed with approval from the Institutional Animal Care and Use Committee of the University of Chicago.

### 3. Binding of the Proteins to Splenocytes or LN-Derived Cells

Single-cell suspensions were obtained by gently disrupting the spleen or popliteal lymph node through a 70-μm cell strainer. Red blood cells were lysed with ACK lysing buffer (Quality Biological) for splenocytes. Cells were counted and re-suspended in RPMI-1640 supplemented with 10% FBS and 1% penicillin/streptomycin (all from Life Technologies). 1×10⁵ cells/well were seeded in a 96 well microplate and were incubated with 2 μg/100 μl of SA, SA-IL4 for 30 min on ice. After 4-times washing by PBS, cells were further incubated with Rabbit monoclonal anti-mouse serum albumin antibody (clone EPR20195 abcam) for 20 min on ice. After 3-times washing by PBS, cells were incubated with 1 μg/ml AlexaFluor 647-labeled anti-Rabbit IgG, anti-B220, anti-CD3, anti-CD4, anti-CD8, anti-CD11c, anti-CD45 and anti-F4/80 antibodies for 20 min on ice. Cells were analyzed by flow cytometry as described below.

### 4. Analysis of STAT6 Phosphorylation by Flow Cytometry

Mouse CD4⁺ T cells were purified from spleens of C57BL/6 mice using EasySep mouse CD4⁺T cell isolation kit (Stem Cell). Purified CD4⁺T cells (10⁶ cells/ml) were activated in six-well plates precoated with 5 μg/ml anti-CD3 antibody (clone 17A2, Bioxcell) and supplemented with soluble 2 μg/ml anti-CD28 antibody (clone 37.51, BioLegend) for 2 days. Culture medium was IMDM (Gibco) containing 10% heat-inactivated FBS, 1% Penicillin/Streptomycin and 50 μM 2-mercaptoethanol (Sigma Aldrich). After 2 days of culture, activated CD4⁺ T cells were stimulated with 50 ng/ml recombinant murine IL-2 (Peprotech) for 3 hr to induce IL-4Rα expression. After stimulation with IL-2, cells were washed and rested in fresh medium for 3 hr. Cells were then transferred into 96-well plates (50,000 cells/well). Indicated amounts of wt IL-4 or SA-IL-4 were applied to CD4⁺T cells for 15 min at 37° C. to induce STAT6 phosphorylation. Cells were fixed immediately using BD Phosflow Lyse/Fix buffer for 10 min at 37° C. and then permeabilized with BD Phosflow Perm Buffer III for 30 min on ice. Cells were stained with AlexaFluor 647 anti-pSTAT6 antibody (Clone J71-773.58.11, BD) recognizing phosphorylation of Tyr641. Staining was performed for 1 hr at room temperature (RT) in the dark. Cells were acquired on BD LSR and data were analyzed using FlowJo (Treestar). Mean fluorescence intensity (MFI) of pSTAT6⁺ population was plotted against cytokine concentration. Dose-response curve was fitted using Prism (v8, GraphPad).

### 5. Surface Plasmon Resonance (SPR)

SPR measurements were made with a Biacore X100 SPR system (GE Healthcare). Murine FcRn recombinant protein (Acro Biosystems) was immobilized via amine coupling on a C1 chip (GE Healthcare) for ˜200 resonance units (RU) according to the manufacturer's instructions. SA-IL4 was flowed at decreasing concentrations in the running buffer (0.01 M monobasic anhydrous sodium phosphate, pH 5.8, 0.15 M NaCl) at 30 μl/min. The sensor chip was regenerated with PBS, pH 7.4 for every cycle. Specific bindings of SA fusion proteins to FcRn were calculated by comparison to a non-functionalized channel used as a reference. Experimental results were fitted with Langmuir binding kinetics using BIAevaluation software (GE Healthcare).

### 6. Th17 Cell Differentiation In Vitro in Culture

Naïve CD4⁺ T cells were isolated from splenocytes using EasySep™ Mouse Naïve CD4⁺T Cell Isolation Kit (STEMCELL Technologies) according to the manufacture's instructions. 10⁵ cells were plated in the 96 well plate and cultured for 3 days. As Th17 induction media,

IL-17A concentrations in the culture media were measured by IL-17 Ready-Set-Go! Mouse Uncoated ELISA kit (Invitrogen) according to the manufacturer's protocol. Data were analyzed using Prism software (v6, GraphPad).

### 7. Plasma Pharmacokinetics of the Proteins

Wt IL-4 or SA-IL-4 (equivalent to 10 μg of IL-4) was injected intravenously into female C57BL/6 mice. Blood samples were collected in protein-low binding tubes at 1 min, 10 min, 30 min, 1 hr, 2 hr, 4 hr and 24 hr after injection. IL-4 concentrations in plasma were measured by IL-4 Ready-Set-Go! Mouse Uncoated ELISA kit (Invitrogen) according to the manufacturer's protocol.

### 8. Lymph Node and Spleen Pharmacokinetics of the Proteins

Wt IL-4, SA-IL-4 or SA (P573K)-IL-4 (equivalent to 40 μg of IL-4) was injected intravenously into healthy C57BL/6 mice. Lumbar and brachial LNs and spleen were collected at 1 hr, 4 hr, and 24 hr after injection, and were subsequently homogenized using Lysing Matrix D and FastPrep-24 5G (MP Biomedical) for 40 s at 5000 beats/min in T-PER tissue protein extraction reagent (Thermo Scientific) with complete™ proteinase inhibitor cocktail (Roche). After homogenization, samples were incubated overnight at 4° C. Samples were centrifuged (5000 g, 5 min), and the total protein concentration and IL-4 concentration were analyzed using a BCA assay kit (Thermo Fisher) and IL-4 Mouse Uncoated ELISA kit (Invitrogen), respectively. Simultaneously, cytokine levels in LN extract were measured using Mouse Uncoated ELISA kit (Invitrogen) or Ready-SET-Go! ELISA kits (eBioscience) according to the manufacturer's protocol.

### 9. Fluorescence Based IL-4 Detection in the LN

To make fluorescently labeled wt IL-4 and SA-IL-4, proteins were incubated with 8-fold molar excess of using DyLight 800 NHS ester (Thermo Fisher) for 1 hr at RT, and unreacted dye was removed by a Zebaspin spin column (Thermo Fisher) according to the manufacturer's instruction. 10 μg of DyLight 800-labeled wt IL-4 and SA-IL-4 with equivalent fluorescence were injected i.v. to naïve C57BL/6 mice. After 4 hr, the iliac LN was imaged with the Xenogen IVIS Imaging System 100 (Xenogen) under the following conditions: f/stop: 2; optical filter excitation 745 nm; excitation 800 nm; exposure time: 5 sec; small binning.

### 10. Immunofluorescence

Wt IL-4 and SA-IL-4 were fluorescently labeled with DyLight 594 NHS ester (Thermo Fisher), as described above. 1 hr after i.v. injection of fluorescently-labeled IL-4 (40 μg for wt IL-4 and same fluorescent amount for SA-IL-4), mice were sacrificed. Mouse LNs were harvested and fixed in 2% PFA in PBS overnight and washed with PBS. After overnight incubations in 30% sucrose solutions, LNs were embedded in Optimum Cutting Temperature compound. Then, 5 μm cryosections were cut using a cryostat. Sections were then blocked with 2% BSA in PBS at RT and incubated with the following primary antibodies for 2 hr at RT: 10 μg/ml hamster anti-mouse CD3& antibody (clone: 145-2C11, BioLegend) and 2.5 μg/ml rat anti-mouse PNAd (clone: MECA-79, BioLegend) antibody. After washing with PBS-T, tissues were stained for 1 hr at RT with the following fluorescently-labeled secondary antibodies were used: Alexa Fluor 647 goat anti-hamster (1:400, Jackson ImmunoResearch) and Alexa Fluor 488 donkey anti-rat (1:400, Jackson ImmunoResearch). The tissues were washed three times and then covered with ProLong gold antifade mountant with 4′,6-diamidino-2-phenylindole (DAPI; Thermo Fisher Scientific). An IX83 microscope (Olympus) was used for imaging with 10× magnification for CD3 staining, and a Leica SP8 3D Laser Scanning Confocal microscope with 20× magnification for PNAd staining. Images were processed using ImageJ software (NIH).

### 11. EAE Model

C57BL/6 young female mice aged 9 to 12 wk were immunized subcutaneously at the dorsal flanks with an emulsion of MOG₃₅₋₅₅ in complete Freund's adjuvant (CFA), followed by intraperitoneal administration of pertussis toxin (PTX) in PBS, first on the day of immunization and then again the following day. MOG₃₅₋₅₅/CFA Emulsion and PTX were purchased from Hooke Laboratories. Following the first immunization, the severity of EAE was monitored and clinical scores were measured daily from day 8 after immunization. The clinical scores were determined by A.I., M.N. or A.S. based on the Hooke Laboratories criteria under blinding to the treatment grouping. IL-4, SA-IL-4, PBS was administered i.p. or s.c. (in the mouse back) in 100 μl PBS, every other day. FTY720 (1 mg/kg body weight) was administered orally every day.

### 12. Histology of Spinal Cord

The thoracic and lumbar spines of EAE mice were harvested and cut out at the thoracolumbar junction. Tissues were fixed in 2% PFA overnight. After PBS wash, tissues were decalcified using Decalcifier II (Leica Biosystem) overnight. Then, tissues were embedded in paraffin. After paraffin embedding, blocks were cut into 5 mm sections. After deparaffinization and rehydration, tissue sections were treated with target retrieval solution (S1699, DAKO) and heated in a steamer for 20 min at temperature>95 C°. Tissue sections were incubated with anti-mouse aMBP, abcam ab40390) for 1 hr incubation at RT in a humidity chamber. Following a TBS wash, the tissue sections were incubated with biotinylated anti-rat IgG (10 mg/mL, Vector laboratories) for 30 min at RT. The antigen-antibody binding was detected by Elite kit (PK-6100, Vector Laboratories) and DAB (DAKO, K3468) system. Slides were imaged by EVOS FL Auto (Life Technologies).

### 13. Flow Cytometry

EAE mice were treated with PBS, wt IL-4, or SA-IL-4 (equivalent to 10 μg of IL-4) every other day, starting 8 days after immunization. Thirteen, 17 or 34 days after immunization, the spinal cord, spleen, and lumbar LNs were harvested. Spinal cord tissues were digested in Dulbecco's Modified Eagle Medium (DMEM) supplemented with 2% FBS, 2 mg/ml collagenase D (Sigma-Aldrich) and 40 μg/ml DNase I (Roche) for 30 min at 37° C. Single-cell suspensions were obtained by gently disrupting through a 70-μm cell strainer. For spleen, red blood cells in blood were lysed with ACK lysing buffer (Quality Biological), followed by antibody staining for flow cytometry. Antibodies against the following molecules were used: anti-mouse CD38 (145-2C11, BD Biosciences), CD4 (RM4-5, BD Biosciences), anti-mouse CD8x (53-6.7, BD Biosciences), anti-mouse CD45 (30-F11, BD Biosciences), CD44 (IM7, BD Biosciences), CD62L (MEL-14, BD Biosciences), F4/80 (T45-2342, BD Biosciences), CD86 (GL1, BD Biosciences), CD206 (C068C2, BioLegend), Ly6G (1A8, BioLegend), Ly6C (HK1.4, BioLegend), CD11b (M1/70, BioLegend), CD11c (HL3, BD Biosciences), B220 (RA3-6B2, BioLegend), PD-1 (29F.1A12, BD Biosciences), PD-L1 (MIH7, BioLegend), IL-23R (078-1208, BD Biosciences), Integrin integrin αL (HI111, BD Biosciences), integrin β2 (M18/2, BD Biosciences), integrin β1 (HMb1-1, BD Biosciences), integrin α₄ (R1-2, BD Biosciences), GM-CSF (MP1-22E9, BD Biosciences), IL-17 (TC11-18H10.1, BD Biosciences), IFNγ (XMG1.2, BD Biosciences), TNFα (eBioscience, MP6-XT22), and RoRγt antibody (Q31-378, BD Biosciences). For detection of MOG-recognizing T cells, T-Select I-Ab MOG₃₅₋₅₅ Tetramer-PE (MBL International Corporation) or MOG₃₈₋₄₉ Tetramer-PE (NIH Tetramer Core Facility) was used. Fixable live/dead cell discrimination was performed using Fixable Viability Dye eFluor 455 (eBioscience), Live/Dead Fixable Violet (eBioscience), or Live/Dead Fixable Aqua (eBioscience), according to the manufacturer's instructions. Staining was carried out on ice for 20 min. For intracellular staining, Cytofix/Cytoperm (BD Bioscience) was used to fix cells for 20 min at 4° C. . . . For permeabilization, perm/wash buffer (BD Bioscience) was used, and cells were stained in perm/wash buffer for 30 min at 4° C. Following a washing step, cells were stained with specific antibodies for 20 min on ice prior to fixation. All flow cytometric analyses were done using a Fortessa (BD Biosciences) flow cytometer and analyzed using FlowJo software (Tree Star).

### 14. Re-Stimulation of Splenocytes

Single cell suspensions were created from the dLNs and spleen. For analysis of cytokine production, 5×10⁵ lymphocytes and 2×10⁶ splenocytes were plated in a 96 well round bottom plate. Cells were stimulated with 10 μM MOG₃₅₋₅₅ peptide (Genscript). After 2 hr, GolgiPlug (brefeldin A) and GolgiStop (Monensin) were added per manufacturer's protocol to block secretion of intracellular cytokines. Four hours after addition of GolgiPlug and GolgiStop, cells were stained for flow cytometry. For fixation, Cytofix/Cytoperm (BD Bioscience) was used for 20 min at 4° C. . . . For permeabilization, perm/wash buffer (BD Bioscience) was used, and cells were stained in perm/wash buffer for 30 min at 4° C. For 3 day restimulation, 2.5×10⁵ lymphocytes or 1×10⁶ splenocytes were plated in a 96 well round bottom plate. Cells were stimulated with 10 μM MOG₃₅₋₅₅ (for 6 hr culture followed by flow cytometry) or 100 μg/ml MOG protein (for 72 hr culture) (Anaspec). After 72 hr, supernatant was collected for analysis by ELISA using Ready-Set-Go! Kit (Invitrogen) or LEGEND MAX mouse GM-CSF ELISA kit (BioLegend).

### 15. Safety Assessments of SA-IL-4

C57BL/6 mice were intravenously injected with PBS, wt IL-4, or SA-IL4 (equivalent to 10 μg of IL-4). Two days after, blood samples collected from mice were analyzed using a COULTER Ac·T 5diff CP hematology analyzer (Beckman Coulter) according to the manufacturer's instructions. Lung and spleen were harvested and weighed. Water content in the lung was determined by weighing before and after overnight lyophilization using a FreeZone 6 Benchtop Freeze Dryer (Labconco). Serum samples collected from PBS, wt IL-4, and SA-IL-4-injected mice were analyzed using a Biochemistry Analyzer (Alfa Wassermann Diagnostic Technologies) according to the manufacturer's instructions.

### 16. Statistical Analysis

Statistically significant differences between experimental groups were determined using Prism software (v6, GraphPad). Where one-way ANOVA followed by Tukey's HSD post hoc test was used, variance between groups was found to be similar by the Brown-Forsythe test. For single comparisons, a two-tailed Student's t-test was used. The symbols * and ** indicate P values less than 0.05 and 0.01 respectively.

### I. REFERENCES

1. Friese, M. A., Schattling, B. & Fugger, L. Mechanisms of neurodegeneration and axonal dysfunction in multiple sclerosis. Nature reviews. Neurology 10, 225-238 (2014).
2. Ellwardt, E., Walsh, J. T., Kipnis, J. & Zipp, F. Understanding the Role of T Cells in CNS Homeostasis. Trends in immunology 37, 154-165 (2016).
3. Chun, J. & Hartung, H. P. Mechanism of action of oral fingolimod (FTY720) in multiple sclerosis. Clinical neuropharmacology 33, 91-101 (2010).
4. Rice, G. P., Hartung, H. P. & Calabresi, P. A. Anti-alpha4 integrin therapy for multiple sclerosis: mechanisms and rationale. Neurology 64, 1336-1342 (2005).
5. Cooney, L. A., Towery, K., Endres, J. & Fox, D. A. Sensitivity and resistance to regulation by IL-4 during Th17 maturation. Journal of immunology (Baltimore, Md.: 1950) 187, 4440-4450 (2011).
6. Gadani, S. P., Cronk, J. C., Norris, G. T. & Kipnis, J. IL-4 in the brain: a cytokine to remember. Journal of immunology (Baltimore, Md.: 1950) 189, 4213-4219 (2012).
7. Racke, M. K. et al. Cytokine-induced immune deviation as a therapy for inflammatory autoimmune disease. The Journal of experimental medicine 180, 1961-1966 (1994).
8. Butti, E. et al. IL4 gene delivery to the CNS recruits regulatory T cells and induces clinical recovery in mouse models of multiple sclerosis. Gene therapy 15, 504-515 (2008).
9. Vogelaar, C. F. et al. Fast direct neuronal signaling via the IL-4 receptor as therapeutic target in neuroinflammation. Science translational medicine 10 (2018).
10. van Zwam, M. et al. Surgical excision of CNS-draining lymph nodes reduces relapse severity in chronic-relapsing experimental autoimmune encephalomyelitis. The Journal of pathology 217, 543-551 (2009).
11. Dennis, M. S. et al. Albumin binding as a general strategy for improving the pharmacokinetics of proteins. The Journal of biological chemistry 277, 35035-35043 (2002).
12. Liao, W. et al. Priming for T helper type 2 differentiation by interleukin 2-mediated induction of interleukin 4 receptor alpha-chain expression. Nature immunology 9, 1288-1296 (2008).
13. Nilsen, J. et al. Human and mouse albumin bind their respective neonatal Fc receptors differently. Sci Rep 8, 14648-14648 (2018).
14. Pierson, E. R., Stromnes, I. M. & Goverman, J. M. B cells promote induction of experimental autoimmune encephalomyelitis by facilitating reactivation of T cells in the central nervous system. Journal of immunology (Baltimore, Md.: 1950) 192, 929-939 (2014).
15. Rothhammer, V. et al. Th17 lymphocytes traffic to the central nervous system independently of α₄ integrin expression during EAE. The Journal of experimental medicine 208, 2465-2476 (2011).
16. Sasaki, K. et al. IL-4 suppresses very late antigen-4 expression which is required for therapeutic Th1 T-cell trafficking into tumors. J Immunother 32, 793-802 (2009).
17. Ioannou, M. et al. Crucial role of granulocytic myeloid-derived suppressor cells in the regulation of central nervous system autoimmune disease. Journal of immunology (Baltimore, Md.: 1950) 188, 1136-1146 (2012).
18. Guenova, E. et al. IL-4 abrogates T (H) 17 cell-mediated inflammation by selective silencing of IL-23 in antigen-presenting cells. Proceedings of the National Academy of Sciences of the United States of America 112, 2163-2168 (2015).
19. Lotfi, N. et al. Roles of GM-CSF in the Pathogenesis of Autoimmune Diseases: An Update. Frontiers in immunology 10 (2019).
20. Luna, G. et al. Infection Risks Among Patients With Multiple Sclerosis Treated With Fingolimod, Natalizumab, Rituximab, and Injectable Therapies. JAMA Neurology (2019).
21. De Angelis, F., John, N. A. & Brownlee, W. J. Disease-modifying therapies for multiple sclerosis. BMJ 363, k4674 (2018).
22. Comi, G. et al. Efficacy of fingolimod and interferon beta-1b on cognitive, MRI, and clinical outcomes in relapsing-remitting multiple sclerosis: an 18-month, open-label, rater-blinded, randomised, multicentre study (the GOLDEN study). J Neurol 264, 2436-2449 (2017).
23. Sanford, M. & Lyseng-Williamson, K. A. Subcutaneous recombinant interferon-beta-la (Rebif (R)): a review of its use in the treatment of relapsing multiple sclerosis. Drugs 71, 1865-1891 (2011).
24. Apolloni, E. et al. Immortalized myeloid suppressor cells trigger apoptosis in antigen-activated T lymphocytes. Journal of immunology (Baltimore, Md.: 1950) 165, 6723-6730 (2000).
25. Crook, K. R. & Liu, P. Role of myeloid-derived suppressor cells in autoimmune disease. World journal of immunology 4, 26-33 (2014).
26. Komiyama, Y. et al. IL-17 Plays an Important Role in the Development of Experimental Autoimmune Encephalomyelitis. The Journal of Immunology 177, 566-573 (2006).
27. Lee, P. W. et al. IL-23R-activated STAT3/STAT4 is essential for Th1/Th17-mediated CNS autoimmunity. JCI Insight 2, e91663 (2017).
28. Liu, H. et al. Structure-based programming of lymph-node targeting in molecular vaccines. Nature 507, 519-522 (2014).
29. Yao, Z., Dai, W., Perry, J., Brechbiel, M. W. & Sung, C. Effect of albumin fusion on the biodistribution of interleukin-2. Cancer Immunol Immunother 53, 404-410 (2004).
30. Miyasaka, M. & Tanaka, T. Lymphocyte trafficking across high endothelial venules: dogmas and enigmas. Nature Reviews Immunology 4, 360-370 (2004).
31. Pyzik, M. et al. Hepatic FcRn regulates albumin homeostasis and susceptibility to liver injury. Proceedings of the National Academy of Sciences of the United States of America 114, E2862-e2871 (2017).
32. Pyzik, M. et al. The Neonatal Fc Receptor (FcRn): A Misnomer? Frontiers in immunology 10 (2019).
33. Hashem, L., Swedrowska, M. & Vllasaliu, D. Intestinal uptake and transport of albumin nanoparticles: potential for oral delivery. Nanomedicine (London, England) 13, 1255-1265 (2018).
34. Fan, Y. Y. et al. Human FcRn Tissue Expression Profile and Half-Life in PBMCs. Biomolecules 9 (2019).

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus-or step-plus-function limitations, unless such a limitation is explicitly recited in a given claim using the phrase(s) “means for” or “step for,” respectively.

1. A composition comprising a fusion protein comprising an anti-inflammatory agent operatively linked to either i) an extracellular matrix (ECM)-affinity peptide or ii) a serum protein.

2. The composition of claim 1, wherein the anti-inflammatory agent comprises a cytokine polypeptide.

3. The composition of claim 2, wherein the cytokine polypeptide comprises a polypeptide from IL-4, IL-Ira, IL-5, IL-10, IL-11, IL-23, IL-35, IL-36ra, IL-37, interferon-γ, TGF-β1, TNF receptor I, and TNF receptor II.

4. The composition of claim 1, wherein the fusion protein comprises, in order, the ECM-affinity peptide, the serum protein, and the anti-inflammatory agent.

5. The composition of claim 1, wherein the ECM-affinity peptide comprises a collagen binding domain.

6. The composition of claim 5, wherein the ECM-affinity peptide comprises a collagen binding domain from decorin or von Willebrand factor (VWF).

7. The composition of claim 1, wherein the ECM-affinity peptide comprises a collagen binding domain (CBD) from von Willebrand factor (VWF).

8. The composition of claim 1, wherein the ECM-affinity peptide comprises a peptide with an amino acid sequence that is at least 85% identical to one of SEQ ID NOS: 3, 4, 5, 47, or 52 or a peptide having an amino acid sequence that is at least 85% identical to a fragment of one of SEQ ID NOS: 3, 4, 5, 47, or 52.

9. The composition of claim 6, wherein the ECM-affinity peptide comprises a peptide with an amino acid sequence that is at least 85% identical to one of SEQ ID NOS: 3, 4, 5, 47, or 52 or a peptide having an amino acid sequence that is at least 85% identical to a fragment of one of SEQ ID NOS: 3, 4, 5, 47, or 52.

10. The composition of claim 1, wherein the operative linking is selected from covalent linking, crosslinking through a bifunctional linker and linking through a peptide bond.

11. The composition of claim 1, wherein the serum protein comprises albumin.

12. The composition of claim 6, wherein the serum protein comprises albumin.

13. The composition of claim 8, wherein the serum protein comprises albumin.

14. The composition of claim 1, wherein the ratio of ECM-affinity peptide to the anti-inflammatory agent is about 1:1 to 5:1.

15. The composition of claim 11, wherein the anti-inflammatory agent comprises a cytokine polypeptide.

16. The composition of claim 11, wherein the cytokine polypeptide comprises a polypeptide from IL-4, IL-Ira, IL-5, IL-10, IL-11, IL-23, IL-35, IL-36ra, IL-37, interferon-γ, TGF-βI, TNF receptor I, and TNF receptor II.

17. A composition comprising a fusion protein comprising an anti-inflammatory agent operatively linked to a collagen binding domain and a serum albumin protein.

18. The composition of claim 17, wherein the anti-inflammatory agent comprises a cytokine polypeptide.

19. The composition of claim 18, wherein the cytokine polypeptide comprises a polypeptide from IL-4, IL-Ira, IL-5, IL-10, IL-11, IL-23, IL-35, IL-36ra, IL-37, interferon-γ, TGF-βI, TNF receptor I, and TNF receptor II.

20. The composition of claim 17, wherein the operative linking is selected from covalent linking, crosslinking through a bifunctional linker and linking through a peptide bond.


SEQUENCE LISTING (69 sequences)

SEQ ID NO: 1 - protein, 10 aa, Bos sp.
LRELHLNNNC

SEQ ID NO: 2 - protein, 10 aa, Homo sapiens
LRELHLDNNC

SEQ ID NO: 3 - protein, 344 aa, Homo sapiens
CGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 4 - protein, 222 aa, Homo sapiens
CQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLC

SEQ ID NO: 5 - protein, 196 aa, unidentified
  REGION 1..196: note = Description of Unknown:vWF A3 sequence
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 6 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 7 - protein, 205 aa, Homo sapiens
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 8 - protein, 170 aa, unidentified
  REGION 1..170: note = Description of Unknown:PlGF-2 sequence
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 9 - protein, 30 aa, unidentified
  REGION 1..30: note = Description of Unknown:PlGF-2 sequence
RRRPKGRGKRRREKQRPTDCHLCGDAVPRR

SEQ ID NO: 10 - protein, 22 aa, unidentified
  REGION 1..22: note = Description of Unknown:PlGF-2 sequence
RRRPKGRGKRRREKQRPTDCHL

SEQ ID NO: 11 - protein, 18 aa, unidentified
  REGION 1..18: note = Description of Unknown:PlGF-2 sequence
RRPKGRGKRRREKQRPTD

SEQ ID NO: 12 - protein, 15 aa, unidentified
  REGION 1..15: note = Description of Unknown:PlGF-2 sequence
RRRPKGRGKRRREKQ

SEQ ID NO: 13 - protein, 8 aa, unidentified
  REGION 1..8: note = Description of Unknown:PlGF-2 sequence
GKRRREKQ

SEQ ID NO: 14 - protein, 8 aa, unidentified
  REGION 1..8: note = Description of Unknown:PlGF-2 sequence
RRRPKGRG

SEQ ID NO: 15 - protein, 21 aa, unidentified
  REGION 1..21: note = Description of Unknown:PlGF-2 sequence
RRKTKGKRKRSRNSQTEEPHP

SEQ ID NO: 16 - protein, 98 aa, unidentified
  REGION 1..98: note = Description of Unknown:CXCL-12gamma sequence
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 17 - protein, 30 aa, unidentified
  REGION 1..30: note = Description of Unknown:CXCL-12gamma sequence
GRREEKVGKKEKIGKKKRQKKRKAAQKRKN

SEQ ID NO: 18 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 19 - protein, 152 aa, Mus sp.
RPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 20 - protein, 129 aa, Homo sapiens [sequence omitted]

SEQ ID NO: 21 - protein, 120 aa, Mus sp.
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 22 - protein, 115 aa, Homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 23 - protein, 112 aa, Mus sp.
EIPMSTVVKETLTQLSAHRALLTSNETMRLPVPTHKNHQLCIGEIFQGLDILKNQTVRGGTVEMLFQNLSLIKKYIDRQKEKCGEERRRTRQFLDYLQEFLGVMSTEWAMEG

SEQ ID NO: 24 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 25 - protein, 101 aa, Mus sp.
LGCQALSEMIQFYLVEVMPQAEKHGPEIKEHLNSLGQKLKTLRMRLRRCHRFLPCENKSKAVEQVKSDFNKLEDQGVYKAMNEFDIFINCIEAYMMIKMKS

SEQ ID NO: 26 - protein, 178 aa, Homo sapiens
PGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 27 - protein, 179 aa, Mus sp.
MPGPPAGSPRVSSDPRADLDSAVLLTRSLLADTRQLAAQMRDKFPADGDHSLDSLPTLAMSAGTLGSLQLPGVLTRLRVDLMSYLRHVQWLRRAGGPSLKTLEPELGALQARLERLLRRLQLLMSRLALPQAAPDQPVIPLGPPASAWGSIRAAHAILGGLHLTLDWAVRGLLLLKTRL

SEQ ID NO: 28 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 29 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 30 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 209 aa, Homo sapiens
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 32 - protein, 193 aa, Mus sp.
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 33 - protein, 206 aa, Mus sp.
ALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 34 - protein, 154 aa, Homo sapiens
VLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 35 - protein, 154 aa, Mus sp.
VLSGALCFRMKDSALKVLYLHNNQLLAGGLHAEKVIKGEEISVVPNRALDASLSPVILGVQGGSQCLSCGTEKGPILKLEPVNIMELYLGAKESKSFTFYRRDMGLTSSFESAAYPGWFLCTSPEADQPVRLTQIPEDPAWDAPITDFYFQQCD

SEQ ID NO: 36 - protein, 173 aa, Homo sapiens
VHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 37 - protein, 171 aa, Mus sp.
VPSSAKVNARAPEKFSIRDQDQKVLVLSDETLIAVPNKPYTVPETFFVLASHTSSSHEGSPILLAVSKGELCLCCDKDEEQSKPSLQLKKNELMKLATQKEKVRLPFVFYRAQVGSCCTLESAAHPGWFVCTSRNSGAPVEVTDTSGEGKLMEFSFQQVSETEMSPSEVSI

SEQ ID NO: 38 - protein, 165 aa, Homo sapiens
SYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 39 - protein, 161 aa, Mus sp.
INYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 40 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 41 - protein, 112 aa, Mus sp.
ALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 42 - protein, 161 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 43 - protein, 439 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 44 - protein, 236 aa, Mus sp.
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGG

SEQ ID NO: 45 - protein, 608 aa, Homo sapiens
KWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 46 - protein, 584 aa, Mus sp.
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 47 - protein, 193 aa, unidentified
  REGION 1..193: note = Description of Unknown:vWF A3 sequence
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 48 - protein, 8 aa, synthetic construct
  REGION 1..8: note = Description of Artificial Sequence: Syntheticpeptide
GGGSGGGS

SEQ ID NO: 49 - protein, 40 aa, synthetic construct
  REGION 1..40: note = Description of Artificial Sequence: Syntheticpolypeptide
  REGION 1..40: note = MISC_FEATURE - This sequence may encompass 1-10 "Gly Gly Gly Ser"repeating units
  REGION 1..40: note = See specification as filed for detailed description ofsubstitutions and preferred embodiments
GGGSGGGSGGGSGGGSGGGSGGGSGGGSGGGSGGGSGGGS

SEQ ID NO: 50 - protein, 785 aa, synthetic construct
  REGION 1..785: note = Description of Artificial Sequence: Syntheticpolypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 51 - protein, 809 aa, synthetic construct
  REGION 1..809: note = Description of Artificial Sequence: Syntheticpolypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 52 - protein, 193 aa, unidentified
  REGION 1..193: note = Description of Unknown:vWF A3 sequence
CSQPLDVVLLLDGSSSLPESSFDKMKSFAKAFISKANIGPHLTQVSVIQYGSINTIDVPWNVVQEKAHLQSLVDLMQQEGGPSQIGDALAFAVRYVTSQIHGARPGASKAVVIIIMDTSLDPVDTAADAARSNRVAVFPVGVGDRYDEAQLRILAGPGASSNVVKLQQVEDLSTMATLGNSFFHKLCSGFSGV

SEQ ID NO: 53 - protein, 165 aa, synthetic construct
  REGION 1..165: note = Description of Artificial Sequence: Syntheticpolypeptide
RGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSHHHHHH

SEQ ID NO: 54 - protein, 758 aa, synthetic construct
  REGION 1..758: note = Description of Artificial Sequence: Syntheticpolypeptide
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGGSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSHHHHHH

SEQ ID NO: 55 - protein, 959 aa, synthetic construct
  REGION 1..959: note = Description of Artificial Sequence: Syntheticpolypeptide
CSQPLDVVLLLDGSSSLPESSFDKMKSFAKAFISKANIGPHLTQVSVIQYGSINTIDVPWNVVQEKAHLQSLVDLMQQEGGPSQIGDALAFAVRYVTSQIHGARPGASKAVVIIIMDTSLDPVDTAADAARSNRVAVFPVGVGDRYDEAQLRILAGPGASSNVVKLQQVEDLSTMATLGNSFFHKLCSGFSGVGGGSGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGGSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSHHHHHH

SEQ ID NO: 56 - protein, 127 aa, synthetic construct
  REGION 1..127: note = Description of Artificial Sequence: Syntheticpolypeptide
MHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSHHHHHH

SEQ ID NO: 57 - protein, 720 aa, synthetic construct
  REGION 1..720: note = Description of Artificial Sequence: Syntheticpolypeptide
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSGGGSMHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSHHHHHH

SEQ ID NO: 58 - protein, 202 aa, Mus sp.
QVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKG

SEQ ID NO: 59 - protein, 202 aa, Homo sapiens
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKG

SEQ ID NO: 60 - protein, 585 aa, unidentified [sequence omitted]

SEQ ID NO: 61 - protein, 795 aa, synthetic construct
  REGION 1..795: note = Description of Artificial Sequence: Syntheticpolypeptide
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 62 - protein, 403 aa, synthetic construct
  REGION 1..403: note = Description of Artificial Sequence: Syntheticpolypeptide
QVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGGGGSGGGSCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 63 - protein, 403 aa, synthetic construct
  REGION 1..403: note = Description of Artificial Sequence: Syntheticpolypeptide
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGGGGSGGGSCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 64 - protein, 995 aa, synthetic construct
  REGION 1..995: note = Description of Artificial Sequence: Syntheticpolypeptide
QVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGGGGSGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGGSCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 65 - protein, 996 aa, synthetic construct
  REGION 1..996: note = Description of Artificial Sequence: Syntheticpolypeptide
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 66 - protein, 794 aa, synthetic construct
  REGION 1..794: note = Description of Artificial Sequence: Syntheticpolypeptide
QVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGGGGSGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 67 - protein, 6 aa, synthetic construct
  REGION 1..6: note = Description of Artificial Sequence: Synthetic6xHis tag
HHHHHH

SEQ ID NO: 68 - protein, 11 aa, Homo sapiens
SYIRIADTNIT

SEQ ID NO: 69 - protein, 10 aa, synthetic construct
  REGION 1..10: note = Description of Artificial Sequence: Syntheticpeptide
  REGION 1..10: note = N-term Cy7 label
LRELHLNNNC